(19) United States
(12) Patent Application Publication
(10) Pub. No.: US 2010/0204282 A1
(43) Pub. Date: Aug. 12, 2010

(54) Reverse indoles as 5-lipoxygenase-activating protein (FLAP) inhibitors

(76) Inventors: John H. Hutchinson, La Jolla, CA (US); Nicholas Simon Stock, San Diego, CA (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/525,676

(22) PCT Filed: Feb. 4, 2008
(86) PCT No.: PCT/US08/52960

§ 371 Date: Apr. 26, 2010

Related U.S. Application Data
(60) Provisional application No. 60/888,157, filed on Feb. 5, 2007.

(51) Int. Cl. A61K 31/4439; C07D 401/12; A61P 11/00; A61P 11/06

(52) U.S. Cl. 514/357; 546/344

(57) ABSTRACT

Described herein are compounds and pharmaceutical compositions containing such compounds, which modulate the activity of 5-lipoxygenase-activating protein (FLAP). Also described herein are methods of using such FLAP modulators, alone and in combination with other compounds, for treating respiratory, cardiovascular, and other leukotriene-dependent or leukotriene mediated conditions or diseases.

### CROSS-REFERENCE

This application claims the benefit of U.S. Provisional Application No. 60/888,157, filed Feb. 5, 2007, which application is incorporated herein by reference in its entirety.

### FIELD OF THE INVENTION

Described herein are compounds, methods of making such compounds, pharmaceutical compositions and medicaments that include such compounds, and methods of using such compounds to treat or prevent diseases or conditions associated with 5-lipoxygenase-activating protein (FLAP) activity.

### BACKGROUND OF THE INVENTION

The protein 5-lipoxygenase-activating protein (FLAP) is associated with the pathway of leukotriene synthesis. In particular, 5-lipoxygenase-activating protein (FLAP) is responsible for binding arachidonic acid and transferring it to 5-lipoxygenase. 5-Lipoxygenase can then catalyze the two-step oxygenation and dehydration of arachidonic acid, converting it into the intermediate compound 5-HPETE (5-hydroperoxyeicosatetraenoic acid), and in the presence of FLAP convert the 5-HPETE to Leukotriene A₄ (LTA₄). Abramovitz, M. et al., Eur. J. Biochem. 215:105-111 (1993) is hereby incorporated by reference for such disclosure.

Leukotrienes are biological compounds formed from arachidonic acid in the leukotriene synthesis pathway. Leukotrienes are synthesized primarily by eosinophils, neutrophils, mast cells, basophils, dendritic cells, macrophages and monocytes. Leukotrienes have been implicated in biological actions including, by way of example only, smooth muscle contraction, leukocyte activation, cytokine secretion, mucous secretion, and vascular function. Samuelsson et al, Science, 220, 568-575, (1983) and Cooper, The Cell, A Molecular Approach, 2nd Ed. Sinauer Associates, Inc., Sunderland (MA), (2000) are hereby incorporated by reference for such disclosure.

### SUMMARY OF THE INVENTION

Certain embodiments presented herein provide for methods, compounds, pharmaceutical compositions, and medicaments for (a) diagnosing, preventing, and/or treating allergic and non-allergic inflammation, (b) controlling signs and symptoms that are associated with inflammation, and/or (c) controlling proliferative or metabolic disorders. In certain embodiments, these disorders arise from, by way of non-limiting example, genetic, iatrogeic, immunological, infectious, metabolic, oncologic, toxic, and/or traumatic etiology. In one aspect, the methods, compounds, pharmaceutical compositions, and medicaments described herein include 5-lipoxygenase-activating protein (FLAP) inhibitors described herein.

In one aspect provided herein are compounds of Formula (A), pharmaceutically acceptable salts, pharmaceutically acceptable N-oxides, pharmaceutically active metabolites, pharmaceutically acceptable prodrugs, and pharmaceutically acceptable solvates thereof, which antagonize or inhibit FLAP. In certain embodiments compounds of Formula (A) are used to treat patients suffering from leukotriene-dependent conditions or diseases, including, but not limited to, asthma, chronic obstructive pulmonary disease, pulmonary hypertension, interstitial lung fibrosis, rhinitis, arthritis, allergy, psoriasis, inflammatory bowel disease, adult respiratory distress syndrome, myocardial infarction, aneurysm, stroke, cancer, endotoxic shock, proliferative disorders and inflammatory conditions.

Formula (A) is as follows:

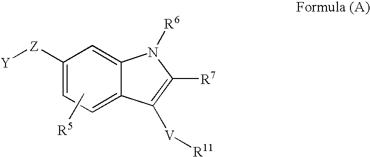

wherein:
Z is selected from —[C(R¹)₂]ₘ—[C(R²)₂]ₙ, —[C(R²)₂]ₙ—, —[C(R¹)₂]ₘ—O—, —O—[C(R¹)₂]ₘ—[C(R²)₂]ₙ—, —[C(R²)₂]ₙ—O—[C(R¹)₂]ₙ—, and —[C(R¹)₂]ₙ—O—[C(R²)₂]ₙ—;
   each R¹ is independently H, —CF₃, or an optionally substituted C₁-C₄alkyl; or
   two R² on the same carbon taken together with the carbon to which they are attached form a carbonyl (C═O);
   each R² is independently H, —OH, —OMe, —CF₃, or an optionally substituted C₁-C₄alkyl; or
   each R² can be (L^{s}R^{s}); or
   two R² on the same carbon taken together with the carbon to which they are attached form a carbonyl (C═O);
   m is 0, 1 or 2;
   each n is independently 0, 1, 2, or 3;
Y is H, -(substituted or unsubstituted C₁-C₆alkyl), -(substituted or unsubstituted C₃-C₈cycloalkyl), -(substituted or unsubstituted aryl), -(substituted or unsubstituted heteroaryl), or -(substituted or unsubstituted heterocycloalkyl);
where if Y or Z are substituted, then each substitutent on Y or Z is independently (L^{s}R^{s}),
   each L^{s} is independently selected from among a bond, —O—, —C(═O)—, —S—, —S(═O)—, —S(═O)₂—, —NHC(═O)—, —C(═O)NH—, S(═O)₂NH—, —NHS(═O)₂, —OC(═O)NH—, —NHC(═O)O—, —OC(═O)O—, —NHC(═O)NH—, —C(═O)O—, —OC(═O)—, (substituted or unsubstituted C₁-C₆alkyl), (C₂-C₆alkenyl), (C₁-C₆-fluoroalkyl), (substituted or unsubstituted heteroaryl), (substituted or unsubstituted aryl), and (substituted or unsubstituted heterocycloalkyl);
   each R^{s} is independently selected from among H, halogen, —N(R⁹)₂, —CN, —NO₂, —N₃, —S(═O)₂NH₂, (substituted or unsubstituted C₁-C₆alkyl), (substituted or unsubstituted C₃-C₈cycloalkyl), (C₁-C₆fluoroalkyl), (substituted or unsubstituted aryl), (substituted or unsubstituted heteroaryl), and (substituted or unsubstituted C₁-C₆heteroalkyl);
R⁵ is H, halogen, substituted or unsubstituted C₁-C₆alkyl, or substituted or unsubstituted —O—(C₁-C₆alkyl);
R⁶ is H, -L²-(substituted or unsubstituted C₁-C₆alkyl), -L²-(substituted or unsubstituted C₃-C₈cycloalkyl), -L²-(substituted or unsubstituted C₂-C₆alkenyl), -L²-(substituted or unsubstituted C₅-C₈cycloalkenyl), -L²-(substituted or unsubstituted heterocycloalkyl), -L²-(substituted or unsubstituted heteroaryl), or -L²-(substituted or unsubstituted aryl); and
   L² is a bond, —S(═O)₂—, —C(═O)—, or -(substituted or unsubstituted C₁-C₆alkyl)-;
R⁷ is L³-X-L⁴-G¹, wherein,
   L³ is a substituted or unsubstituted C₁-C₆alkyl;
   X is a bond, —O—, —C(═O)—, —CR⁹(OR⁹)—, —S—, —S(═O)—, —S(═O)₂—, —NR⁹—, —NR⁹C(═O)—, —C(═O)NR⁹—, aryl, heteroaryl or —NR⁹C(═O)NR⁹—;
   L⁴ is a bond, or a substituted or unsubstituted C₁-C₆alkyl;
   G¹ is H, tetrazolyl, —NHS(═O)₂R⁸, S(═O)₂N(R⁹)₂, —OR⁹, —C(═O)CF₃, —C(═O)NHS(═O)₂R⁸, —S(═O)₂NHC(═O)R⁹, —CN, —N(R⁹)₂, —N(R⁹)C(═O)R⁸, —C(═NR¹⁰)N(R⁹)₂, —NR⁹C(═NR¹⁰)N(R⁸)₂, —NR⁹C(═CR¹⁰)N(R⁹)₂, —C(═O)NR⁹C(═NR¹⁰)N(R⁹)₂, —C(═O)NR⁹C(═CR¹⁰)N(R⁹)₂, —CO₂R⁹, —C(═O)R⁹, —CON(R⁹)₂, —SR⁸, —S(═O)R⁸, —S(═O)₂R⁸, -L⁵-(substituted or unsubstituted C₁-C₆alkyl), -L⁵-(substituted or unsubstituted C₂-C₆alkenyl), -L⁵-(substituted or unsubstituted heteroaryl), or -L⁵-(substituted or unsubstituted aryl);
      L⁵ is —OC(═O)O—, —NHC(═O)NH—, —NHC(═O)O, —OC(═O)NH—, —NHC(═O), —C(═O)NH, —C(═O)O—, or —OC(═O)—;
   or G′ is W-G², where
      W is a (substituted or unsubstituted aryl), (substituted or unsubstituted heterocycloalkyl), or (substituted or unsubstituted heteroaryl); and
      G² is H, tetrazolyl, —NHS(═O)₂R⁸, S(═O)₂N(R⁹)₂, OH, —OR⁸, —C(═O)CF₃, —C(═O)NHS(═O)₂R⁸, —S(═O)₂NHC(O)R⁹, CN, N(R⁹)₂, —N(R⁹)C(═O)R⁸, —C(═NR¹⁰)N(R⁹)₂, —NR⁹C(═NR¹⁰)N(R⁹)₂, —NR⁹C(═CR¹⁰)N(R⁹)₂, —C(═O)NR⁹C(═NR¹⁰)N(R⁹)₂, —C(═O)NR⁹C(═CR¹⁰)N(R⁹)₂, —CO₂R⁹, —C(═O)R⁹, —CON(R⁹)₂, —SR, —S(═O)R⁸, or —S(═O)₂R⁸;
      each R⁸ is independently selected from among (substituted or unsubstituted C₁-C₄alkyl), (substituted or unsubstituted C₃-C₈cycloalkyl), (substituted or unsubstituted phenyl), (substituted or unsubstituted heteroaryl), and (substituted or unsubstituted benzyl);
      each R⁹ is independently selected from among H, (substituted or unsubstituted C₁-C₄alkyl), (substituted or unsubstituted C₃-C₈cycloalkyl), (substituted or unsubstituted phenyl), (substituted or unsubstituted heteroaryl), and (substituted or unsubstituted benzyl); or
      two R⁹ groups can together form a 5-, 6-, 7-, or 8-membered heterocyclic ring; or
      R⁸ and R⁹ can together form a 5-, 6-, 7-, or 8-membered heterocyclic ring; and
      each R¹⁰ is independently selected from H, —S(═O)₂R⁸, —S(═O)₂NH₂, —C(O)R⁸, —CN, —NO₂, heteroaryl, or heteroalkyl;
R¹¹ is L⁷-L¹⁰-G³, wherein
   L⁷ is a bond, or (substituted or unsubstituted C₁-C₆alkyl);
   L¹⁰ is a bond, (substituted or unsubstituted C₁-C₆alkyl), (substituted or unsubstituted C₃-C₈cycloalkyl), (substituted or unsubstituted heteroaryl), (substituted or unsubstituted aryl), or (substituted or unsubstituted heterocycloalkyl);
   G³ is —OR⁹, —C(═O)R⁹, —C(═O)OR⁹, —SR, —S(═O)R⁸, —S(9)₂R⁸, —N(R⁹)₂, tetrazolyl, —NHS(═O)₂R⁸, —S(═O)₂N(R⁹)₂, —C(═O)NHS(═O)₂R⁸, —S(═O)₂NHC(═O)R⁸, —C(═O)N(R⁹)₂, —NR⁹C(═O)R⁸, —C(R⁹)₂C(═O)N(R⁹)₂, —C(═NR¹⁰)N(R⁹)₂, —NR⁹C(═NR¹⁰)N(R⁹)₂, —NR⁹C(═CR¹⁰)N(R⁹)₂, -L⁸-(substituted or unsubstituted C₁-C₆alkyl), -L⁸-(substituted or unsubstituted C₂-C₆alkenyl), -L⁸-(substituted or unsubstituted heteroaryl), or -L⁸-(substituted or unsubstituted aryl);
      L⁸ is —O—, —C(═O)—, —S—, —S(═O)—, —S(═O)₂—, —NH—, —NHC(═O)O—, —NHC(═O)NH—, —OC(═O)O—, —OC(═O)NH—, —NHC(═O)—, —C(═O)NH—, —C(═O)O—, or —OC(═O)—;
   or G³ is W⁴-G⁴, wherein
      W⁴ is (substituted or unsubstituted C₁-C₆alkyl), (substituted or unsubstituted heterocycloalkyl), (substituted or unsubstituted aryl) or a (substituted or unsubstituted heteroaryl); and
      G⁴ is H, halogen, —CN, —NO₂, —N₃, —CF₃, —OCF₃, C₁-C₆alkyl, C₃-C₈cycloalkyl, C₁-C₆fluoroalkyl, tetrazolyl, —NHS(O)R⁸, —S(═O)₂N(R⁹)₂, —OH, —OR⁸, —C(═O)CF₃, —C(O)NHS(═O)₂R⁸, —S(═O)₂NHC(═O)R⁸, —CN, —N(R⁹)₂, —N(R⁹)C(O)R⁸, —C(═NR¹⁰)N(R⁹)₂, —NR⁹C(═NR¹⁰)N(R⁹)₂, —NR⁹C(═CR¹⁰)N(R⁹)₂, —C(O)NR⁹C(═NR¹⁰)N(R⁹)₂, —C(O)NR⁹C(═CR¹⁰)N(R⁹)₂, —CO₂R⁹, —C(═O)R⁹, —CON(R⁹)₂, —SR⁸, —S(═O)R⁸, —S(═O)₂R⁸, -L⁹-(substituted or unsubstituted C₁-C₆alkyl), -L⁹-(substituted or unsubstituted C₂-C₆alkenyl), -L⁹-(substituted or unsubstituted C₁-C₆heteroalkyl), -L⁹-(substituted or unsubstituted heteroaryl), -L⁹-(substituted or unsubstituted heterocycloalkyl), or -L⁹-(substituted or unsubstituted aryl);
         L⁹ is a bond, —O—, C(═O), —S—, —S(═O)—, —S(═O)₂—, —NH—, —NHC(═O)O—, —NHC(═O)NH—, —OC(═O)O—, —OC(═O)NH—, —NHC(═O)—, —C(═O)NH—, —C(═O)O—, or —OC(═O)—;
   provided that R¹¹ comprises at least one (unsubstituted or substituted aromatic moiety) and at least one (unsubstituted or substituted heterocyclic moiety), wherein the (unsubstituted or substituted heterocyclic moiety) is a (unsubstituted or substituted heterocycloalkyl moiety) or a (unsubstituted or substituted heteroaryl moiety), and R¹¹ is not a thienyl-phenyl group;
V is a bond, —C(═O)—, —C(OH)R¹³—, —(CR¹²R¹³)—, —NH—, —C(═O)NH—, —NHC(═O)—, —S—, —S(═O)—, or —S(═O)₂—;
R¹² is H, halogen, (substituted or unsubstituted C₁-C₆alkyl), (substituted or unsubstituted C₃-C₈cycloalkyl);
R¹³ is H, (substituted or unsubstituted C₁-C₆alkyl); or
R¹² and R¹³ taken together with the carbon to which they are attached may join to form a C₃-C₈cycloalkyl; or
active metabolites, pharmaceutically acceptable solvates, pharmaceutically acceptable salts, pharmaceutically acceptable N-oxides, or pharmaceutically acceptable prodrugs thereof.

For any and all of the embodiments, substituents can be selected from among from a subset of the listed alternatives. For example, in some embodiments, Z is selected from among —[C(R¹)₂]ₘ—[C(R²)₂]ₙ—, —[C(R²)₂]ₙ—[C(R¹)₂]ₘ—O—, and —O—[C(R¹)₂]m—[C(R²)₂]ₙ—; and Y is H, -(substituted or unsubstituted C₁-C₆alkyl), (substituted or unsubstituted aryl), -(substituted or unsubstituted heteroaryl), or -(substituted or unsubstituted heterocycloalkyl). In further or alternative embodiments, Z is selected from among —C(R¹)₂—[C(R²)₂]ₙ—, —[C(R²)₂]ₙ—[C(R¹)₂]ₘ—O—, and —O—[C(R²)₂]ₙ—[C(R²)₂]ₙ—. In further or alternative embodiments, each R¹ is independently H, —CF₃, or —CH₃. In further or alternative embodiments, Z is selected from among —CH₂—[C(R²)₂]ₙ—, —CH(Me)—[C(R²)₂]ₙ—, —[C(R²)₂]ₙ—CH₂—O—, —[C(R²)₂]ₙ—CH(Me)—O—, —O—CH₂—[C(R²)₂]ₙ—, and —O—CH(Me)—[C(R²)₂]ₙ—. In further or alternative embodiments, Z is selected from among —CH₂—, —CH(Me)—, —CH₂—O—, —CH(Me)—O—, —O—CH₂—, and —O—CH(Me)—. In further or alternative embodiments, Z is —[C(R₂)₂]ₙC(R₁)₂O—.

In further or alternative embodiments, each R² is independently H, —CF₃, or an optionally substituted C₁-C₄alkyl.

In further or alternative embodiments, Y is H, -(substituted or unsubstituted C₁-C₆alkyl), -(substituted or unsubstituted aryl), -(substituted or unsubstituted heteroaryl), or -(substituted or unsubstituted heterocycloalkyl). In further or alternative embodiments, Y is H, -(substituted or unsubstituted C₁-C₆alkyl), -(substituted or unsubstituted aryl), -(substituted or unsubstituted heteroaryl containing 0-1 S atoms, 0-1 O atoms, and 0-3 N atoms), or -(substituted or unsubstituted heterocycloalkyl containing 0-2 N atoms).

In further or alternative embodiments, Y is H, -(substituted or unsubstituted C₁-C₆alkyl), or -(substituted or unsubstituted aryl).

In further or alternative embodiments, Y is a -(substituted or unsubstituted heteroaryl), or -(substituted or unsubstituted heterocycloalkyl).

In further or alternative embodiments, Y is a -(substituted or unsubstituted heteroaryl). In further or alternative embodiments, Y is a -(substituted or unsubstituted heteroaryl containing 0-1 S atoms, 0-1 O atoms, and 0-3 N atoms). In further or alternative embodiments, Y is a substituted or unsubstituted group selected from among pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, naphthyridinyl, imidazo[1,2-a]pyridinyl, thiophenopyridinyl, and furopyridinyl.

In further or alternative embodiments, Y is a substituted or unsubstituted group selected from among pyridinyl, pyrimidinyl, pyrazinyl, quinolinyl, isoquinolinyl, indolyl, benzimidazolyl, benzofuranyl, cinnolinyl, indazolyl, indolizinyl, phthalazinyl, pyridazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, naphthyridinyl, imidazo[1,2-a]pyridinyl, thiophenopyridinyl, and furopyridinyl. In further or alternative embodiments, Y is a substituted or unsubstituted group selected from among pyridinyl, pyrimidinyl, pyrazinyl, quinolinyl, isoquinolinyl, pyridazinyl, quinazolinyl, quinoxalinyl. In further or alternative embodiments, Y is a substituted or unsubstituted group selected from among pyridinyl, and quinolinyl.

In further or alternative embodiments, Y is a -(substituted or unsubstituted heterocycloalkyl). In some embodiments, Y is a substituted or unsubstituted group selected from among quinolizinyl, dioxinyl, piperidinyl, morpholinyl, thiazinyl, tetrahydropyridinyl, piperazinyl, oxazinanonyl, dihydropyrrolyl, dihydroimidazolyl, tetrahydrofuranyl, dihydrooxazolyl, oxiranyl, pyrrolidinyl, pyrazolidinyl, dihydrothienyl, imidazolidinonyl, pyrrolidinonyl, dihydrofuranonyl, dioxolanonyl, thiazolidinyl, piperidinonyl, indolinyl, indanyl, tetrahydronaphthalenyl, tetrahydroquinolinyl, tetrahydrothienyl, and thiazepanyl.

In further or alternative embodiments, Y is selected from among:

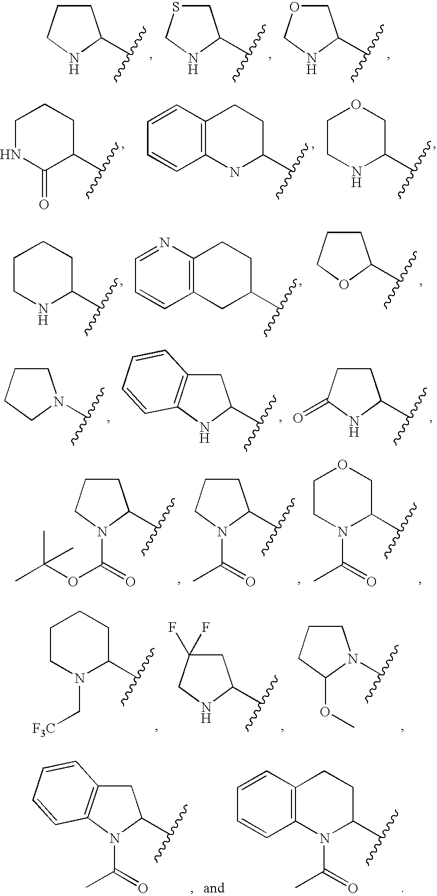

In further or alternative embodiments, R⁵ is H, halogen, —CH₃, or —OCH₃. In further or alternative embodiments, R⁵ is H.

In further or alternative embodiments, R⁶ is H, -L²-(substituted or unsubstituted C₁-C₆alkyl), -L²-(substituted or unsubstituted C₃-C₈cycloalkyl), -L²-(substituted or unsubstituted heterocycloalkyl), -L²-(substituted or unsubstituted heteroaryl), or -L²-(substituted or unsubstituted aryl); and L² is a bond, —S(═O)₂—, —C(═O)—, or -(substituted or unsubstituted C₁-C₆alkyl)-. In further or alternative embodiments, L² is a bond, —C(═O)—, or -(substituted or unsubstituted C₁-C₆alkyl)-.

In further or alternative embodiments, R⁶ is H, -L²-(substituted or unsubstituted C₁-C₆allyl), -L²-(substituted or unsubstituted C₃-C₈cycloalkyl), or -L²-(substituted or unsubstituted aryl).

In further or alternative embodiments, V is a bond, —C(═O)—, —C(OH)R¹³—, —CR¹²R¹³—, —NH—, —C(═O)NH, —NHC(═O)—, —S—, —S(═O)—, or —S(═O)₂—; R¹² is H, halogen, or (substituted or unsubstituted C₁-C₆alkyl); R¹³ is H, (substituted or unsubstituted C₁-C₆alkyl). In further or alternative embodiments, V is a bond, —C(═O)—, —C(OH)H—, —CR¹²H—, —NH—, —C(═O)NH, —NHC(═O)—, —S—, —S(═O)—, or —S(═O)₂—. In further or alternative embodiments, V is a bond, —C(═O)—, —C(OH)H—, —CR¹²H—, —S—, —S(O)—, or —S(═O)₂—. In further or alternative embodiments, V is —C(═O)—, —C(OH)H—, —CR¹²H—, —S—, —S(═O)—, or —S(═O)₂—.

In further or alternative embodiments, X is a bond, —O—, —C(═O)—, —CR⁹(OR⁹)—, —NR⁹—, —NR⁹C(═O)—, —C(═O)NR⁹—, aryl, heteroaryl or —NR⁹C(═O)NR⁹—.

In further or alternative embodiments, X is a bond, —O—, —C(═O)—, —CR⁹(OR⁹)—, —NR⁹—, —NR⁹C(═O)—, —C(═O)NR⁹—, or —NR⁹C(═O)NR⁹—.

In further or alternative embodiments, X is a bond, —O—, —C(═O)—, —CH(OH)—, —NH—, —NHC(═O)—, —C(═O)NH—, or —NHC(═O)NH—.

In further or alternative embodiments, X is a bond.

In further or alternative embodiments, G¹ is H, tetrazolyl, —OR⁹, —C(═O)NHS(═O)₂R⁸, —S(═O)₂NHC(═O)R⁸, —CN, —N(R⁹)₂, —N(R⁹)C(═O)R⁸, —NR⁹C(═CR¹⁰)N(R⁹)₂, —CO₂R⁹, —C(═O)R⁹, —CON(R⁹)₂, —SR⁸, —S(═O)R⁸, —S(═O)₂R⁸, -L⁵-(substituted or unsubstituted C₁-C₆alkyl), -L⁵-(substituted or unsubstituted heteroaryl), or -L⁵-(substituted or unsubstituted aryl); and L⁵ is —NHC(═O)O, —NHC(═O), —C(O)NH, —C(═O)O—, or —OC(═O)—.

In further or alternative embodiments, G¹ is H, tetrazolyl, —OR⁹, —CN, —N(R⁹)C(═O)R⁸, —CO₂R⁹, —C(═O)R⁹, —CON(R⁹)₂, —S(═O)R⁸, —S(═O)₂R⁸, -L⁵-(substituted or unsubstituted C₁-C₆alkyl), -L⁵-(substituted or unsubstituted heteroaryl), or -L⁵-(substituted or unsubstituted aryl).

In further or alternative embodiments, G¹ is H, tetrazolyl, —OR⁹, —CN, —N(R⁹)C(═O)R⁹, —CO₂R⁹, —C(═O)R⁹, —CON(R⁹)₂, —SR⁸, —S(═O)R⁸, —S(═O)₂R⁸, -L⁵-(substituted or unsubstituted C₁-C₆alkyl), -L⁵-(substituted or unsubstituted heteroaryl).

In further or alternative embodiments, G¹ is H, tetrazolyl, —OR⁹, —CN, —CO₂R⁹, —CON(R⁹)₂, -L⁵-(substituted or unsubstituted C₁-C₆alkyl), -L⁵-(substituted or unsubstituted heteroaryl).

In further or alternative embodiments, G¹ is H, tetrazolyl, —OR⁹, —CO₂R⁹, —CON(R⁹)₂, -L⁵-(substituted or unsubstituted C₁-C₆alkyl), -L⁵-(substituted or unsubstituted heteroaryl).

In further or alternative embodiments, G¹ is W-G².

In further or alternative embodiments, W is a (substituted or unsubstituted heterocycloalkyl), or a (substituted or unsubstituted heteroaryl); G² is H, tetrazolyl, —NHS(═O)₂R⁸, S(═O)₂N(R⁹)₂, OH, —OR⁸, —C(O)CF₃, CN, N(R⁹)₂, —N(R⁹)C(═O)R⁸, —C(NR¹⁰)N(R⁹)₂, —CO₂R⁹, —C(═O)R⁹, —CON(R⁹)₂, —SR⁸, —S(═O)R⁸, or —S(═O)₂R⁸.

In further or alternative embodiments, G² is H, tetrazolyl, —OH, —OR⁸, —C(═O)CF₃, CN, N(R⁹)₂, —N(R⁹)C(═O)R⁸, —CO₂R⁹, —C(═O)R⁹, —CON(R⁹)₂.

In further or alternative embodiments, each R⁸ is a (substituted or unsubstituted C₁-C₄alkyl); and each R⁹ is independently selected from among H, and (substituted or unsubstituted C₁-C₄alkyl).

In further or alternative embodiments, L⁷ is a bond; and L¹⁰ is a (substituted or unsubstituted heteroaryl), (substituted or unsubstituted aryl), or (substituted or unsubstituted heterocycloalkyl).

In further or alternative embodiments, G³ is tetrazolyl, -L⁸-(substituted or unsubstituted heteroaryl), or -L⁸-(substituted or unsubstituted aryl); and L⁸ is —O—, —C(═O)—, —C(═O)NH—, —C(═O)O—, or —OC(═O)—.

In further or alternative embodiments, G³ is W⁴-G⁴.

In further or alternative embodiments, W⁴ is (substituted or unsubstituted heterocycloalkyl), (substituted or unsubstituted aryl) or a (substituted or unsubstituted heteroaryl); and G⁴ is H, halogen, —CN, —CF₃, —OCF₃, C₁-C₆alkyl, C₃-C₈cycloalkyl, C₁-C₆fluoroalkyl, tetrazolyl, —OH, —OR⁸, —C(═O)CF₃, —CN, —CO₂R⁹, —C(═O)R⁹, —CON(R⁹)₂, -L⁹-(substituted or unsubstituted C₁-C₆alkyl), -L⁹-(substituted or unsubstituted C₁-C₆heteroalkyl), -L⁹-(substituted or unsubstituted heteroaryl), -L⁹-(substituted or unsubstituted heterocycloalkyl), or -L⁹-(substituted or unsubstituted aryl); and L⁹ is a bond, —O—, C(═O), —S—, —S(═O)—, —S(═O)₂—, —NH—, —NHC(═O)O—, —OC(═O)O—, —OC(═O)NH—, —NHC(═O)—, —C(═O)NH—, —C(═O)O—, or —OC(═O)—.

In further or alternative embodiments, L¹⁰ is (substituted or unsubstituted aryl); and G³ is W⁴-G⁴, where W⁴ is (substituted or unsubstituted heterocycloalkyl), or a (substituted or unsubstituted heteroaryl).

In further or alternative embodiments, provided herein is a compound having the structure of Formula (B):

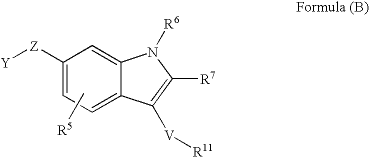

wherein:
Z is selected from —O—[C(R¹)₂]ₘ—[C(R²)₂]ₙ— and —[C(R²)₂]ₙ—[C(R¹)₂]ₘ—O—;
   each R¹ is independently H, —CF₃, or an optionally substituted C₁-C₄alkyl; or
two R¹ on the same carbon taken together with the carbon to which they are attached form a carbonyl (C═O);
   each R² is independently H, —OH, —OMe, —CF₃, or an optionally substituted C₁-C₄alkyl; or
two R² on the same carbon taken together with the carbon to which they are attached form a carbonyl (C═O);
   m is 0, 1 or 2;
   n is 0, 1, 2, or 3;
Y is -(substituted or unsubstituted aryl), -(substituted or unsubstituted heteroaryl); or (substituted or unsubstituted heterocycloalkyl);
wherein if Y is substituted, then each substitutent of Y is independently (L^{s}R^{s}),
   each L^{s} is independently selected from a bond and —C(═O)—;
   each R^{s} is independently selected from halogen and C₁-C₆alkyl;
R⁵ is H, halogen, substituted or unsubstituted C₁-C₆alkyl, or substituted or unsubstituted —O—(C₁-C₆alkyl);
R⁶ is selected from C₁-C₆alkyl, C₃-C₈cycloalkyl, C₁-C₆allyl-C₃-C₈cycloalkyl, aryl, and C₁-C₆alkyl-aryl; and
R⁷ is -L³-G¹;
   L³ is C₁-C₆alkyl;
   G¹ is selected from H, tetrazolyl, —C(═NH)N(R⁹)₂, —NR⁹C(═NH)N(R⁹)₂, —NR⁹C(═CH₂)N(R⁹)₂, —C(═O)NR⁹C(═NH)N(R⁹)₂, —C(═O)NR⁹C(═CH₂)N(R⁹)₂, —CO₂R⁹, and —CON(R⁹)₂;
      each R⁹ is independently selected from H and C₁-C₄alkyl;
R¹¹ is -L¹⁰-W⁴-G⁴, wherein
   L¹⁰ is a substituted or unsubstituted aryl;
   W⁴ is a -(substituted or unsubstituted heteroaryl) or a -(substituted or unsubstituted heterocycloalkyl); and
   G⁴ is H, halogen, C₁-C₆alkyl, C₁-C₆alkoxy, or —CF₃;
V is a bond, —(CR¹²R¹³)—, —S—, —S(═O)—, or —S(═O)₂—;
R¹² is H, halogen, or C₁-C₆alkyl;
R¹³ is H, or C₁-C₆alkyl; or
R¹² and R¹³ taken together with the carbon to which they are attached may join to form a C₃-C₈cycloalkyl; or
active metabolites, pharmaceutically acceptable solvates, pharmaceutically acceptable salts, pharmaceutically acceptable N-oxides, or pharmaceutically acceptable prodrugs thereof.

In further or alternative embodiments, provided herein is a compound having the structure of Formula (C):

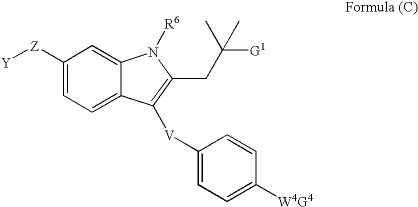

wherein:
Z is selected from —O—[C(R¹)₂]ₘ—[C(R²)₂]ₙ— and —[C(R²)₂]ₙ—[C(R¹)₂]ₘ—O—;
   each R¹ is independently H, —CF₃, or an optionally substituted C₁-C₄alkyl; or
   two R¹ on the same carbon taken together with the carbon to which they are attached form a carbonyl (C═O);
   each R² is independently H, —OH, —OMe, —CF₃, or an optionally substituted C₁-C₄alkyl; or
   two R² on the same carbon taken together with the carbon to which they are attached form a carbonyl (C═O);
   m is 0, 1 or 2;
   n is 0, 1, 2, or 3;
Y is -(substituted or unsubstituted aryl), -(substituted or unsubstituted heteroaryl); or (substituted or unsubstituted heterocycloalkyl);
wherein if Y is substituted, then each substitutent of Y is independently (L^{s}R^{s}),
   each L^{s} is independently selected from a bond and —C(═O)—;
   each R^{s} is independently selected from halogen and C₁-C₆alkyl;
R⁶ is selected from C₁-C₆alkyl, C₃-C₈cycloalkyl, C₁-C₆alkyl-C₃-C₈cycloalkyl, aryl, and C₁-C₆alkyl-aryl; and
G¹ is selected from H, tetrazolyl, —C(═NH)N(R⁹)₂, —NR⁹C(═NH)N(R⁹)₂, —NR⁹C(═CH₂)N(R⁹)₂, —C(═O)NR⁹C(═NH)N(R⁹)₂, —C(═O)NR⁹C(═CH₂)N(R⁹)₂, —CO₂R⁹, and —CON(R⁹)₂;
   each R⁹ is independently selected from H and C₁-C₄alkyl;
W⁴ is a -(substituted or unsubstituted heteroaryl) or a -(substituted or unsubstituted heterocycloalkyl); and
G⁴ is H, halogen, C₁-C₆alkyl, C₁-C₆alkoxy, or —CF₃;
V is a bond, —(CR¹²R¹³)—, —S—, —S(═O)—, or —S(═O)₂—;
   R¹² is H, halogen, or C₁-C₆alkyl;
   R¹³ is H, or C₁-C₆alkyl; or
   R¹² and R¹³ taken together with the carbon to which they are attached may join to form a C₃-C₈cycloalkyl; or
active metabolites, pharmaceutically acceptable solvates, pharmaceutically acceptable salts, pharmaceutically acceptable N-oxides, or pharmaceutically acceptable prodrugs thereof.

In further or alternative embodiments, Z is selected from, by way of non-limiting example, —O(CH₂)ₘ(CH₂)ₙ— and —(CH₂)ₙ(CH₂)ₘO—. In specific embodiments, Z is selected from, by way of non-limiting example, —OCH₂— and —CH₂O—. In further or alternative embodiments, G¹ is selected from tetrazolyl and —CO₂R⁹. In further or alternative embodiments, V is selected from, by way of non-limiting example, —CH₂—, —S—, —S(═O)— and —S(═O)₂—.

Any combination of the groups described above for the various variables is contemplated herein. In further or alternative embodiments, substituents and substitution patterns on the compounds provided herein are selected so as to provide chemically stable compounds. Such compounds are prepared in any suitable manner.

In further or alternative aspects, provided herein is a pharmaceutical composition comprising an effective amount of a compound provided herein, and a pharmaceutically acceptable excipient.

In further or alternative aspects, provided herein is a method for treating inflammation in a mammal that includes administering a therapeutically effective amount of a compound provided herein to the mammal in need. In one embodiment, the mammal is a human.

In further or alternative aspects, provided herein is a method for treating asthma in a mammal that includes administering a therapeutically effective amount of a compound provided herein to the mammal in need. In a further or alternative embodiment, provided herein is a method for treating asthma in a mammal that includes administering a therapeutically effective amount of a compound provided herein to a mammal in need thereof. In a specific embodiment, the compound is a compound of Formula (A), wherein Z is [C(R₂)₂]ₙC(R₁)₂O.

In further or alternative embodiments, provided herein are compounds presented in FIGS. 6, 7, and 8 and Table 1, or pharmaceutically acceptable salts, pharmaceutically acceptable N-oxides, pharmaceutically active metabolites, pharmaceutically acceptable prodrugs, and pharmaceutically acceptable solvates thereof. In specific embodiments, compounds described herein antagonize or inhibit FLAP. In further or alternative embodiments, compounds described herein are used to treat patients suffering from leukotriene-dependent conditions or diseases, including, but not limited to, asthma, chronic obstructive pulmonary disease, pulmonary hypertension, interstitial lung fibrosis, rhinitis, arthritis, allergy, psoriasis, inflammatory bowel disease, adult respiratory distress syndrome, myocardial infarction, aneurysm, stroke, cancer, endotoxic shock, proliferative disorders and inflammatory conditions.

In further or alternative embodiments, compounds provided herein are inhibitors of leukotriene biosynthesis. In some embodiments, the compounds described herein, including those of Formula (A), (B) or (C), are inhibitors of 5-lipoxygenase-activating protein (FLAP), while in specific embodiments, such inhibitors are selective for FLAP. In further or alternative embodiments, compounds described herein have an IC₅₀ below 50 microM in a FLAP binding assay.

In further or alternative embodiments, the compounds of Formula (A), (B) or (C) are included into pharmaceutical compositions or medicaments used for treating a leukotriene-dependent or leukotriene mediated condition or disease in a patient.

In further or alternative aspects, provided herein are pharmaceutical compositions that include a compound, pharmaceutically acceptable salt, pharmaceutically acceptable N-oxide, pharmaceutically active metabolite, pharmaceutically acceptable prodrug, or pharmaceutically acceptable solvate of any compound described herein. In further or alternative aspects, provided herein are compositions further including a pharmaceutically acceptable diluent, excipient or binder. In further or alternative aspects, provided are compositions further including a second pharmaceutically active ingredient.

In further or alternative embodiments, provided herein is a pharmaceutical composition containing: i) a physiologically acceptable carrier, diluent, and/or excipient; and ii) one or more compounds provided herein.

In further or alternative aspect inflammatory conditions treated by administering a compound described herein include, but are not limited to, asthma, chronic obstructive pulmonary disease, pulmonary hypertension, interstitial lung fibrosis, rhinitis, aortic aneurysm, myocardial infarction, and stroke. In some aspects proliferative disorders treated by administering a compound described herein include, but are not limited to, cancer and noncancerous disorders, including, but not limited to, those involving the skin or lymphatic tissues. In further or alternative aspects metabolic disorders treated by administering a compound described herein include, but are not limited to, bone remodeling, loss or gain. In further or alternative aspects, conditions treated by the administration of a compound described herein are iatrogenic and increases in, or abnormal localization of, leukotrienes are induced by other therapies or medical or surgical procedures.

In further or alternative aspects, the methods, compounds, pharmaceutical compositions, and medicaments described herein are used to prevent the cellular activation of 5-lipoxygenase. In further or alternative aspects the methods, compounds, pharmaceutical compositions, and medicaments described herein are used to limit the formation of leukotrienes. In further or alternative aspects, the methods, compounds, pharmaceutical compositions, and medicaments comprise FLAP inhibitors disclosed herein for the treatment of asthma by (a) lowering the concentrations of leukotrienes in certain tissue(s) of the body or in the entire body of a patient, (b) modulating the activity of enzymes or proteins in a patient wherein such enzymes or proteins are involved in the leukotriene pathway such as, by way of example, 5-lipoxygenase-activating protein or 5-lipoxygenase, or (c) combining the effects of (a) and (b). In further or alternative aspects, the methods, compounds, pharmaceutical compositions, and medicaments described herein are used in combination with other medical treatments or surgical modalities.

In one aspect, provided herein are methods for reducing/inhibiting the leukotriene synthetic activity of 5-lipoxygenase-activating protein (FLAP) in a mammal comprising administering to the mammal at least once an effective amount of a compound having the structure of Formula (A), (B) or (C).

In a further or alternative embodiment, a “G” group (e.g. G¹, G², G³, G⁴) of Formula (A), (B) or (C) is any group that is used to tailor the physical and biological properties of the molecule. Such tailoring/modifications are achieved using groups which modulate acidity, basicity, lipophilicity, solubility and other physical properties of the molecule. The physical and biological properties modulated by such modifications to “G” include, by way of example only, solubility, in vivo absorption, and in vivo metabolism. In some instances, in vivo metabolism includes, by way of example only, controlling in vivo PK properties, off-target activities, potential toxicities associated with cypP450 interactions, drug-drug interactions, and the like. Further, modifications to “G” allow for the tailoring of the in vivo efficacy of the compound through the modulation of, by way of example, specific and non-specific protein binding to plasma proteins and lipids and tissue distribution in vivo. Additionally, such tailoring/modifications to “G” allow for the design of compounds selective for 5-lipoxygenase-activating protein over other proteins. In some embodiments, “G”, is L²⁰-Q, wherein L²⁰ is an enzymatically cleavable linker and Q is a drug, or an affinity moiety. In certain embodiments, the drug includes, by way of example only, leukotriene receptor antagonists and anti-inflammatory agents. In some embodiments, the leukotriene receptor antagonists include, but are not limited to, CysLT1/CysLT2 dual antagonists and CysLT1 antagonists. In certain embodiments, the affinity moiety allows for site specific binding and include, but are not limited to, antibodies, antibody fragments, DNA, RNA, siRNA, and ligands.

In further or alternative aspects, provided herein are methods for modulating, including reducing and/or inhibiting the activity of 5-lipoxygenase activating protein, directly or indirectly, in a mammal comprising administering to the mammal at least once an effective amount of at least one compound having the structure of Formula (A), (B) or (C).

In further or alternative aspects, provided herein are methods for modulating, including reducing and/or inhibiting, the activity of leukotrienes in a mammal, directly or indirectly, comprising administering to the mammal at least once an effective amount of at least one compound having the structure of Formula (A), (B) or (C).

In further or alternative aspects, provided herein are methods for treating leukotriene-dependent or leukotriene mediated conditions or diseases, comprising administering to the mammal at least once an effective amount of at least one compound having the structure of Formula (A), (B) or (C).

In further or alternative aspects, provided herein are methods for treating inflammation comprising administering to the mammal at least once an effective amount of at least one compound having the structure of Formula (A), (B) or (C).

In further or alternative aspects, provided herein are methods for treating respiratory diseases comprising administering to the mammal at least once an effective amount of at least one compound having the structure of Formula (A), (B) or (C). In further or alternative embodiments, the respiratory disease is asthma. In further or alternative embodiments, the respiratory disease includes, but is not limited to, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma,

In further or alternative aspects, provided herein are methods for treating chronic obstructive pulmonary disease comprising administering to the mammal at least once an effective amount of at least one compound having the structure of Formula (A), (B) or (C). In specific embodiments, chronic obstructive pulmonary disease includes, but is not limited to, chronic bronchitis or emphysema, pulmonary hypertension, interstitial lung fibrosis and/or airway inflammation and cystic fibrosis.

In further or alternative aspects, provided herein are methods for preventing increased mucosal secretion and/or edema in a disease or condition comprising administering to the mammal at least once an effective amount of at least one compound having the structure of Formula (A), (B) or (C).

In further or alternative aspects, provided herein are methods for treating vasoconstriction, atherosclerosis and its sequalae myocardial ischemia, myocardial infarction, aortic aneurysm, vasculitis and stroke comprising administering to the mammal an effective amount of a compound having the structure of Formula (A), (B) or (C).

In further or alternative aspects, provided herein are methods for treating organ reperfusion injury following organ ischemia and/or endotoxic shock comprising administering to the mammal at least once an effective amount of at least one compound having the structure of Formula (A), (B) or (C).

In further or alternative aspects, provided herein are methods for reducing the constriction of blood vessels in a mammal comprising administering to the mammal at least once an effective amount of at least one compound having the structure of Formula (A), (B) or (C).

In further or alternative aspects, provided herein are methods for lowering or preventing an increase in blood pressure of a mammal comprising administering to the mammal at least once an effective amount of at least one compound having the structure of Formula (A), (B) or (C).

In further or alternative aspects, provided herein are methods for preventing eosinophil and/or basophil and/or dendritic cell and/or neutrophil and/or monocyte recruitment comprising administering to the mammal at least once an effective amount of at least one compound having the structure of Formula (A), (B) or (C).

In further or alternative aspects, provided herein are methods for the prevention or treatment of abnormal bone remodeling, loss or gain, including diseases or conditions as, by way of example, osteopenia, osteoporosis, Paget's disease, cancer and other diseases comprising administering to the mammal at least once an effective amount of at least one compound having the structure of Formula (A), (B) or (C).

In further or alternative aspects, provided herein are methods for preventing ocular inflammation and allergic conjunctivitis, vernal keratoconjunctivitis, and papillary conjunctivitis comprising administering to the mammal at least once an effective amount of at least one having the structure of Formula (A), (B) or (C).

In further or alternative aspects, provided herein are methods for treating CNS disorders comprising administering to the mammal at least once an effective amount of at least one compound having the structure of Formula (A), (B) or (C). In some embodiments, CNS disorders include, but are not limited to, multiple sclerosis, Parkinson's disease, Alzheimer's disease, stroke, cerebral ischemia, retinal ischemia, post-surgical cognitive dysfunction, migraine, peripheral neuropathy/neuropathic pain, spinal cord injury, cerebral edema and head injury.

In further or alternative aspects, provided herein are methods for the treatment of cancer comprising administering to the mammal at least once an effective amount of at least one compound having the structure of Formula (A), (B) or (C). In certain embodiments, the type of cancer includes, but is not limited to, pancreatic cancer and other solid or hematological tumors.

In further or alternative aspects, provided herein are methods for treating endotoxic shock and septic shock comprising administering to the mammal at least once an effective amount of at least one compound having the structure of Formula (A), (B) or (C).

In further or alternative aspects, provided herein are methods for treating rheumatoid arthritis and osteoarthritis comprising administering to the mammal at least once an effective amount of at least one compound having the structure of Formula (A), (B) or (C).

In further or alternative aspects, provided herein are methods for preventing increased GI diseases comprising administering to the mammal at least once an effective amount of at least one compound having the structure of Formula (A), (B) or (C). Such diseases include, by way of example only, chronic gastritis, eosinophilic gastroenteritis, and gastric motor dysfunction.

In further or alternative aspects, provided herein are methods for treating kidney diseases comprising administering to the mammal at least once an effective amount of at least one compound having the structure of Formula (A), (B) or (C). Such diseases include, by way of example only, glomerulonephritis, cyclosporine nephrotoxicity renal ischemia reperfusion.

In further or alternative aspects, provided herein are methods for preventing or treating acute or chronic renal insufficiency comprising administering to the mammal at least once an effective amount of at least one compound having the structure of Formula (A), (B) or (C).

In further or alternative aspects, provided herein are methods for treating type II diabetes comprising administering to the mammal at least once an effective amount of at least one compound having the structure of Formula (A), (B) or (C).

In further or alternative aspects, provided herein are methods to diminish the inflammatory aspects of acute infections within one or more solid organs or tissues such as the kidney with acute pyelonephritis.

In further or alternative aspects, provided herein are methods for preventing or treating acute or chronic disorders involving recruitment or activation of eosinophils comprising administering to the mammal at least once an effective amount of at least one compound having the structure of Formula (A), (B) or (C).

In further or alternative aspects, provided herein are methods for preventing or treating acute or chronic erosive disease or motor dysfunction of the gastrointestinal tract caused by non-steroidal anti-inflammatory drugs (including selective or non-selective cyclooxygenase-1 or -2 inhibitors) comprising administering to the mammal at least once an effective amount of at least one compound having the structure of Formula (A), (B) or (C).

In further or alternative aspects, provided herein are methods for the prevention or treatment of rejection or dysfunction in a transplanted organ or tissue comprising administering to the mammal at least once an effective amount of at least one compound having the structure of Formula (A), (B) or (C).

In further or alternative aspects, provided herein are methods for treating inflammatory responses of the skin that include administering to the mammal at least once an effective amount of at least one compound having the structure of Formula (A), (B) or (C). Such inflammatory responses of the skin include, by way of example, dermatitis, contact dermatitis, eczema, urticaria, rosacea, and scarring. In further or alternative aspects are methods for reducing psoriatic lesions in the skin, joints, or other tissues or organs, comprising administering to the mammal an effective amount of a first compound having the structure of Formula (A), (B) or (C).

In further or alternative aspects, provided herein are methods for the treatment of cystitis, including, by way of example only, interstitial cystitis, which include administering to the mammal at least once an effective amount of at least one compound having the structure of Formula (A), (B) or (C).

In further or alternative aspects, provided herein are methods for the treatment of metabolic syndromes such as Familial Mediterranean Fever that include administering to the mammal at least once an effective amount of at least one compound having the structure of Formula (A), (B) or (C).

In further or alternative aspects, provided herein are methods to treat hepatorenal syndrome that include administering to the mammal at least once an effective amount of at least one compound having the structure of Formula (A), (B) or (C).

In further or alternative aspects, provided herein is the use of a compound of Formula (A), (B) or (C), in the manufacture of a medicament for treating an inflammatory disease or condition in an animal in which the activity of at least one leukotriene protein contributes to the pathology and/or symptoms of the disease or condition. In one embodiment of this aspect, the leukotriene pathway protein is 5-lipoxygenase-activating protein (FLAP). In certain embodiments of this aspect, the inflammatory disease or conditions are respiratory, cardiovascular, or proliferative diseases.

In any of the aforementioned aspects are certain embodiments in which administration is enteral, parenteral, or both, and wherein (a) the effective amount of the compound is systemically administered to the mammal; and/or (b) the effective amount of the compound is administered orally to the mammal; and/or (c) the effective amount of the compound is intravenously administered to the mammal; and/or (d) the effective amount of the compound administered by inhalation; and/or (e) the effective amount of the compound is administered by nasal administration; or and/or (f) the effective amount of the compound is administered by injection to the mammal; and/or (g) the effective amount of the compound is administered topically (dermal) to the mammal; and/or (h) the effective amount of the compound is administered by ophthalmic administration; and/or (i) the effective amount of the compound is administered rectally to the mammal.

In any of the aforementioned aspects are certain embodiments in which the mammal is a human, including embodiments wherein (a) the human has an asthmatic condition or one or more other condition(s) selected from the group consisting of allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, or seasonal asthma, or chronic obstructive pulmonary disease, or pulmonary hypertension or interstitial lung fibrosis. In any of the aforementioned aspects are certain embodiments in which the mammal is an animal model for pulmonary inflammation, examples of which are provided herein.

In any of the aforementioned aspects are certain embodiments that include single administrations of the effective amount of the compound, including further embodiments in which (i) the compound is administered once; (ii) the compound is administered to the mammal multiple times over the span of one day; (iii) continually; or (iv) continuously.

In any of the aforementioned aspects are certain embodiments that include multiple administrations of the effective amount of the compound, including further embodiments in which (i) the compound is administered in a single dose; (ii) the time between multiple administrations is every 6 hours; (iii) the compound is administered to the mammal every 8 hours. In some embodiments, the method comprises a drug holiday, wherein the administration of the compound is temporarily suspended or the dose of the compound being administered is temporarily reduced; at the end of the drug holiday, dosing of the compound is resumed. In certain embodiments, the length of the drug holiday varies from 2 days to 1 year.

In any of the aforementioned aspects involving the treatment of leukotriene dependent diseases or conditions are certain embodiments that include administering at least one additional agent. In certain embodiments, each agent is administered in any order. In some embodiments, the at least one additional agent includes, by way of example, an anti-inflammatory agent, a different compound having the structure of Formula (A), (B) or (C), a CysLT₁ receptor antagonist, or a CysLT₁/CysLT₂ dual receptor antagonist. In some embodiments, the CysLT₁ antagonist is selected from montelukast (Singulair®: [1-[[1-[3-[2-[(7-chloro-2-quinolyl)]vinyl]phenyl]-3-[2-(1-hydroxy-1-methyl-ethyl)phenyl]-propyl]sulfanylmethyl]cyclopropyl]acetic acid), zafirlukast (Accolate®: 3-[[2-methoxy-4-(o-tolylsulfonylcarbamoyl)phenyl]methyl]-1-methyl-1H-indol-5-yl]aminoformic acid cyclopentyl ester) or pranlukast (Onon™: 4-oxo-8-[p-(4-phenylbutyloxy)benzoylamino]-2-tetrazol-5-yl)-4H-1-benzopyran)

In certain embodiments, anti-inflammatory agents include, but are not limited to, non-steroidal anti-inflammatory drugs such as a cyclooxygenase inhibitor (COX-1 and/or COX-2), lipoxygenase inhibitors and steroids such as prednisone or dexamethasone. In further or alternative embodiments, the anti-inflammatory agent is selected from the group consisting of diclofenac (Arthrotec®), mesalamine (Asacol®, Ipocal®, Pentasa®, Salofalk®), antipyrine and benzocaine (Auralgan®), sulfasalazine (Azulfidine®), oxaprozin (Daypro®), mefenamic acid (Ponstan®), methylprednisolone (Solu-Medrol), indomethacin (Indocin®), rofecoxib (Vioxx®), celecoxib (Celebrex®), valdecoxib (Bextra®), etodolac (Lodine®), meloxicam (Mobic®), piroxicam (Feldene®), aspirin (Bayer®, Bufferin®), etoricoxib (Arcoxia®), lumiracoxib (Prexige®), ibuprofen (Advil®, Motrin®), diclofenac (Voltaren®), ketoprofen (Orudis®), meloxicam (Mobic®), nabumetone (Relafen®), naproxen (Aleve®, Naprosyn®), betamethasone (Celestone®), prednisolone, prednisone (Deltasone®), or any generic equivalent thereof.

In any of the aforementioned aspects involving the treatment of proliferative disorders, including cancer, are certain embodiments that include administering at least one additional agent selected from the group consisting of alemtuzumab, arsenic trioxide, asparaginase (pegylated or non-), bevacizumab, cetuximab, platinum-based compounds such as cisplatin, cladribine, daunorubicin/doxorabicin/idarubicin, irinotecan, fludarabine, 5-fluorouracil, gemtuzamab, methotrexate, paclitaxel (Taxol), temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, dronabinol.

In any of the aforementioned aspects involving the therapy of transplanted organs or tissues or cells are certain embodiments that include administering at least one additional agent selected from the group consisting of azathioprine, a corticosteroid, cyclophosphamide, cyclosporin, dacluzimab, mycophenolate mofetil, OKT3, rapamycin, tacrolimus, and thymoglobulin.

In any of the aforementioned aspects involving the therapy of interstitial cystitis are certain embodiments that include administering at least one additional agent selected from dimethylsulfoxide, omalizumab, and pentosan polysulfate.

In any of the aforementioned aspects involving the therapy of disorders of bone are certain embodiments that include administering at least one additional agent selected from the group consisting of minerals, vitamins, bisphosphonates, anabolic steroids, parathyroid hormone or analogs, and cathepsin K inhibitors, dronabinol.

In any of the aforementioned aspects involving the prevention or treatment of inflammation are certain embodiments that include: (a) monitoring inflammation in a mammal; (b) measuring bronchoconstriction in a mammal; (c) measuring eosinophil and/or basophil and/or dendritic cell and/or neutrophil and/or monocyte and/or lymphocyte recruitment in a mammal; (d) monitoring mucosal secretion in a mammal; (e) measuring mucosal edema in a mammal; (e) measuring levels of LTB₄ in the calcium ionophore-challenged blood of a mammal; (f) measuring levels of LTE₄ in the urinary excretion of a mammal; or (g) identifying a patient by measuring leukotriene-driven inflammatory biomarkers such as LTB₄, LTC₄, Il-6, CRP, SAA, MPO, EPO, MCP-1, MIP-α, sICAMs, Il-4, Il-13.

In any of the aforementioned aspects involving the prevention or treatment of leukotriene-dependent or leukotriene mediated diseases or conditions are certain embodiments that include identifying patients by screening for a leukotriene gene haplotype. In further or alternative embodiments the leukotriene gene haplotype is a leukotriene pathway gene, while in still further or alternative embodiments, the leukotriene gene haplotype is a 5-lipoxygenase-activating protein (FLAP) haplotype.

In any of the aforementioned aspects involving the prevention or treatment of leukotriene-dependent or leukotriene mediated diseases or conditions are certain embodiments that include identifying patients by monitoring the patient for either:
i) at least one leukotriene related inflammatory biomarker; or
ii) at least one functional marker response to a leukotriene modifying agent; or
iii) at least one leukotriene related inflammatory biomarker and at least one functional marker response to a leukotriene modifying agent.

In further or alternative embodiments, the leukotriene-related inflammatory biomarkers are selected from the group consisting of LTB₄, cysteinyl leukotrienes, CRP, SAA, MPO, EPO, MCP-1, MIP-α, sICAM, IL-6, IL-4, and IL-13, while in still further or alternative embodiments, the functional marker response is significant lung volume (FEV1).

In any of the aforementioned aspects involving the prevention or treatment of leukotriene-dependent or leukotriene mediated diseases or conditions are certain embodiments that include identifying patients by either:
i) screening the patient for at least one leukotriene gene SNP and/or haplotype including SNP's in intronic or exonic locations; or
ii) monitoring the patient for at least one leukotriene related inflammatory biomarker; or
ii) monitoring the patient for at least one functional marker response to a leukotriene modifying agent

In further or alternative embodiments, the leukotriene gene SNP or haplotype is a leukotriene pathway gene. In still further or alternative embodiments, the leukotriene gene SNP or haplotype is a 5-lipoxygenase-activating protein (FLAP) SNP or haplotype. In further or alternative embodiments, the leukotriene-related inflammatory biomarkers are selected from the group consisting of LTB₄, cysteinyl leukotrienes, CRP, SAA, MPO, EPO, MCP-1, MIP-α, sICAM, IL-6, IL-4, and IL-13, while in still further or alternative embodiments, the functional marker response is significant lung volume (FEV1).

In any of the aforementioned aspects involving the prevention or treatment of leukotriene-dependent or leukotriene mediated diseases or conditions are further embodiments that include identifying patients by at least two of the following:
i) screening the patient for at least one leukotriene gene SNP or haplotype;
ii) monitoring the patient for at least one leukotriene related inflammatory biomarker;
ii) monitoring the patient for at least one functional marker response to a leukotriene modifying agent.

In further or alternative embodiments, the leukotriene gene SNP or haplotype is a leukotriene pathway gene. In still further or alternative embodiments, the leukotriene gene SNP or haplotype is a 5-lipoxygenase-activating protein (FLAP) SNP or haplotype. In further or alternative embodiments, the leukotriene-related inflammatory biomarkers are selected from the group consisting of LTB₄, cysteinyl leukotrienes, CRP, SAA, MPO, EPO, MCP-1, MIP-α, sICAM, IL-6, IL-4, and IL-13, while in still further or alternative embodiments, the functional marker response is significant lung volume (FEV1).

In any of the aforementioned aspects involving the prevention or treatment of leukotriene-dependent or leukotriene mediated diseases or conditions are further embodiments that include identifying patients by:
i) screening the patient for at least one leukotriene gene SNP or haplotype; and
ii) monitoring the patient for at least one leukotriene related inflammatory biomarker; and
ii) monitoring the patient for at least one functional marker response to a leukotriene modifying agent.

In further or alternative embodiments, the leukotriene gene SNP or haplotype is a leukotriene pathway gene. In still further or alternative embodiments, the leukotriene gene SNP or haplotype is a 5-lipoxygenase-activating protein (FLAP) SNP or haplotype. In further or alternative embodiments, the leukotriene-related inflammatory biomarkers are selected from the group consisting of LTB₄, cysteinyl leukotrienes, CRP, SAA, MPO, EPO, MCP-1, MIP-α, sICAM, IL-6, IL-4, and IL-13, while in still further or alternative embodiments, the functional marker response is significant lung volume (FEV1).

In further or alternative aspects is the prevention or treatment of leukotriene-dependent or leukotriene mediated diseases or conditions that include administering to a patient an effective amount of a FLAP modulator, wherein the patients has been identified using information obtained by:
i) screening the patient for at least one leukotriene gene SNP or haplotype; and
ii) monitoring the patient for at least one leukotriene related inflammatory biomarker; and
ii) monitoring the patient for at least one functional marker response to a leukotriene modifying agent.

In further or alternative embodiments, the FLAP modulator is a FLAP inhibitor. In further or alternative embodiments, the leukotriene gene SNP or haplotype is a leukotriene pathway gene. In still further or alternative embodiments, the leukotriene gene SNP or haplotype is a 5-lipoxygenase-activating protein (FLAP) SNP or haplotype. In further or alternative embodiments, the leukotriene-related inflammatory biomarkers are selected from the group consisting of LTB₄, cysteinyl leukotrienes, CRP, SAA, MPO, EPO, MCP-1, MIP-α, sICAM, IL-6, IL-4, and IL-13, while in still further or alternative embodiments, the functional marker response is significant lung volume (FEV1). In further or alternative embodiments, the information obtained from the three diagnostic methods may be used in an algorithm in which the information is analyzed to identify patients in need of treatment with a FLAP modulator, the treatment regimen, and the type of FLAP modulator used.

In any of the aforementioned aspects the leukotriene-dependent or leukotriene mediated diseases or conditions include, but are not limited to, asthma, chronic obstructive pulmonary disease, pulmonary hypertension, interstitial lung fibrosis, rhinitis, arthritis, allergy, inflammatory bowel disease, adult respiratory distress syndrome, myocardial infarction, aneurysm, stroke, cancer, and endotoxic shock.

In further or alternative embodiments, compounds provided herein are administered to a human.

In further or alternative embodiments, compounds provided herein are orally administered.

In further or alternative embodiments, compounds provided herein are used for inhibiting the activity of FLAP. In further or alternative embodiments, compounds provided herein are used for inhibiting the activity of FLAP or for the treatment of a disease or condition that would benefit from inhibition of FLAP activity.

In further or alternative embodiments, compounds provided herein are used for the formulation of a medicament for the inhibition of FLAP activity.

Articles of manufacture, which include packaging material, a compound described herein, such as, for example, a compound of Formula (A), (B) or (C), which is effective for modulating the activity of 5-lipoxygenase activating protein (FLAP), or for treatment, prevention or amelioration of one or more symptoms of a leukotriene dependent or leukotriene-mediated disease or condition or a 5-lipoxygenase activating protein dependent or 5-lipoxygenase activating protein-mediated disease or condition, within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for modulating the activity of 5-lipoxygenase activating protein, or for treatment, prevention or amelioration of one or more symptoms of a leukotriene dependent or leukotriene-mediated disease or condition or a FLAP dependent or FLAP-mediated disease or condition.

Other objects, features and advantages of the methods, compounds, and compositions described herein will become apparent from the following detailed description. It should be understood, however, that the detailed description and the specific examples, while indicating specific embodiments, are given by way of illustration only.

### BRIEF DESCRIPTION OF THE FIGURES

FIG. 1 presents illustrative schemes for the syntheses of compounds described herein.

FIG. 2 presents illustrative schemes for the syntheses of compounds described herein.

FIG. 3 presents illustrative schemes for the syntheses of compounds described herein.

FIG. 4 presents illustrative schemes for the syntheses of compounds described herein.

FIG. 5 presents illustrative schemes for the syntheses of compounds described herein.

FIG. 6 presents illustrative examples of compounds described herein.

FIG. 7 presents illustrative examples of compounds described herein.

FIG. 8 presents illustrative examples of compounds described herein.

FIG. 9 present an illustrative scheme for the treatment of patients using the compounds and methods described herein.

FIG. 10 present an illustrative scheme for the treatment of patients using the compounds and methods described herein.

FIG. 11 present an illustrative scheme for the treatment of patients using the compounds and methods described herein.

### DETAILED DESCRIPTION

Leukotrienes (LTs) are potent contractile and inflammatory mediators produced by release of arachidonic acid from cell membranes. Arachidonic acid is converted to leukotrienes by the action of 5-lipoxygenase, 5-lipoxygenase-activating protein, LTA₄ hydrolase and LTC₄ synthase. The leukotriene synthesis pathway, or 5-lipoxygenase pathway, involves a series of enzymatic reactions in which arachidonic acid is converted to leukotriene LTB₄, or the cysteinyl leukotrienes, LTC₄, LTD₄, and LTE₄. The pathway occurs mainly at the nuclear envelope. Protein components dedicated to the leukotriene synthesis pathway include 5-lipoxygenase (5-LO), 5-lipoxygenase-activating protein, LTA₄ hydrolase, and LTC₄ synthase. Leukotrienes are synthesized directly from arachidonic acid by different cells including eosinophils, neutrophils, basophils, lymphocytes, macrophages, monocytes and mast cells. Excess LTA₄, for example from an activated neutrophil, may enter a cell by a transcellular pathway. Most cells in the body have LTA₄ hydrolase and so can produce LTB₄. Platelets and endothelial cells have LTC₄ synthase and so can make LTC₄ when presented with LTA₄ by a transcellular pathway. Wood, J W et al, J. Exp. Med., 178: 1935-1946, (1993); Peters-Golden, Am. J. Respir. Crit. Care Med. 157:S227-S232 (1998); Drazen, et al., ed. Five-Lipoxygenase Products in Asthma, Lung Biology in Health and Disease Series, Vol. 120, Chapters 1, 2, and 7, Marcel Dekker, Inc. NY (1998); Samuelsson et al, Science, 220, 568-575 (1983); and Peters-Golden, “Cell Biology of the 5-Lipoxygenase Pathway” Am J Respir Crit Care Med 157:S227-S232 (1998) are hereby incorporated by reference for such disclosure.

Arachidonic acid is a polyunsaturated fatty acid and is present mainly in the membranes of the body's cells. Upon presentation of inflammatory stimuli from the exterior of the cell, calcium is released and binds to phospholipase A₂ (PLA2) and 5-LO. Cell activation results in the translocation of PLA₂ and 5-LO from the cytoplasm to the endoplasmic reticulum and/or nuclear membranes, where in the presence of FLAP, the released arachidonic acid is converted, via a 5-HPETE intermediate, to the epoxide LTA₄. Depending on the cell type, the LTA₄ is immediately converted to LTC₄, by the nuclear-bound LTC₄ synthase or to LTB₄ by the action of cytosolic LTA₄ hydrolase. LTB₄ is exported from cells by an as yet uncharacterized transporter and may activate other cells, or the cell it was made in, via high affinity binding to one of two G protein-coupled receptors (GPCRs), namely BLT₁R or BLT₂R. LTC₄ is exported to the blood via the MRP-1 anion pump and rapidly converted to LTD₄ by the action of γ-glutamyl transpeptidase and LTD₄ is then converted to LTE₄ by the action of dipeptidases. LTC₄, LTD₄ and LTE₄ are collectively referred to as the cysteinyl leukotrienes (or as slow reacting substance of anaphylaxis, SRS-A). The cysteinyl leukotrienes activate other cells, or the cells they are made in, via high affinity binding to one of two GPCRs, namely CysLT₁R or CysLT₂R. CysLT₁ receptors are found in the human airway eosinophils, neutrophils, macrophages, mast cells, B-lymphocytes and smooth muscle and induce bronchoconstriction. CysLT₂ receptors are located in human airway eosinophils, macrophages, mast cells the human pulmonary vasculature. Zhu et al, Am J Respir Cell Mol Biol Epub Aug. 25 (2005); and Figueroa et al, Clin Exp Allergy 33:1380-13880 (2003) are hereby incorporated by reference for such disclosure.

Leukotrienes are involved in various diseases. Leukotrienes produce marked inflammatory responses in human skin. Evidence for the involvement of leukotrienes in a human disease is found in psoriasis, in which leukotrienes are detected in psoriatic lesions. Busse, Clin. Exp. Allergy 26:868-79 (1996); O'Byrne, Chest 111(Supp. 2): 27S-34S (1977); Sheftell, F. D., et al., Headache, 40:158-163 (2000); Klickstein et al., J. Clin. Invest., 66:1166-1170 (1950); Davidson et al., Ann. Rheum. Dis., 42:677-679 (1983); and Kragballe et al., Arch. Dermatol., 119:548-552 (1983) are hereby incorporated by reference for such disclosure.

For example, inflammatory responses include three types of changes in the local blood vessels. The primary change is an increase in vascular diameter, which results in an increase in local blood flow and leads to an increased temperature, redness and a reduction in the velocity of blood flow, especially along the surfaces of small blood vessels. The second change is the activation of endothelial cells lining the blood vessel to express adhesion molecules that promote the binding of circulating leukocytes. The combination of slowed blood flow and induced adhesion molecules allows leukocytes to attach to the endothelium and migrate into the tissues, a process known as extravasation. These changes are initiated by cytokines and leukotrienes produced by activated macrophages. Once inflammation has begun, the first cells attracted to the site of infection are generally neutrophils. They are followed by monocytes, which differentiate into more tissue macrophages. In the latter stages of inflammation, other leukocytes, such as eosinophils and lymphocytes also enter the infected site. The third major change in the local blood vessels is an increase in vascular permeability. Instead of being tightly joined together, the endothelial cells lining the blood vessel walls become separated, leading to exit of fluid and proteins from the blood and their local accumulation in the tissue. Janeway, et al., Immunobiology: the immune system in health and disease, 5th ed., Garland Publishing, New York (2001) is hereby incorporated by reference for such disclosure.

LTB₄ produces relatively weak contractions of isolated trachea and lung parenchyma, and these contractions are blocked in part by inhibitors of cyclooxygenase, indicating that the contractions are secondary to the release of prostaglandins. However, LTB₄ is a potent chemotactic agent for eosinophils and progenitors of mast cells and the LTB₄ receptor BLT1−/− knockout mouse is protected from eosinophilic inflammation and T-cell mediated allergic airway hyperreactivity. Miyahara et al. J Immunol 174:4979-4784; and Weller et al. J Exp Med 201:1961-1971 (2005) are hereby incorporated by reference for such disclosure.

Leukotrienes C₄ and D₄ are potent smooth muscle contractile agents, promoting bronchoconstriction in a variety of species, including humans. These compounds have profound hemodynamic effects, constricting coronary blood vessels, and resulting in a reduction of cardiac output efficiency. Leukotrienes also act as vasoconstrictors, however, marked differences exist for different vascular beds. In some instances, leukotrienes contribute to cardiac reperfusion injury following myocardial ischemia. LTC₄ and LTD₄ directly increase vascular permeability. In one aspect, increased vascular permeability may be achieved by promoting retraction of capillary endothelial cells via activation of the CysLT₂ receptor and possibly other as yet undefined CysLT receptors. LTB₄ enhances atherosclerotic progression in two atherosclerotic mouse models, namely low density receptor lipoprotein receptor deficient (LDLr−/−) mice and apolipoprotein E-deficient (ApoE−/−) mice. LTB₄ also increases human monocyte chemoattractant protein (MCP-1), an enhancer of atherosclerotic progression. Marone et al., in Biology of Leukotrienes, ed. By R. Levi and R. D. Krell, Ann. New York Acad. Sci. 524:321-333 (1988); Barst and Mullane, Eur. J. Pharmacol., 114: 383-387 (1985); Sasaki et al., Cardiovasc. Res., 22: 142-148 (1988); Lotzer et al. Arterioscler Thromb Vase Biol 23: e32-36 (2003); Aiello et al, Arterioscler Thromb Vase Biol 22:443-449 (2002); Subbarao et al, Arterioscler Thromb Vase Biol 24:369-375 (2004); Heller et al. Circulation 112:578-586 (2005); Huang et al. Aterioscler Thromb Vase Biol 24:1783-1788 (2004); and Dahlen et al., Nature, 288:484-486 (1980) are hereby incorporated by reference for such disclosure.

The role of FLAP in the leukotriene synthesis pathway is significant because FLAP in concert with 5-lipoxygenase performs the first step in the pathway for the synthesis of leukotrienes. Therefore, the leukotriene synthesis pathway provides a number of targets for compounds useful in the treatment of leukotriene-dependent or leukotriene mediated diseases or conditions, including, by way of example, vascular and inflammatory disorders, proliferative diseases, and non-cancerous disorders.

Leukotriene-dependent or leukotriene mediated conditions treated using the methods, compounds, pharmaceutical compositions and medicaments described herein, include, but are not limited to, bone diseases and disorder, cardiovascular diseases and disorders, inflammatory diseases and disorders, dermatological diseases and disorders, ocular diseases and disorders, cancer and other proliferative diseases and disorders, respiratory diseases and disorder, and non-cancerous disorders.

Leukotrienes contribute to the inflammation of the airways of patients with asthma. CysLT₁ receptor antagonists such as montelukast (Singulair®) are efficacious in asthma and allergic rhinitis. CysLT₁R antagonists pranlukast (Onon™) and zafirlukast (Accolate™) have also been shown to be efficacious in asthma. Reiss et al. Arch Intern Med 158:1213-1220 (1998); and Phillip et al. Clin Exp Allergy 32:1020-1028 (2002) are hereby incorporated by reference for such disclosure.

A number of drugs inhibit leukotriene formation, including the 5-lipoxygenase inhibitor zileuton (Zyflo®) that has efficacy in asthma. The 5-lipoxygenase inhibitor ZD2138 (6-[(3-fluoro-5-[4-methoxy-3,4,5,6-tetrahydro-2H-pyran-4-yl])phenoxy-methyl]-1-methyl-2-quinolone) has efficacy in inhibiting the fall of FEV1 resulting from aspirin-induced asthma. The following leukotriene synthesis inhibitors have efficacy in asthma: MK-0591 (2-((5-((quinolin-2-yl)methoxy)-1-(4-chlorobenzyl)-3-(tert-butylthio)-1H-indol-2-yl)methyl)-2-methylpropanoic acid), a specific inhibitor of 5-lipoxygenase-activating protein (FLAP), MK-886 (2-((1-(4-chlorobenzyl)-3-(tert-butylthio)-5-isopropyl-1H-indol-2-yl)methyl)-2-methylpropanoic acid), a specific inhibitor of 5-lipoxygenase-activating protein (FLAP), and BAY X1005 ((R)-2-[4-(quinolin-2-yl-methoxy)phenyl]-2-cyclopentyl acetic acid), a specific inhibitor of 5-lipoxygenase-activating protein (FLAP). Israel et al. Ann Intern Med 119:1059-1066 (1993); Nasser et al, Thorax, 49; 749-756 (1994); Brideau, et al., Ca. J. Physiol. Pharmacol. 70:799-807 (1992); Friedman et al. Am Rev Respir Dis., 147: 839-844 (1993); and Fructmann et al, Agents Actions 38: 188-195 (1993) are hereby incorporated by reference for such disclosure.

FLAP inhibition will decrease LTB₄ from monocytes, neutrophils and other cells involved in vascular inflammation and thereby decrease atherosclerotic progression. The FLAP inhibitor MK-886 decreases the postangioplasty vasoconstrictive response in a porcine carotid injury model. MK-886 also suppresses femoral artery intimal hyperplasia in a rat photochemical model of endothelial injury. The 5-lipoxygenase inhibitor zileuton has been shown to reduce renal ischemia in a mouse model. Provost et al. Brit J Pharmacol 123: 251-258 (1998); Kondo et al. Thromb Haemost 79:635-639 (1998); and Nimesh et al. Mol Pharm 66:220-227 (2004) are hereby incorporated by reference for such disclosure.

FLAP modulators, inhibitors and/or antagonists are used for the treatment of a variety of diseases or conditions, including, by way of non-limiting example, (i) inflammation; (ii) respiratory diseases including asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma; (iii) chronic obstructive pulmonary disease, including chronic bronchitis or emphysema, pulmonary hypertension, interstitial lung fibrosis and/or airway inflammation and cystic fibrosis; (iv) increased mucosal secretion and/or edema in a disease or condition; (v) vasoconstriction, atherosclerosis and its sequalae myocardial ischemia, myocardial infarction, aortic aneurysm, vasculitis and stroke; (vi) organ reperfusion injury following organ ischemia and/or endotoxic shock; (vii) constriction of blood vessels; (viii) an increase in blood pressure; (ix) eosinophil and/or basophil and/or dendritic cell and/or neutrophil and/or monocyte recruitment; (x) abnormal bone remodeling, loss or gain, including osteopenia, osteoporosis, Paget's disease, cancer and other diseases; (xi) ocular inflammation and allergic conjunctivitis, vernal keratoconjunctivitis, and papillary conjunctivitis; (xii) CNS disorders, including, but are not limited to, multiple sclerosis, Parkinson's disease, Alzheimer's disease, stroke, cerebral ischemia, retinal ischemia, post-surgical cognitive dysfunction, migraine; (xiii) peripheral neuropathy/neuropathic pain, spinal cord injury, cerebral edema and head injury; (xiv) cancer, including, but not limited to, pancreatic cancer and other solid or hematological tumors; (xv) endotoxic shock and septic shock; (xvi) rheumatoid arthritis and osteoarthritis; (xvii) increased GI diseases, including, by way of example only, chronic gastritis, eosinophilic gastroenteritis, and gastric motor dysfunction; (xviii) kidney diseases, including, by way of example only, glomerulonephritis, cyclosporine nephrotoxicity renal ischemia reperfusion; (xix) acute or chronic renal insufficiency; (xx) type II diabetes; (xxi) inflammatory aspects of acute infections within one or more solid organs or tissues such as the kidney with acute pyelonephritis; (xxii) acute or chronic disorders involving recruitment or activation of eosinophils; (xxiii) acute or chronic erosive disease or motor dysfunction of the gastrointestinal tract caused by non-steroidal anti-inflammatory drugs (including selective or non-selective cyclooxygenase-1 or -2 inhibitors); (xxiv) metabolic syndromes, including, by way of example only, Familial Mediterranean Fever; and (xxv) hepatorenal syndrome. Leff A R et al., Ann Allergy Asthma Immunol 86(Suppl 1):4-8 (2001); Riccioni G, et al., Ann Clin Lab Sci. 34(4):379-870 (2004); Riccioni et al, Ann. Clin. Lab. Sci., v34, 379-387 (2004); Kostikas K et al., Chest 127:1553-9 (2004); Shahab R et al., J Laryngol Otol., 118; 500-7 (2004); Jala et al, Trends in Immunol., v25, 315-322 (2004); Mehrabian et al, Curr. Opin. Lipidol., v14, 447-457 (2003); Matsui N, et al., Planta Med. 71(4717-20 (2005); Stanke-Labesque F et al., Br J. Pharmacol. 140(1):186-94 (2003); Stanke-Labesque F et al., Br J. Pharmacol. 140(1):186-94 (2003); Walch L, et al., Br J. Pharmacol. 137(8):1339-45 (2002); Miyahara N, et al., Immunol. 15; 174(8):4979-84 (2005); Anderson G I, et al., Biomed Mater Res. 58(4):406-140 (2001); Lambiase et al, Arch. Opthalmol., v121, 615-620 (2003); de Souza Carvalho D, et al., Headache. 42(10):1044-7 (2002); Sheftell F, et al., Headache. 40(2):158-63 (2000); Akpek E A, et al., Spine. 24(2):128-32 (1999); Poff and Balazy, Curr. Drug Targets Inflamm. Allergy, v3, 19-33 (2004); Steele et al, Cancer Epidemiology & Prevention, v8, 467-483 (1999); Leite M S, et al., Shock. 23(2):173-8 (2005); Allen R, et al., Ann Rheum Dis. 63(2):170-6 (2004); Gyomber et al, J Gastroenterol Hepatol., v11, 922-927 (1996); Quack I et al BMC Gastroenterol v18,24 (2005); Cuzzocrea S, et al., Lab Invest. 85(6):808-22 (2005); Guasch et al Kidney Int., v56, 261-267; Butterly et al, v 57, 2586-2593 (2000); Guasch A et al. Kidney Int. 56:261-7 (1999); Butterly D W et al. Kidney Int. 57:2586-93 (2000); Maccarrone M, et al., J Am Soc Nephroi. 10:1991-6 (1999); Valdivielso et al, v16, 85-94 (2003); Parlapiano C, et al., Diabetes Res Clin Pract. 46(1):43-5 (1999); Tardif M, et al., L-651,392, Antimicrob Agents Chemother. 38(7):1555-60 (1994); Quack I, et al. BMC Gastroenterol., 5:24 (2005); Marusova I B, et al., Eksp Klin Farmakol, 2002; 65:16-8 and Gyomber E, et al., J. Gastroenterol. Hepatol., 1996, 11, 922-7) and Martin St et al., Eur J Gastroenterol. Hepatol., 17:983-6 (2005); Bentancur A G, et al., Clin Exp Rheumatol. 22(4 Suppl 34):S56-8 (2004); and Capella G L., Prostaglandins Leukot Essent Fatty Acids. 68(4):263-5 (2003) are hereby incorporated by reference for such disclosure.

Several inhibitors of FLAP have been described (Gillard et al, Can. J. Physiol. Pharmacol., 67, 456-464, 1989; Evans et al, Molecular Pharmacol., 40, 22-27, 1991; Brideau et al, Can. J. Physiol. Pharmacol., Musser et al, J. Med. Chem., 35, 2501-2524, 1992; Brooks et al., J. Med. Chem. 1996, vol. 39, no. 14, pp 2629-2654; Steinhilber, Curr. Med. Chem. 6(1):71-85, 1999; Riendeau, Bioorg Med Chem. Lett., 15(14):3352-5, 2005; Flamand, et al., Mod. Pharmacol. 62(2):250-6, 2002; Folco, et al., Am. J. Respir. Crit. Care Med. 161(2 Pt 2):S112-6, 2000; Hakonarson, JAMA, 293(18):2245-56, 2005).

The development and testing of FLAP inhibitors which are effective either alone or in combination with other drugs, and which result in minimal negative side effects are beneficial for treating leukotriene-dependent or leukotriene mediated diseases or conditions. In some embodiments, inhibitors of the leukotriene synthesis pathway described herein target any one or more step of the pathway to inhibit, prevent or reduce the formation of leukotrienes. In some embodiments, leukotriene synthesis inhibitors, by way of non-limiting example, inhibit at the level of FLAP and/or 5-LO, thus minimizing the formation of various products in the leukotriene pathway and decreasing the amounts of such compounds available in the cell. In various embodiments, leukotriene synthesis inhibitors are identified based on their ability to bind to proteins in the leukotriene synthesis pathway. For example, in specific embodiments, FLAP inhibitors are identified based on their binding to FLAP.

### Compounds

In certain embodiments, provided herein are compounds of Formula (A), pharmaceutically acceptable salts, pharmaceutically acceptable N-oxides, pharmaceutically active metabolites, pharmaceutically acceptable prodrugs, and pharmaceutically acceptable solvates thereof. In some embodiments, compounds of Formula (A) antagonize or inhibit FLAP. In certain embodiments, compounds of Formula (A) are used to treat patients suffering from leukotriene-dependent or leukotriene mediated conditions or diseases, including, but not limited to, asthma, myocardial infarction, cancer, and inflammatory conditions.

Formula (A) is as follows:

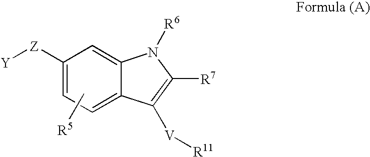

wherein:
Z is selected from among —[C(R¹)₂]ₘ—[C(R²)₂]ₙ, —[C(R²)₂]ₙ—[C(R)₂]ₘ—O—, —O—[C(R¹)₂]ₘ—[C(R²)₂]ₙ—, —[C(R²)₂]ₙ—O—[C(R¹)₂]ₙ—, and —[C(R¹)₂]ₙ—O—[C(R²)₂]ₙ—;
   each R¹ is independently H, —CF₃, or an optionally substituted C₁-C₄alkyl; or
   two R¹ on the same carbon taken together with the carbon to which they are attached form a carbonyl (C═O);
   each R² is independently H, —OH, —OMe, —CF₃, or an optionally substituted C₁-C₄alkyl; or
   each R² is (L^{s}R^{s}); or
   two R² on the same carbon taken together with the carbon to which they are attached form a carbonyl (C═O);
   m is 0, 1 or 2;
   each n is independently 0, 1, 2, or 3;
Y is H, -(substituted or unsubstituted C₁-C₆alkyl), -(substituted or unsubstituted C₃-C₈cycloalkyl), -(substituted or unsubstituted aryl), -(substituted or unsubstituted heteroaryl), or -(substituted or unsubstituted heterocycloalkyl);
where if Y is substituted, then each substitutent of Y is independently (L^{s}R^{s}),
   each L^{s} is independently selected from among a bond, —O—, —C(═O)—, —S—, —S(═O)—, —S(═O)₂—, —NHC(═O)—, —C(═O)NH—, S(═O)₂NH—, —NHS(═O)₂, —OC(═O)NH—, —NHC(═O)O—, —OC(═O)O—, —NHC(═O)NH—, —C(═O)O—, —OC(═O)—, (substituted or unsubstituted C₁-C₆alkyl), (C₂-C₆alkenyl), (C₁-C₆fluoroalkyl), (substituted or unsubstituted heteroaryl), (substituted or unsubstituted aryl), and (substituted or unsubstituted heterocycloalkyl);
   each R^{s} is independently selected from among H, halogen, —N(R⁹)₂, —CN, —NO₂, —S(═O)₂NH₂, (substituted or unsubstituted C₁-C₆alkyl), (substituted or unsubstituted C₃-C₈cycloalkyl), (C₁-C₆fluoroalkyl), (substituted or unsubstituted aryl), (substituted or unsubstituted heteroaryl), and (substituted or unsubstituted C₁-C₆heteroalkyl);
R⁵ is H, halogen, substituted or unsubstituted C₁-C₆alkyl, or substituted or unsubstituted —O—(C₁-C₆alkyl);
R⁶ is H, -L²-(substituted or unsubstituted C₁-C₆alkyl), -L²-(substituted or unsubstituted C₃-C₈cycloalkyl), -L²-(substituted or unsubstituted C₂-C₆alkenyl), -L²-(substituted or unsubstituted C₅-C₈cycloalkenyl), -L²-(substituted or unsubstituted heterocycloalkyl), -L²-(substituted or unsubstituted heteroaryl), or -L²-(substituted or unsubstituted aryl); and
   L² is a bond, —S(═O)₂—, —C(═O)—, or -(substituted or unsubstituted C₁-C₆alkyl)-;
R⁷ is L³-X-L⁴-G¹, wherein,
   L³ is a substituted or unsubstituted C₁-C₆alkyl;
   X is a bond, —O—, —C(═O)—, —CR⁹(OR⁹)—, —S—, —S(═O)—, —S(═O)₂—, —NR⁹—, —NR⁹C(═O)—, —C(═O)NR⁹—, aryl, heteroaryl or —NR⁹C(═O)NR⁹—;
   L⁴ is a bond, or a substituted or unsubstituted C₁-C₆alkyl;
   G¹ is H, tetrazolyl, —NHS(═O)₂R⁸, S(═O)₂N(R⁹)₂, —OR⁹, —C(═O)CF₃, —C(═O)NHS(═O)₂R⁸, —S(═O)₂NHC(═O)R⁹, —CN, —N(R⁹)₂, —N(R⁹)C(═O)R⁸, —C(═NR¹⁰)N(R⁹)₂, —NR⁹C(═NR¹⁰)N(R⁹)₂, —NR⁹C(═CHR¹⁰)N(R⁹)₂, —C(═O)NR⁹C(═NR¹⁰)N(R⁹)₂, —C(═O)NR⁹C(═CHR¹⁰)N(R⁹)₂, —CO₂R⁹, —C(═O)R⁹, —CON(R⁹)₂, —SR⁸, —S(═O)R⁸, —S(═O)₂R⁸, -L⁵-(substituted or unsubstituted C₁-C₆alkyl), -L⁵-(substituted or unsubstituted C₂-C₆alkenyl), -L⁵-(substituted or unsubstituted heteroaryl), or -L⁵-(substituted or unsubstituted aryl);
      L⁵ is —OC(═O)O—, —NHC(═O)NH—, —NHC(═O)O, —OC(═O)NH—, —NHC(═O), —C(═O)NH, —C(═O)O—, or —OC(═O)—;
   or G¹ is W-G², where
      W is a (substituted or unsubstituted aryl), (substituted or unsubstituted heterocycloalkyl), or (substituted or unsubstituted heteroaryl); and
      G² is H, tetrazolyl, —NHS(═O)₂R⁸, S(═O)₂N(R⁹)₂, OH, —OR⁸, —C(═O)CF₃, —C(═O)NHS(═O)₂R⁸, —S(═O)₂NHC(O)R⁹, CN, N(R⁹)₂, —N(R⁹)C(═O)R⁸, —C(═NR¹⁰)N(R⁹)₂, —NR⁹C(═NR¹⁰)N(R⁹)₂, —NR⁹C(═CHR¹⁰)N(R⁹)₂, —C(═O)NR⁹C(═NR¹⁰)N(R⁹)₂, —C(═O)NR⁹C(═CHR¹⁰)N(R⁹)₂, —CO₂R⁹, —C(═O)R⁹, —CON(R⁹)₂, —SR⁸, —S(═O)R⁸, or —S(═O)₂R⁸;
      each R⁸ is independently selected from among (substituted or unsubstituted C₁-C₄alkyl), (substituted or unsubstituted C₃-C₈cycloalkyl), (substituted or unsubstituted phenyl), (substituted or unsubstituted heteroaryl), and (substituted or unsubstituted benzyl);
      each R⁹ is independently selected from among H, (substituted or unsubstituted C₁-C₄alkyl), (substituted or unsubstituted C₃-C₈cycloalkyl), (substituted or unsubstituted phenyl), (substituted or unsubstituted heteroaryl), and (substituted or unsubstituted benzyl); or
      two R⁹ groups can together form a 5-, 6-, 7-, or 8-membered heterocyclic ring; or
      R⁸ and R⁹ can together form a 5-, 6-, 7-, or 8-membered heterocyclic ring; and
      each R¹⁰ is independently selected from H, —S(═O)₂R⁸, —S(═O)₂NH₂, —C(O)R⁸, —CN, —NO₂, heteroaryl, or heteroalkyl;
R¹¹ is L⁷-L¹⁰-G³, wherein
   L⁷ is a bond, or (substituted or unsubstituted C₁-C₆alkyl);
   L¹⁰ is a bond, (substituted or unsubstituted C₁-C₆alkyl), (substituted or unsubstituted C₃-C₈cycloalkyl), (substituted or unsubstituted heteroaryl), (substituted or unsubstituted aryl), or (substituted or unsubstituted heterocycloalkyl);
   G³ is —OR⁹, —C(═O)R⁹, —C(═O)OR⁹, —S(═O)R⁸, —S(═O)₂R⁸, —N(R⁹)₂, tetrazolyl, —NHS(═O)₂R⁸, —S(═O)₂N(R⁹)₂, —C(═O)NHS(═O)₂R⁸, —S(═O)₂NHC(═O)R⁸, —C(═O)N(R⁹)₂, —NR⁹C(═O)R⁸, —C(R⁹)₂C(═O)N(R⁹)₂, —C(═NR¹⁰)N(R⁹)₂, —NR⁹(═NR¹⁰)N(R⁹)₂, —NR⁹C(═CHR¹⁰)N(R⁹)₂, -L⁸-(substituted or unsubstituted C₁-C₆alkyl), -L⁸-(substituted or unsubstituted C₂-C₆alkenyl), -L⁸-(substituted or unsubstituted heteroaryl), or -L⁸-(substituted or unsubstituted aryl);
      L⁸ is —O—, —C(═O)—, —S—, —S(═O)—, —S(═O)₂—, —NH—, —NHC(═O)O—, —NHC(═O)NH—, —OC(═O)O—, —OC(═O)NH—, —NHC(═O)—, —C(═O)NH—, —C(═O)O—, or —OC(═O)—;
   or G³ is W⁴-G⁴, wherein
      W⁴ is (substituted or unsubstituted C₁-C₆alkyl), (substituted or unsubstituted heterocycloalkyl), (substituted or unsubstituted aryl) or a (substituted or unsubstituted heteroaryl); and
   G⁴ is H, halogen, —CN, —NO₂, —N₃, —CF₃, —OCF₃, C₁-C₆alkyl, C₃-C₈cycloalkyl, C₁-C₆fluoroalkyl, tetrazolyl, —NHS(═O)₂R⁸, —S(═O)₂N(R⁹)₂, —OH, —OR^{B}, —C(═O)CF₃, —C(O)NHS(═O)₂R⁸, —S(═O)₂NHC(O)R⁸, —CN, —N(R⁹)₂, —N(R⁹)C(O)R⁸, —C(═NR¹⁰)N(R⁹)₂, —NR⁹C(═CHR¹⁰)N(R⁹)₂, —C(O)NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂R⁹, —C(═O)R⁹, —CON(R⁹)₂, —SR⁸, —S(═O)R⁸, —S(═O)₂R⁸, -L⁹-(substituted or unsubstituted C₁-C₆alkyl), -L⁹-(substituted or unsubstituted C₂-C₆alkenyl), -L⁹-(substituted or unsubstituted C₁-C₆heteroalkyl), -L⁹-(substituted or unsubstituted heteroaryl), -L⁹-(substituted or unsubstituted heterocycloalkyl), or -L⁹-(substituted or unsubstituted aryl);
      L⁹ is a bond, —O—, C(═O), —S—, —S(═O)—, —S(═O)₂—, —NH—, —NHC(═O)O—, —NHC(═O)NH—, —OC(═O)O—, —OC(═O)NH—, —NHC(═O)—, —C(═O)NH—, —C(═O)O—, or —OC(═O)—;
V is a bond, —C(═O)—, —C(OH)R¹³—, —(CR¹²R¹³)—, —NH—, —C(═O)NH—, —NHC(═O)—, —S—, —S(═O)—, or —S(═O)₂—;
R¹² is H, halogen, (substituted or unsubstituted C₁-C₆alkyl), (substituted or unsubstituted C₃-C₈cycloalkyl);
R¹³ is H, (substituted or unsubstituted C₁-C₆alkyl); or
R¹² and R¹³ taken together with the carbon to which they are attached may join to form a C₃-C₈cycloalkyl.

In further or alternative embodiments, provided herein are active metabolites, pharmaceutically acceptable solvates, pharmaceutically acceptable salts, pharmaceutically acceptable N-oxides, and pharmaceutically acceptable prodrugs of a compound of Formula (A).

In further or alternative embodiment, R¹¹ comprises at least one unsubstituted or substituted aromatic moiety (e.g., ring) and at least one unsubstituted or substituted heterocyclic moiety (e.g., ring). In certain embodiments, the unsubstituted or substituted heterocyclic moiety is an unsubstituted or substituted heterocycloalkyl moiety (e.g., ring) or an unsubstituted or substituted heteroaryl moiety (e.g., ring). In specific embodiments, R¹¹ is not a thienyl-phenyl group.

For any and all of the embodiments, substituents can be selected from among from a subset of the listed alternatives. For example, in some embodiments, Z is selected from among —[C(R¹)₂]ₘ—[C(R²)₂]ₙ—, —[C(R²)₂]ₙ—[C(R¹)₂]ₘ—O—, and —O—[C(R¹)₂]ₘ—[C(R²)₂]ₙ—; and Y is H, -(substituted or unsubstituted C₁-C₆alkyl), -(substituted or unsubstituted aryl), -(substituted or unsubstituted heteroaryl), or -(substituted or unsubstituted heterocycloalkyl). In further or alternative embodiments, Z is selected from among —C(R¹)₂—[C(R²)₂]ₙ—, —[C(R²)₂]ₙ—[C(R¹)₂]—O—, and —O—C(R¹)₂—[C(R²)₂]ₙ—. In further or alternative embodiments, each R¹ is independently H, —CF₃, or —CH₃. In further or alternative embodiments, Z is selected from among —CH₂—[C(R²)₂]ₙ—, —CH(Me)—[C(R²)₂]ₙ, —[C(R²)₂]ₙ—CH₂—O—, —[C(R²)₂]—CH(Me)—O—, —O—CH₂—[C(R²)₂]ₙ, and —O—CH(Me)—[C(R²)₂]ₙ—. In further or alternative embodiments, Z is selected from among —CH₂—, —CH(Me)—, —CH(Me)—O—, —O—CH₂—, and —O—CH(Me)—. In further or alternative embodiments, Z is —[C(R₂)₂]ₙC(R₁)₂O—.

In further or alternative embodiments, each R² is independently H, —CF₃, or an optionally substituted C₁-C₄alkyl.

In further or alternative embodiments, Y is H, -(substituted or unsubstituted C₁-C₆alkyl), -(substituted or unsubstituted aryl), -(substituted or unsubstituted heteroaryl), or -(substituted or unsubstituted heterocycloalkyl). Y is H, -(substituted or unsubstituted C₁-C₆alkyl), -(substituted or unsubstituted aryl), -(substituted or unsubstituted heteroaryl containing 0-1 S atoms, 0-1 O atoms, and 0-3 N atoms), or -(substituted or unsubstituted heterocycloalkyl containing 0-2 N atoms).

In further or alternative embodiments, Y is H, -(substituted or unsubstituted C₁-C₆alkyl), or -(substituted or unsubstituted aryl).

In further or alternative embodiments, Y is a -(substituted or unsubstituted heteroaryl), or -(substituted or unsubstituted heterocycloalkyl).

In further or alternative embodiments, Y is a -(substituted or unsubstituted heteroaryl). In specific embodiments, Y is a -(substituted or unsubstituted heteroaryl containing 0-1 S atoms, 0-1 O atoms, and 0-3 N atoms). In further or alternative embodiments, Y is a substituted or unsubstituted group selected from among pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, naphthyridinyl, imidazo[1,2-a]pyridinyl, thiophenopyridinyl, and furopyridinyl.

In further or alternative embodiments, Y is a substituted or unsubstituted group selected from among pyridinyl, pyrimidinyl, pyrazinyl, quinolinyl, isoquinolinyl, indolyl, benzimidazolyl, benzofuranyl, cinnolinyl, indazolyl, indolizinyl, phthalazinyl, pyridazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, naphthyridinyl, imidazo[1,2-a]pyridinyl, thiophenopyridinyl, and furopyridinyl. In some embodiments, Y is a substituted or unsubstituted group selected from among pyridinyl, pyrimidinyl, pyrazinyl, quinolinyl, isoquinolinyl, pyridazinyl, quinazolinyl, quinoxalinyl. In certain embodiments, Y is a substituted or unsubstituted group selected from among pyridinyl, and quinolinyl.

In further or alternative embodiments, Y is a -(substituted or unsubstituted heterocycloalkyl). In further or alternative embodiments, Y is a substituted or unsubstituted group selected from among quinolizinyl, dioxinyl, piperidinyl, morpholinyl, thiazinyl, tetrahydropyridinyl, piperazinyl, oxazinanonyl, dihydropyrrolyl, dihydroimidazolyl, tetrahydrofuranyl, dihydrooxazolyl, oxiranyl, pyrrolidinyl, pyrazolidinyl, dihydrothienyl, imidazolidinonyl, pyrrolidinonyl, dihydrofuranonyl, dioxolanonyl, thiazolidinyl, piperidinonyl, indolinyl, indanyl, tetrahydronaphthalenyl, tetrahydroquinolinyl, tetrahydrothienyl, and thiazepanyl.

In further or alternative embodiments, Y is selected from among:

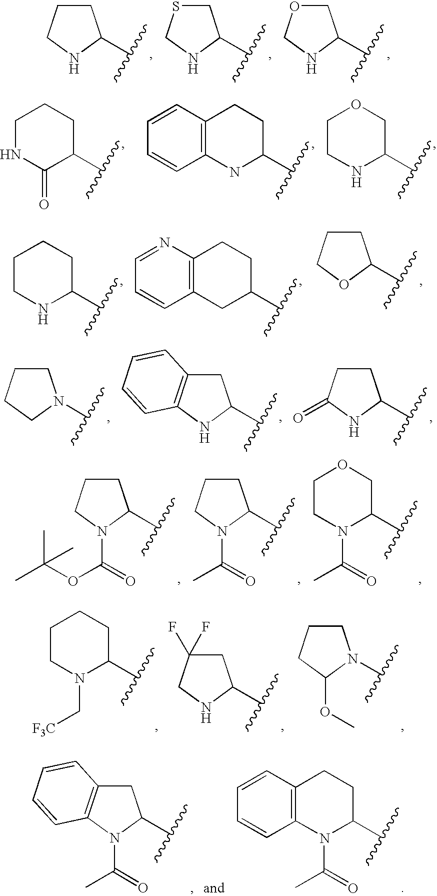

In further or alternative embodiments, R⁵ is H, halogen, —CH₃, or —OCH₃. In certain embodiments, R⁵ is H.

In further or alternative embodiments, R⁶ is H, -L²-(substituted or unsubstituted C₁-C₆alkyl), -L²-(substituted or unsubstituted C₃-C₈cycloalkyl), -L²-(substituted or unsubstituted heterocycloalkyl), -L²-(substituted or unsubstituted heteroaryl), or -L²-(substituted or unsubstituted aryl); and L² is a bond, —S(═O)₂—, —C(═O)—, or -(substituted or unsubstituted C₁-C₆alkyl). In certain embodiments, L² is a bond, —C(═O)—, or -(substituted or unsubstituted C₁-C₆alkyl)-.

In further or alternative embodiments, R⁶ is H, -L²-(substituted or unsubstituted C₁-C₆alkyl), -L²-(substituted or unsubstituted C₃-C₈cycloalkyl), or -L²-(substituted or unsubstituted aryl).

In further or alternative embodiments, V is a bond, —C(═O)—, —C(OH)R¹³—, —CR¹²R¹³—, —NH—, —C(═O)NH, —NHC(═O)—, —S—, —S(═O)—, or —S(═O)₂—; R¹² is H, halogen, or (substituted or unsubstituted C₁-C₆alkyl); R¹³ is H, (substituted or unsubstituted C₁-C₆alkyl). In further or alternative embodiments, V is a bond, —C(═O)—, —C(OH)H—, —CR¹²H—, —NH—, —C(═O)NH, —NHC(═O)—, —S—, —S(═O)—, or —S(═O)₂—. In further or alternative embodiments, V is a bond, —C(═O)—, —C(OH)H—, —CR¹²H—, —S—, —S(═O)—, or —S(═O)₂—. In further or alternative embodiments, V is —C(═O)—, —C(OH)H—, —CR¹²H—, —S—, —S(═O)—, or —S(═O)₂—.

In further or alternative embodiments, X is a bond, —O—, —C(═O)—, —CR⁹(OR⁹)—, —NR⁹—, —NR⁹C(═O)—, —C(═O)NR⁹—, aryl, heteroaryl or —NR⁹C(═O)NR⁹—.

In further or alternative embodiments, X is a bond, —O—, —C(═O)—, —CR⁹(OR⁹)—, —NR⁹—, —NR⁹C(═O)—, —C(═O)NR⁹—, or —NR⁹C(═O)NR⁹—.

In further or alternative embodiments, X is a bond, —O—, —C(═O)—, —CH(OH)—, —NH—, —NHC(═O)—, —C(═O)NH—, or —NHC(═O)NH—.

In further or alternative embodiments, X is a bond.

In further or alternative embodiments, G¹ is H, tetrazolyl, —OR⁹, —C(═O)NHS(═O)₂R⁸, —S(═O)₂NHC(═O)R⁸, —CN, —N(R⁹)₂, —N(R⁹)C(═O)R⁸, —NR⁹C(═CHR¹⁰)N(R⁹)₂, —CO₂R⁹, —C(═O)R⁹, —CON(R⁹)₂, —SR⁸, —S(═O)R⁸, —S(═O)₂R⁸, -L⁵-(substituted or unsubstituted C₁-C₆alkyl), -L⁵-(substituted or unsubstituted heteroaryl), or -L⁵-(substituted or unsubstituted aryl); and L⁵ is —NHC(═O)O, —NHC(═O), —C(═O)NH, —C(═O)O—, or —OC(═O)—.

In further or alternative embodiments, G¹ is H, tetrazolyl, —OR⁹, —CN, —N(R⁹)C(═O)R⁸, —CO₂R⁹, —C(═O)R⁹, —CON(R⁹)₂, —S(═O)R⁸, —S(═O)₂R⁸, -L⁵-(substituted or unsubstituted C₁-C₆alkyl), -L⁵-(substituted or unsubstituted heteroaryl), or -L⁵-(substituted or unsubstituted aryl).

In further or alternative embodiments, G¹ is H, tetrazolyl, —OR⁹, —CN, —N(R⁹)C(═O)R⁹, —CO₂R⁹, C(═O)R⁹, —CON(R⁹)₂, —SR⁸, —S(═O)₂R⁸, -L⁵-(substituted or unsubstituted C₁-C₆alkyl), -L⁵-(substituted or unsubstituted heteroaryl).

In so further or alternative me embodiments, G¹ is H, tetrazolyl, —OR⁹, —CN, —CO₂R⁹, —CON(R⁹)₂, -L⁵-(substituted or unsubstituted C₁-C₆alkyl), -L⁵-(substituted or unsubstituted heteroaryl).

In further or alternative embodiments, G¹ is H, tetrazolyl, —OR⁹, —CO₂R⁹, —CON(R⁹)₂, -L⁵-(substituted or unsubstituted C₁-C₆alkyl), -L⁵-(substituted or unsubstituted heteroaryl).

In further or alternative embodiments, G¹ is W-G².

In further or alternative embodiments, W is a (substituted or unsubstituted heterocycloalkyl), or a (substituted or unsubstituted heteroaryl); G² is H, tetrazolyl, —NHS(═O)₂R⁸, S(═O)₂N(R⁹)₂, OH, —OR^{B}, —C(═O)CF₃, CN, N(R⁹)₂, —N(R⁹)C(═O)R⁸, —C(═NR¹⁰)N(R⁹)₂, —CO₂R⁹, —C(═O)R⁹, —CON(R⁹)₂, —S(═O)R⁸, or —S(═O)₂R⁸.

In further or alternative embodiments, G² is H, tetrazolyl, —OH, —OR⁸, —C(═O)CF₃, CN, N(R⁹)₂, —N(R⁹)C(═O)R⁸, —CO₂R⁹, —C(═O)R⁹, —CON(R⁹)₂.

In further or alternative embodiments, each R⁸ is a (substituted or unsubstituted C₁-C₄alkyl); and each R⁹ is independently selected from among H, and (substituted or unsubstituted C₁-C₄alkyl).

In further or alternative embodiments, L⁷ is a bond; and L¹⁰ is a (substituted or unsubstituted heteroaryl), (substituted or unsubstituted aryl), or (substituted or unsubstituted heterocycloalkyl).

In further or alternative embodiments, G³ is tetrazolyl, -L⁸-(substituted or unsubstituted heteroaryl), or -L⁸-(substituted or unsubstituted aryl); and L⁸ is —O—, —C(═O)—, —C(═O)NH—, —C(═O)O—, or —OC(═O)—.

In further or alternative embodiments, G³ is W⁴-G⁴.

In further or alternative embodiments, W⁴ is (substituted or unsubstituted heterocycloalkyl), (substituted or unsubstituted aryl) or a (substituted or unsubstituted heteroaryl); and G⁴ is H, halogen, —CN, —CF₃, —OCF₃, C₁-C₆alkyl, C₃-C₈cycloalkyl, C₁-C₆fluoroalkyl, tetrazolyl, —OH, —OR⁸, —C(═O)CF₃, —CN, —CO₂R⁹, —C(═O)R⁹, —CON(R⁹)₂, -L⁹-(substituted or unsubstituted C₁-C₆alkyl), -L⁹-(substituted or unsubstituted C₁-C₆heteroalkyl), -L⁹-(substituted or unsubstituted heteroaryl), -L⁹-(substituted or unsubstituted heterocycloalkyl), or -L⁹-(substituted or unsubstituted aryl); and L⁹ is a bond, —O—, C(═O), —S—, —S(═O)—, —S(═O)₂—, —NH—, —NHC(═O)O—, —OC(═O)O—, —OC(═O)NH—, —NHC(═O)—, —C(═O)NH—, —C(═O)O—, or —OC(═O)—.

In further or alternative embodiments, L¹⁰ is (substituted or unsubstituted aryl); and G³ is W⁴-G⁴, where W⁴ is (substituted or unsubstituted heterocycloalkyl), or a (substituted or unsubstituted heteroaryl).

In further or alternative embodiments, provided herein are compounds of Formula (B), pharmaceutically acceptable salts, pharmaceutically acceptable N-oxides, pharmaceutically active metabolites, pharmaceutically acceptable prodrugs, and pharmaceutically acceptable solvates thereof. In some embodiments, compounds of Formula (B) antagonize or inhibit FLAP. In further or alternative embodiments, compounds of Formula (B) are used to treat patients suffering from leukotriene-dependent or leukotriene mediated conditions or diseases, including, but not limited to, asthma, myocardial infarction, cancer, and inflammatory conditions.

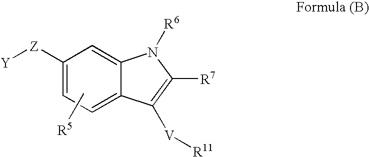

wherein:
Z is selected from —O—[C(R¹)₂]ₘ—[C(R²)₂]ₙ— and —[C(R²)₂]ₙ—[C(R¹)₂]ₘ—O—;
   each R¹ is independently H, —CF₃, or an optionally substituted C₁-C₄alkyl; or
   two R¹ on the same carbon taken together with the carbon to which they are attached form a carbonyl (C═O);
   each R² is independently H, —OH, —OMe, —CF₃, or an optionally substituted C₁-C₄alkyl; or
   two R² on the same carbon taken together with the carbon to which they are attached form a carbonyl (C═O);
   m is 0, 1 or 2;
   n is 0, 1, 2, or 3;
Y is -(substituted or unsubstituted aryl), -(substituted or unsubstituted heteroaryl); or (substituted or unsubstituted heterocycloalkyl);
wherein if Y is substituted, then each substitutent of Y is independently (L^{s}R^{s}),
   each L^{s} is independently selected from a bond and —C(═O)—;
   each R^{s} is halogen or C₁-C₆alkyl;
R⁵ is H, halogen, substituted or unsubstituted C₁-C₆alkyl, or substituted or unsubstituted —O—(C₁-C₆alkyl);
R⁶ is selected from C₁-C₆alkyl, C₃-C₈cycloalkyl, C₁-C₆alkyl-C₃-C₈cycloalkyl, aryl, and C₁-C₆alkyl-aryl; and
R⁷ is -L³-G¹;
   L³ is a C₁-C₆alkyl;
   G¹ is selected from H, tetrazolyl, —C(NH)N(R⁹)₂, —NR⁹C(NH)N(R⁹)₂, —NR⁹C(═CH₂)N(R⁹)₂, —C(═O)NR⁹C(═NH)N(R⁹)₂, —C(═O)NR⁹C(═CH₂)N(R⁹)₂, —CO₂R⁹, and —CON(R⁹)₂;
      each R⁹ is independently selected from H and C₁-C₄alkyl;
R¹¹ is —O—W⁴-G⁴, wherein
   L¹⁰ is a substituted or unsubstituted aryl;
   W⁴ is a -(substituted or unsubstituted heteroaryl) or a -(substituted or unsubstituted heterocycloalkyl); and
   G⁴ is H, halogen, C₁-C₆alkyl, C₁-C₆alkoxy, or —CF₃;
V is a bond, —(CR¹²R¹³)—, —S—, —S(═O)—, or —S(═O)₂—;
R¹² is H, halogen, or C₁-C₆alkyl;
R¹³ is H, or C₁-C₆alkyl; or
R¹² and R¹³ taken together with the carbon to which they are attached may join to form a C₃-C₈cycloalkyl.

In specific embodiments, R⁵ is H, L¹⁰ is phenyl, L³ is —CH₂C(CH₃)₂—. Accordingly, in some embodiments, the compounds presented herein have a structure of Formula (C):

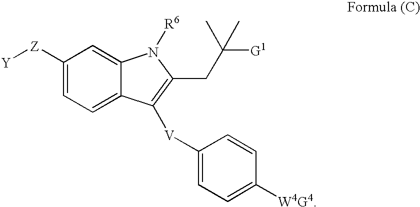

In further or alternative embodiments, Z, Y, R⁶, G¹, V, W⁴ and G⁴ are as set forth for Formula (A). In specific embodiments, Y, R⁶, G¹, V, W⁴ and G⁴ are as set forth for Formula (B). In specific embodiments, Z is —OCH₂— or —CH₂O—.

In further or alternative embodiments, W⁴ is selected from substituted and unsubstituted forms of any of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, naphthyridinyl, imidazo[1,2-a]pyridinyl, thiophenopyridinyl, and furopyridinyl. In specific embodiments, W⁴ is selected from substituted and unsubstituted forms of any of pyridinyl, pyrimidinyl and thiazolyl.

In further or alternative embodiments, Y is selected from -(substituted or unsubstituted heteroaryl) and (substituted or unsubstituted heterocycloalkyl). In further or alternative embodiments, Y is selected from optionally substituted pyridinyl, optionally substituted quinolinyl, optionally substituted pyrrolidinyl, optionally substituted indolyl, and optionally substituted 2,3-dihydro-1H-indolyl.

In a further or alternative embodiment, the “G” group (e.g. G′, G², G³, G⁴) of any of Formulas (A), (B) or (C) is any group that is used to tailor the physical and biological properties of the molecule. Such tailoring/modifications are achieved using groups which modulate acidity, basicity, lipophilicity, solubility and other physical properties of the molecule. The physical and biological properties modulated by such modifications to “G” include, by way of example only, solubility, in vivo absorption, and in vivo metabolism. In addition, in vivo metabolism may include, by way of example only, controlling in vivo PK properties, off-target activities, potential toxicities associated with cypP450 interactions, drug-drug interactions, and the like. Further, modifications to “G” allow for the tailoring of the in vivo efficacy of the compound through the modulation of, by way of example, specific and non-specific protein binding to plasma proteins and lipids and tissue distribution in vivo. Additionally, such tailoring/modifications to “G” allow for the design of compounds selective for 5-lipoxygenase-activating protein over other proteins.

In further or alternative embodiments, “G” is L²⁰-Q, wherein L²⁰ is an enzymatically cleavable linker and Q is a drug, or an affinity moiety. In further or alternative embodiments, the drug includes, by way of example only, leukotriene receptor antagonists and anti-inflammatory agents. In further or alternative embodiments, the leukotriene receptor antagonists include, but are not limited to, CysLT1/CysLT2 dual antagonists and CysLT1 antagonists. In further or alternative embodiments, the affinity moiety allows for site specific binding and include, but are not limited to, antibodies, antibody fragments, DNA, RNA, siRNA, and ligands.

Any combination of the groups described above for the various variables is contemplated herein.

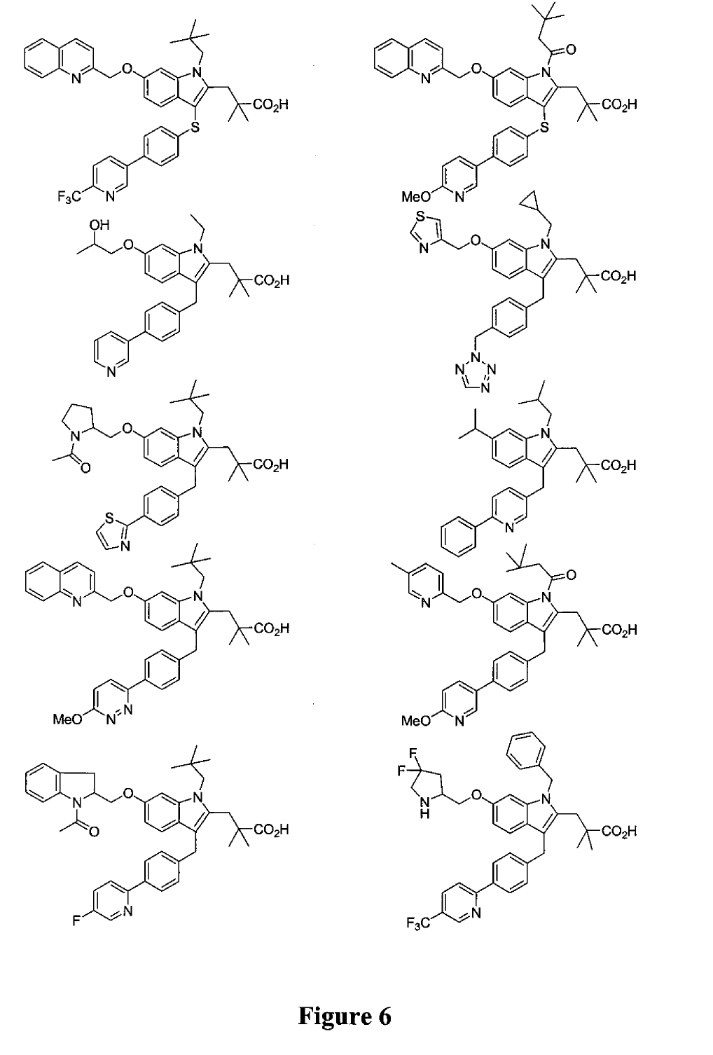

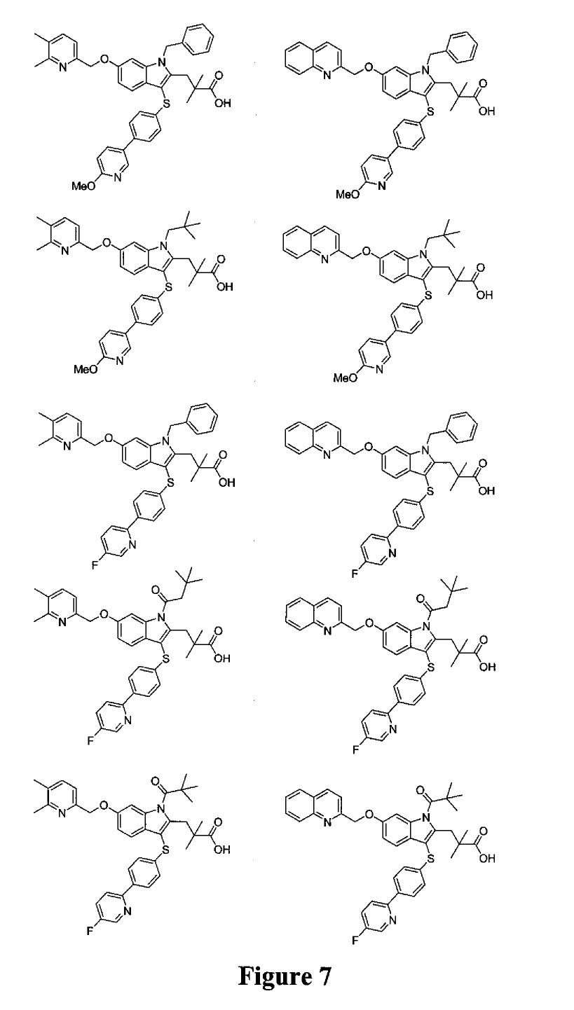

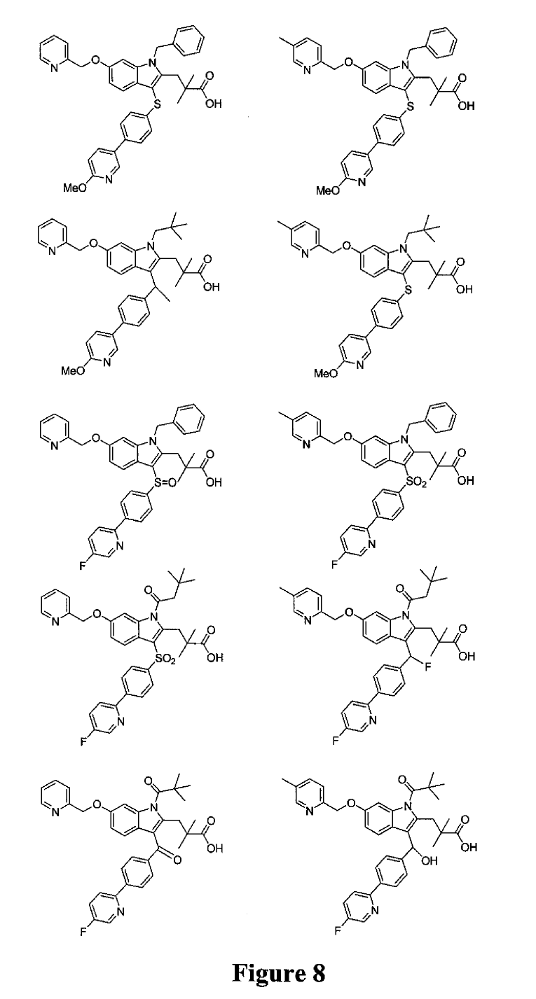

Specific embodiments of compounds of Formula (A) include, but are not limited to, compounds shown in FIGS. 6-8. In further or alternative embodiments, compounds of Formulas (A), (B), and (C) include compounds of Formula (C), wherein G¹ is —COOH, and ZY, R⁶, V and W⁴G⁴ are set forth in Table 1.

TABLE 1

| | ZY | R⁶ | V | W⁴G⁴ |
|---|---|---|---|---|
| 1 | Pyridin-2-ylmethoxy | Benzyl | S | 6′-Ethoxy-pyridin-3′-yl |
| 2 | Pyridin-2-ylmethoxy | Benzyl | S | 5′-Fluoro-pyridin-2′-yl |
| 3 | Pyridin-2-ylmethoxy | Benzyl | S | 5′-Methoxy-pyrimidin-2′-yl |
| 4 | Pyridin-2-ylmethoxy | Benzyl | S | 5′-Trifluoromethyl-pyridin-2′-yl |
| 5 | Pyridin-2-ylmethoxy | Benzyl | S | 6′-Trifluoromethyl-pyridin-3′-yl |
| 6 | Pyridin-2-ylmethoxy | Benzyl | S | 2′-Ethoxy-thiazol-4′-yl |
| 7 | Pyridin-2-ylmethoxy | 3-methylbutyl | S | 6′-Ethoxy-pyridin-3′-yl |
| 8 | Pyridin-2-ylmethoxy | 3-methylbutyl | S | 5′-Fluoro-pyridin-2′-yl |
| 9 | Pyridin-2-ylmethoxy | 3-methylbutyl | S | 5′-Methoxy-pyrimidin-2′-yl |
| 10 | Pyridin-2-ylmethoxy | 3-methylbutyl | S | 5′-Trifluoromethyl-pyridin-2′-yl |
| 11 | Pyridin-2-ylmethoxy | 3-methylbutyl | S | 6′-Trifluoromethyl-pyridin-3′-yl |
| 12 | Pyridin-2-ylmethoxy | 3-methylbutyl | S | 2′-Ethoxy-thiazol-4′-yl |
| 13 | 5-Methyl-pyridin-2-ylmethoxy | Benzyl | S | 6′-Ethoxy-pyridin-3′-yl |
| 14 | 5-Methyl-pyridin-2-ylmethoxy | Benzyl | S | 5′-Fluoro-pyridin-2′-yl |
| 15 | 5-Methyl-pyridin-2-ylmethoxy | Benzyl | S | 5′-Methoxy-pyrimidin-2′-yl |
| 16 | 5-Methyl-pyridin-2-ylmethoxy | Benzyl | S | 5′-Trifluoromethyl-pyridin-2′-yl |
| 17 | 5-Methyl-pyridin-2-ylmethoxy | Benzyl | S | 6′-Trifluoromethyl-pyridin-3′-yl |
| 18 | 5-Methyl-pyridin-2-ylmethoxy | Benzyl | S | 2′-Ethoxy-thiazol-4′-yl |
| 19 | 5-Methyl-pyridin-2-ylmethoxy | 3-methylbutyl | S | 6′-Ethoxy-pyridin-3′-yl |
| 20 | 5-Methyl-pyridin-2-ylmethoxy | 3-methylbutyl | S | 5′-Fluoro-pyridin-2′-yl |
| 21 | 5-Methyl-pyridin-2-ylmethoxy | 3-methylbutyl | S | 5′-Methoxy-pyrimidin-2′-yl |
| 22 | 5-Methyl-pyridin-2-ylmethoxy | 3-methylbutyl | S | 5′-Trifluoromethyl-pyridin-2′-yl |
| 23 | 5-Methyl-pyridin-2-ylmethoxy | 3-methylbutyl | S | 6′-Trifluoromethyl-pyridin-3′-yl |
| 24 | 5-Methyl-pyridin-2-ylmethoxy | 3-methylbutyl | S | 2′-Ethoxy-thiazol-4′-yl |
| 25 | Quinolin-2-ylmethoxy | Benzyl | S | 6′-Ethoxy-pyridin-3′-yl |
| 26 | Quinolin-2-ylmethoxy | Benzyl | S | 5′-Fluoro-pyridin-2′-yl |
| 27 | Quinolin-2-ylmethoxy | Benzyl | S | 5′-Methoxy-pyrimidin-2′-yl |
| 28 | Quinolin-2-ylmethoxy | Benzyl | S | 5′-Trifluoromethyl-pyridin-2′-yl |
| 29 | Quinolin-2-ylmethoxy | Benzyl | S | 6′-Trifluoromethyl-pyridin-3′-yl |
| 30 | Quinolin-2-ylmethoxy | Benzyl | S | 2′-Ethoxy-thiazol-4′-yl |
| 31 | Quinolin-2-ylmethoxy | 3-methylbutyl | S | 6′-Ethoxy-pyridin-3′-yl |
| 32 | Quinolin-2-ylmethoxy | 3-methylbutyl | S | 5′-Fluoro-pyridin-2′-yl |
| 33 | Quinolin-2-ylmethoxy | 3-methylbutyl | S | 5′-Methoxy-pyrimidin-2′-yl |
| 34 | Quinolin-2-ylmethoxy | 3-methylbutyl | S | 5′-Trifluoromethyl-pyridin-2′-yl |
| 35 | Quinolin-2-ylmethoxy | 3-methylbutyl | S | 6′-Trifluoromethyl-pyridin-3′-yl |
| 36 | Quinolin-2-ylmethoxy | 3-methylbutyl | S | 2′-Ethoxy-thiazol-4′-yl |
| 37 | (S)-1-Acetyl-pyrrolidin-2- | Benzyl | S | 6′-Ethoxy-pyridin-3′-yl |
| | ylmethoxy | | | |
| 38 | (S)-1-Acetyl-pyrrolidin-2- | Benzyl | S | 5′-Fluoro-pyridin-2′-yl |
| | ylmethoxy | | | |
| 39 | (S)-1-Acetyl-pyrrolidin-2- | Benzyl | S | 5′-Methoxy-pyrimidin-2′-yl |
| | ylmethoxy | | | |
| 40 | (S)-1-Acetyl-pyrrolidin-2- | Benzyl | S | 5′-Trifluoromethyl-pyridin-2′-yl |
| | ylmethoxy | | | |
| 41 | (S)-1-Acetyl-pyrrolidin-2- | Benzyl | S | 6′-Trifluoromethyl-pyridin-3′-yl |
| | ylmethoxy | | | |
| 42 | (S)-1-Acetyl-pyrrolidin-2- | Benzyl | S | 2′Ethoxy-thiazol-4′-yl |
| | ylmethoxy | | | |
| 43 | (S)-1-Acetyl-pyrrolidin-2- | 3-methylbutyl | S | 6′-Ethoxy-pyridin-3′-yl |
| | ylmethoxy | | | |
| 44 | (S)-1-Acetyl-pyrrolidin-2- | 3-methylbutyl | S | 5′-Fluoro-pyridin-2′-yl |
| | ylmethoxy | | | |
| 45 | (S)-1-Acetyl-pyrrolidin-2- | 3-methylbutyl | S | 5′-Methoxy-pyrimidin-2′-yl |
| | ylmethoxy | | | |
| 46 | (S)-1-Acetyl-pyrrolidin-2- | 3-methylbutyl | S | 5′-Trifluoromethyl-pyridin-2′-yl |
| | ylmethoxy | | | |
| 47 | (S)-1-Acetyl-pyrrolidin-2- | 3-methylbutyl | S | 6′-Trifluoromethyl-pyridin-3′-yl |
| | ylmethoxy | | | |
| 48 | (S)-1-Acetyl-pyrrolidin-2- | 3-methylbutyl | S | 2′-Ethoxy-thiazol-4′-yl |
| | ylmethoxy | | | |
| 49 | (S)-1-Acetyl-2,3-dihydro-1H- | Benzyl | S | 6′-Ethoxy-pyridin-3′-yl |
| | indol-2-ylmethoxy | | | |
| 50 | (S)-1-Acetyl-2,3-dihydro-1H- | Benzyl | S | 5′-Fluoro-pyridin-2′-yl |
| | indol-2-ylmethoxy | | | |
| 51 | (S)-1-Acetyl-2,3-dihydro-1H- | Benzyl | S | 5′-Methoxy-pyrimidin-2′-yl |
| | indol-2-ylmethoxy | | | |
| 52 | (S)-1-Acetyl-2,3-dihydro-1H- | Benzyl | S | 5′-Trifluoromethyl-pyridin-2′-yl |
| | indol-2-ylmethoxy | | | |
| 53 | (S)-1-Acetyl-2,3-dihydro-1H- | Benzyl | S | 6′-Trifluoromethyl-pyridin-3′-yl |
| | indol-2-ylmethoxy | | | |
| 54 | (S)-1-Acetyl-2,3-dihydro-1H- | Benzyl | S | 2′-Ethoxy-thiazol-4′-yl |
| | indol-2-ylmethoxy | | | |
| 55 | (S)-1-Acetyl-2,3-dihydro-1H- | 3-methylbutyl | S | 6′-Ethoxy-pyridin-3′-yl |
| | indol-2-ylmethoxy | | | |
| 56 | (S)-1-Acetyl-2,3-dihydro-1H- | 3-methylbutyl | S | 5′-Fluoro-pyridin-2′-yl |
| | indol-2-ylmethoxy | | | |
| 57 | (S)-1-Acetyl-2,3-dihydro-1H- | 3-methylbutyl | S | 5′-Methoxy-pyrimidin-2′-yl |
| | indol-2-ylmethoxy | | | |
| 58 | (S)-1-Acetyl-2,3-dihydro-1H- | 3-methylbutyl | S | 5′-Trifluoromethyl-pyridin-2′-yl |
| | indol-2-ylmethoxy | | | |
| 59 | (S)-1-Acetyl-2,3-dihydro-1H- | 3-methylbutyl | S | 6′-Trifluoromethyl-pyridin-3′-yl |
| | indol-2-ylmethoxy | | | |
| 60 | (S)-1-Acetyl-2,3-dihydro-1H- | 3-methylbutyl | S | 2′-Ethoxy-thiazol-4′-yl |
| | indol-2-ylmethoxy | | | |
| 61 | 5-Methyl-pyridin-2-ylmethoxy | Benzyl | CH₂ | 6′-Ethoxy-pyridin-3′-yl |
| 62 | 5-Methyl-pyridin-2-ylmethoxy | Benzyl | CH₂ | 5′-Fluoro-pyridin-2′-yl |
| 63 | 5-Methyl-pyridin-2-ylmethoxy | Benzyl | CH₂ | 5′-Methoxy-pyrimidin-2′-yl |
| 64 | 5-Methyl-pyridin-2-ylmethoxy | Benzyl | CH₂ | 5′-Trifluoromethyl-pyridin-2′-yl |
| 65 | 5-Methyl-pyridin-2-ylmethoxy | Benzyl | CH₂ | 6′-Trifluoromethyl-pyridin-3′-yl |
| 66 | 5-Methyl-pyridin-2-ylmethoxy | Benzyl | CH₂ | 2′Ethoxy-thiazol-4′-yl |
| 67 | 5-Methyl-pyridin-2-ylmethoxy | 3-methylbutyl | CH₂ | 6′-Ethoxy-pyridin-3′-yl |
| 68 | 5-Methyl-pyridin-2-ylmethoxy | 3-methylbutyl | CH₂ | 5′-Fluoro-pyridin-2′-yl |
| 69 | 5-Methyl-pyridin-2-ylmethoxy | 3-methylbutyl | CH₂ | 5′-Methoxy-pyrimidin-2′-yl |
| 70 | 5-Methyl-pyridin-2-ylmethoxy | 3-methylbutyl | CH₂ | 5′-Trifluoromethyl-pyridin-2′-yl |
| 71 | 5-Methyl-pyridin-2-ylmethoxy | 3-methylbutyl | CH₂ | 6′-Trifluoromethyl-pyridin-3′-yl |
| 72 | 5-Methyl-pyridin-2-ylmethoxy | 3-methylbutyl | CH₂ | 2′-Ethoxy-thiazol-4′-yl |
| 73 | 5-Methyl-pyridin-2-ylmethoxy | Benzyl | S(O) | 6′-Ethoxy-pyridin-3′-yl |
| 74 | 5-Methyl-pyridin-2-ylmethoxy | Benzyl | S(O) | 5′-Fluoro-pyridin-2′-yl |
| 75 | 5-Methyl-pyridin-2-ylmethoxy | Benzyl | S(O) | 5′-Methoxy-pyrimidin-2′-yl |
| 76 | 5-Methyl-pyridin-2-ylmethoxy | Benzyl | S(O) | 5′-Trifluoromethyl-pyridin-2′-yl |
| 77 | 5-Methyl-pyridin-2-ylmethoxy | Benzyl | S(O) | 6′-Trifluoromethyl-pyridin-3′-yl |
| 78 | 5-Methyl-pyridin-2-ylmethoxy | Benzyl | S(O) | 2′-Ethoxy-thiazol-4′-yl |
| 79 | 5-Methyl-pyridin-2-ylmethoxy | 3-methylbutyl | S(O) | 6′-Ethoxy-pyridin-3′-yl |
| 80 | 5-Methyl-pyridin-2-ylmethoxy | 3-methylbutyl | S(O) | 5′-Fluoro-pyridin-2′-yl |
| 81 | 5-Methyl-pyridin-2-ylmethoxy | 3-methylbutyl | S(O) | 5′-Methoxy-pyrimidin-2′-yl |
| 82 | 5-Methyl-pyridin-2-ylmethoxy | 3-methylbutyl | S(O) | 5′-Trifluoromethyl-pyridin-2′-yl |
| 83 | 5-Methyl-pyridin-2-ylmethoxy | 3-methylbutyl | S(O) | 6′-Trifluoromethyl-pyridin-3′-yl |
| 84 | 5-Methyl-pyridin-2-ylmethoxy | 3-methylbutyl | S(O) | 2′-Ethoxy-thiazol-4′-yl |
| 85 | 5-Methyl-pyridin-2-ylmethoxy | Benzyl | S(O)₂ | 6′-Ethoxy-pyridin-3′-yl |
| 86 | 5-Methyl-pyridin-2-ylmethoxy | Benzyl | S(O)₂ | 5′-Fluoro-pyridin-2′-yl |
| 87 | 5-Methyl-pyridin-2-ylmethoxy | Benzyl | S(O)₂ | 5′-Methoxy-pyrimidin-2′-yl |
| 88 | 5-Methyl-pyridin-2-ylmethoxy | Benzyl | S(O)₂ | 5′-Trifluoromethyl-pyridin-2′-yl |
| 89 | 5-Methyl-pyridin-2-ylmethoxy | Benzyl | S(O)₂ | 6′-Trifluoromethyl-pyridin-3′-yl |
| 90 | 5-Methyl-pyridin-2-ylmethoxy | Benzyl | S(O)₂ | 2′-Ethoxy-thiazol-4′-yl |
| 91 | 5-Methyl-pyridin-2-ylmethoxy | 3-methylbutyl | S(O)₂ | 6′-Ethoxy-pyridin-3′-yl |
| 92 | 5-Methyl-pyridin-2-ylmethoxy | 3-methylbutyl | S(O)₂ | 5′-Fluoro-pyridin-2′-yl |
| 93 | 5-Methyl-pyridin-2-ylmethoxy | 3-methylbutyl | S(O)₂ | 5′-Methoxy-pyrimidin-2′-yl |
| 94 | 5-Methyl-pyridin-2-ylmethoxy | 3-methylbutyl | S(O)₂ | 5′-Trifluoromethyl-pyridin-2′-yl |
| 95 | 5-Methyl-pyridin-2-ylmethoxy | 3-methylbutyl | S(O)₂ | 6′-Trifluoromethyl-pyridin-3′-yl |
| 96 | 5-Methyl-pyridin-2-ylmethoxy | 3-methylbutyl | S(O)₂ | 2′-Ethoxy-thiazol-4′-yl |

Throughout the specification, groups and substituents thereof are chosen, in various embodiments, to provide stable moieties and compounds.

### Further Forms of Compounds

In certain embodiments, the compounds described herein possess one or more stereocenters. In various embodiments, each stereocenter is selected from the R and S configurations. The compounds presented herein include all diastereomeric, enantiomeric, and epimeric forms as well as the appropriate mixtures thereof. Separation of steroisomers is performed by any means. In some embodiments, stereoisomers separation is achieved, e.g., by chromatography. In alternative embodiments, individual stereoisomers are obtained by reacting a racemic mixture of the compound with an optically active resolving agent to form a pair of diastereoisomeric compounds, separating the diastereomers and recovering the optically pure enantiomers. While, in some embodiments, resolution of enantiomers is carried out using covalent diastereomeric derivatives of the compounds described herein, dissociable complexes are also contemplated herein (e.g., crystalline diastereomeric salts). Diastereomers have distinct physical properties (e.g., melting points, boiling points, solubilities, reactivity, etc.) and are, in some instances, readily separated by taking advantage of these dissimilarities. In various embodiments, the diastereomers are separated by chiral chromatography, or by separation/resolution techniques based upon differences in solubility. The optically pure enantiomer is then recovered, along with the resolving agent, by any practical means that would not result in racemization. Jean Jacques, Andre Collet, Samuel H. Wilen, “Enantiomers, Racemates and Resolutions”, John Wiley And Sons, Inc., 1981, are hereby incorporated by reference for such disclosure. In certain embodiments, stereoisomers are obtained by stereoselective synthesis.

In some situations, compounds described herein exist as tautomers. All tautomers are included within the formulas described herein.

The methods and formulations described herein include the use of N-oxides, crystalline forms (also known as polymorphs), or pharmaceutically acceptable salts of compounds described herein, as well as active metabolites of these compounds having the same type of activity. In some situations, compounds exist as tautomers. All tautomers are included within the scope of the compounds presented herein. In various embodiments, the compounds described herein exist in unsolvated as well as solvated forms with pharmaceutically acceptable solvents such as water, ethanol, and the like. The solvated forms of the compounds presented herein are also considered to be disclosed herein.

Compounds of Formulas (A), (B) and (C) in unoxidized form can be prepared from N-oxides of compounds of Formula (A), (B) and (C), by treating with a reducing agent, such as, but not limited to, sulfur, sulfur dioxide, triphenyl phosphine, lithium borohydride, sodium borohydride, phosphorus trichloride, phosphorus tribromide, or the like in a suitable inert organic solvent, such as, but not limited to, acetonitrile, ethanol, aqueous dioxane, or the like at 0 to 80° C.

In some embodiments, compounds described herein are prepared as prodrugs. A “prodrug” refers to an agent that is converted into the parent drug in vivo. In certain instances, prodrugs are often useful because, in some situations, they are easier to administer than the parent drug. For instance, in some cases, prodrugs are bioavailable when administered orally whereas the parent is not. In some embodiments, the prodrug has improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

To produce a prodrug, a pharmaceutically active compound is modified such that the active compound will be regenerated upon in vivo administration. In certain embodiments, a prodrug is designed to alter the metabolic stability or the transport characteristics of a drug, to mask side effects or toxicity, to improve the flavor of a drug, or to alter other characteristics or properties of a drug. In some embodiments, by virtue of knowledge of pharmacodynamic processes and drug metabolism in vivo, prodrugs of a compound are prepared and contemplated herein. Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401; and Saulnier et al., (1994), Bioorganic and Medicinal Chemistry Letters, Vol. 4, p. 1985 are hereby incorporated by reference for such disclosure.

Prodrug forms of the herein described compounds, wherein the prodrug is metabolized in vivo to produce a derivative as set forth herein are included within the scope of the claims. In some cases, some of the herein-described compounds are prodrugs for another derivative or active compound.

Prodrugs are often useful because, in some situations, they are easier to administer than the parent drug. For instance, in some embodiments, prodrugs are bioavailable by oral administration whereas the parent is not. In certain embodiments, the prodrug has improved solubility in pharmaceutical compositions over the parent drug. In some embodiments, prodrugs are designed as reversible drug derivatives, for use as modifiers to enhance drug transport to site-specific tissues. In some embodiments, the design of a prodrug increases the effective water solubility. Fedorak et al., Am. J. Physiol., 269:G210-218 (1995); McLoed et al., Gastroenterol, 106:405-413 (1994); Hochhaus et al., Blamed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, are all hereby incorporated by reference for such disclosure.

In certain embodiments, sites on the aromatic ring portion of compounds described herein are susceptible to various metabolic reactions, therefore incorporation of appropriate substituents on the aromatic ring structures minimize or eliminate this metabolic pathway. In some embodiments, appropriate substituents for minimizing or elimination metabolic decomposition of an aromatic ring includes substituents selected from, by way of non-limiting example, halogens.

In some embodiments, the compounds described herein are labeled isotopically (e.g. with a radioisotope) or by other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. In some embodiments, substitution with isotopes such as deuterium, i.e., ²H, afford certain therapeutic advantages resulting from greater metabolic stability, for example increased in vivo half-life or reduced dosage requirements.

In additional or further embodiments, the compounds described herein are metabolized upon administration to an organism in need to produce a metabolite that is then used to produce a desired effect, including a desired therapeutic effect.

Included in the compounds and methods used herein are the pharmaceutical acceptable salts of the compounds described herein. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, nitric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Acceptable organic bases include ethanolamine, diethanolamine, triethanolamine, tromethamine, N-methylglucamine, dicyclohexylamine, tris(hydroxymethyl)methylamine, and salts with amino acids such as arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent. In some embodiments, solvates are formed during the process of crystallization with pharmaceutically acceptable solvents such as water, ethanol, and the like. Hydrates are formed when the solvent is water; alcoholates are formed when the solvent is alcohol. In some embodiments, solvates of compounds described herein are conveniently prepared or formed during the processes described herein. In some embodiments, the compounds provided herein are in unsolvated or solvated form. In general, disclosure of any compound herein, as either a compound per se, in a composition or in a method, includes disclosure of the solvated as well as unsolvated form.

Compounds within the scope of the embodiments described herein are in any form, including but not limited to, amorphous forms, milled forms and nano-particulate forms. In addition, compounds described herein include crystalline forms, also known as polymorphs. Polymorphs include the different crystal packing arrangements of the same elemental composition of a compound. In certain embodiments, polymorphs have different X-ray diffraction patterns, infrared spectra, melting points, density, hardness, crystal shape, optical and electrical properties, stability, and solubility. In certain embodiments, various factors such as the recrystallization solvent, rate of crystallization, and storage temperature cause a single crystal form to dominate.

When desired, the screening and characterization of the pharmaceutically acceptable salts, polymorphs and/or solvates are accomplished using any technique. In various embodiments, techniques include, but are not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning Calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UV-VIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

Throughout the specification, groups and substituents thereof are chosen, in various embodiments, to provide stable moieties and compounds.

### Synthesis of Compounds

In various embodiments, the synthesis of compounds described herein are accomplished using any process. In some embodiments, such processes are described in the chemical literature, using the methods described herein, or by a combination thereof. In certain embodiments, the solvents, temperatures and other reaction conditions presented herein are varied in order to afford the desired product.

The starting materials used for the synthesis of the compounds described herein are from any source. In some embodiments, the starting materials are synthesized or obtained from commercial sources, such as, but not limited to, Aldrich Chemical Co. (Milwaukee, Wis.), or Sigma Chemical Co. (St. Louis, Mo.). The compounds described herein, and other related compounds having different substituents are synthesized using any techniques and materials, including, by way of non-limiting example, those described herein. March, ADVANCED ORGANIC CHEMISTRY 4^{th} Ed., (Wiley 1992); Carey and Sundberg, ADVANCED ORGANIC CHEMISTRY 4⁶ Ed., Vols. A and B (Plenum 2000, 2001), and Green and Wuts, PROTECTIVE GROUPS IN ORGANIC SYNTHESIS 3rd Ed., (Wiley 1999) are hereby incorporated by reference for such disclosure.

Formation of Covalent Linkages by Reaction of an Electrophile with a Nucleophile

In certain embodiments, the compounds described herein are modified using various electrophiles and/or nucleophiles to form new functional groups or substituents. Table 2 entitled “Examples of Covalent Linkages and Precursors Thereof” lists selected non-limiting examples of covalent linkages and precursor functional groups which yield the covalent linkages. In some embodiments, Table 2 is used as guidance toward the variety of electrophiles and nucleophiles combinations available that provide covalent linkages. Precursor functional groups are shown as electrophilic groups and nucleophilic groups.

TABLE 2
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

Use of Protecting Groups

In some embodiments of the reactions described, it is necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. In specific embodiments wherein more than one protecting group is utilized, each protecting group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. In some embodiments, acid labile groups such as trityl, dimethoxytrityl, acetal and t-butyldimethylsilyl are used to protect carboxy and hydroxy reactive moieties in the presence of amino groups protected with Cbz groups, which are removable by hydrogenolysis, and Fmoc groups, which are base labile. In certain embodiments, carboxylic acid and hydroxy reactive moieties are blocked with base labile groups such as, but not limited to, methyl, ethyl, and acetyl in the presence of amines blocked with acid labile groups such as t-butyl carbamate or with carbamates that are both acid and base stable but hydrolytically removable.

In some embodiments, carboxylic acid and hydroxy reactive moieties are blocked with hydrolytically removable protective groups such as the benzyl group, while amine groups capable of hydrogen bonding with acids are blocked, e.g., with base labile groups such as Fmoc. In certain embodiments, carboxylic acid reactive moieties are protected by conversion to simple ester compounds as exemplified herein, or they are blocked with oxidatively-removable protective groups such as 2,4-dimethoxybenzyl, while co-existing amino groups are blocked, e.g., with fluoride labile silyl carbamates.

In some embodiments, allyl blocking groups are useful in then presence of acid- and base-protecting groups since the former are stable and can be subsequently removed by metal or pi-acid catalysts. For example, in some embodiments, an allyl-blocked carboxylic acid is deprotected with a Pd⁰-catalyzed reaction in the presence of acid labile t-butyl carbamate or base-labile acetate amine protecting groups. In some embodiments, the protecting group is a resin to which a compound or intermediate is attached. As long as the residue is attached to the resin, that functional group is blocked and cannot react. Once released from the resin, the functional group is available to react.

In certain embodiments, blocking/protecting groups are selected from:

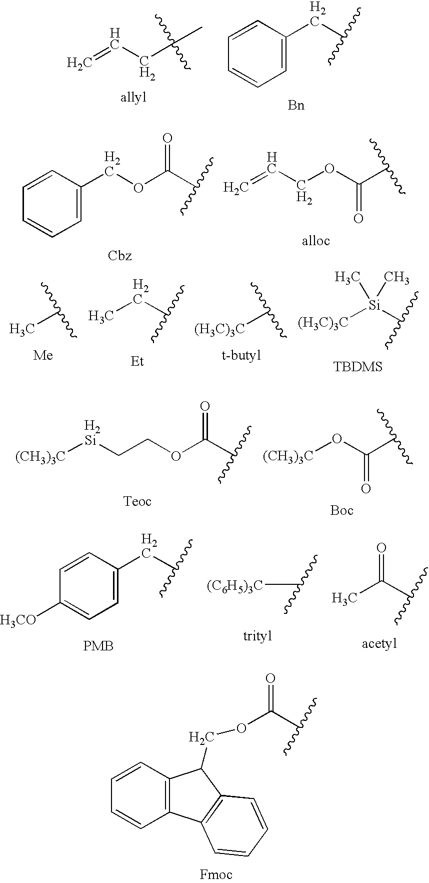

Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, are hereby incorporated by reference for such disclosure.

Indole containing compounds are prepared using any suitable process. In some embodiments, processes such as those set forth in Katritzky, “Handbook of Heterocyclic Chemistry” Pergamon Press, Oxford, 1986; Pindur et al, J. Heterocyclic Chem., vol 25, 1, 1987, and Robinson “The Fisher Indole Synthesis”, John Wiley & Sons, Chichester, N.Y., 1982, each of which is herein incorporated by reference for such disclosure, are utilized.

A non-limiting example of a synthetic approach toward indole compounds of Formulas (A), (B) and (C) is outlined in Scheme I, which begins with 3-substituted anilines of structure I-1.

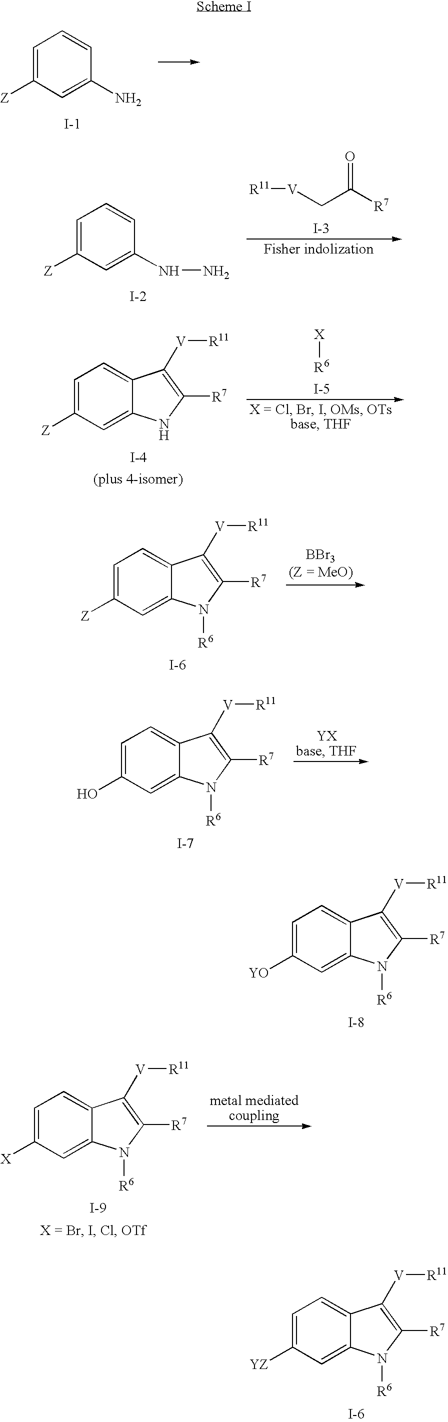

In certain embodiments, 3-Substituted anilines of structure I-1 are converted to the corresponding hydrazines of structure I-2 using standard methodology. In some embodiments, reaction of 3-substituted hydrazines of structure I-2 with an appropriately substituted ketone of structure I-3 under standard Fisher-indolization conditions yields indoles of structure I-4. In certain embodiments, indoles of structure I-6 are obtained from the N-alkylation of indoles of structure I-4 with an alkyl halide of structure I-5 (or benzyl halide, or tosylate (OTs) or mesylate (OMs), or carboxylic acid halide) in a solvent such as tetrahydrofuran (THF) or dimethylformamide (DMF) in the presense of a base, such as, for example, NaH. In certain embodiments wherein the 6-substituent on the indole ring is methoxy (i.e. Z is MeO), the methyl group is removed under standard conditions, for example using BBr₃, in a solvent such as CH₂Cl₂ to afford phenols of structure I-7. A preferred method for deprotection of the methoxy group is to use either pyridine hydrochloride at 175° C. or tBuSH/AlCl₃ in a solvent such as CH₂Cl₂. In some embodiments, phenols of structure I-7 are alkylated using an electrophile (YX) to provide the alkylated indoles of structure I-8. In certain embodiments, wherein the 6-substituent on the indole ring is, for example, a halide or triflate (OTf), such as, for example, indoles of structure I-9, it is coupled with a wide variety of reagents using standard metal mediated coupling reactions to afford alternate compounds of structure I-6. Comprehensive Organometallic Chemistry II, vol 12, Pergamon, edited by Abel, Stone and Wilkinson is hereby incorporated by reference for such disclosure. In certain embodiments, the Z substitutent of indoles of structure I-6 can be further modified using standard chemical procedures. In additional embodiments, wherein R⁷ or V—R¹¹ is a bromo or iodine, standard cross coupling reactions allow the introduction of a variety of functional groups. In some embodiments wherein R⁷ is H, the compound is regioselectively lithiated using a strong base such as nBuLi and then the anion is condensed with an electrophile to introduce substituents at C-2 of the indole. Hasan et al, J. Org. Chem., 46, 157-164 (1981) is hereby incorporated by reference for such disclosure.

Metal mediated coupling reactions include, but are not limited to, Suzuki reactions, Sonogashira couplings, Heck reactions, Stille cross couplings, Negishi couplings, Kumada couplings, Ullmann reactions, Buchwald-Hartwig reactions, and variants thereof. Metal-Catalyzed Cross-Coupling Reactions, Armin de Meijere (Editor), Francois Diederich (Editor), John Wiley & Sons; 2nd edition (2004) is hereby incorporated by reference for such disclosure.

Another non-limiting example of a synthetic approach toward compounds of Formula (A), (B) and (C) is shown in Scheme II.

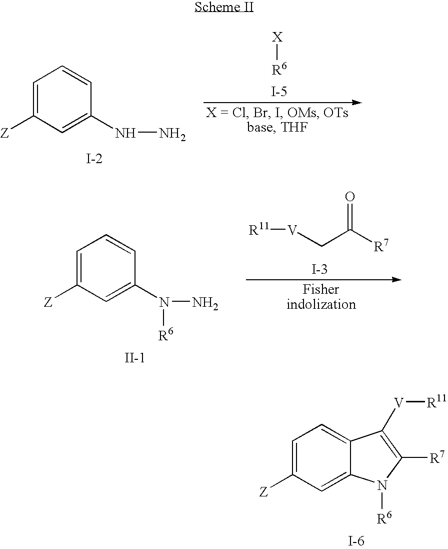

In certain embodiments, the synthetic process comprises presenting hydrazines of structure I-2, N-alkylating with a alkyl halide (or benzyl halide, or tosylate or mesylate; I-5) using, e.g., the conditions described above, and providing hydrazines of structure II-1. In some embodiments, reaction with an appropriately substituted ketone of structure I-3 using Fisher indolization conditions provides indoles of structure I-6.

Another non-limiting example of the synthetic approach toward compounds of Formula (A) is shown in Scheme III, wherein 3-H-indoles of structure III-1 are prepared by any process, including, the procedures described above or, alternatively, by treating 3-thioindoles with moist AlCl₃ in a solvent such as CH₂Cl₂.

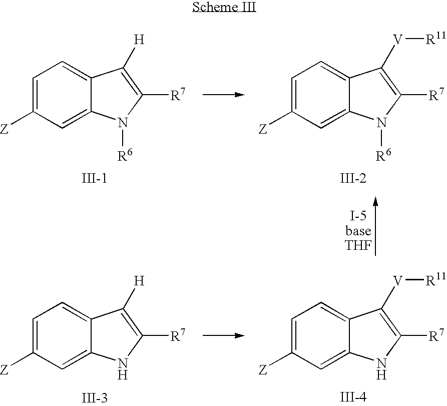

In certain embodiments, functionalization at the 3-position of 3-H-indoles of structure III-1 is achieved by using a variety of reactions and procedures to allow the introduction of a wide range of substituents. By way of example only, acylation using an acid chloride (or anhydride) in the presence of a Lewis acid such as AlCl₃, allows for the introduction of acyl groups (III-2; VR¹¹═C(O)R¹¹). Murakami et al. Heterocycles, v14, 1939-1941, 1980 and references cited therein are hereby incorporated by reference for such disclosure. In certain embodiments, commencing with 3-H-indoles of structure III-1, and using, by way of example only, sulfenic chlorides in a suitable solvent, compounds of general structure III-2 wherein VR¹¹ are SR¹¹ are prepared. Raban, J. Org. Chem., v45, 1688 (1980) is hereby incorporated by reference for such disclosure. In another approach, indoles of structure III-3 are reacted with diarlydisulfides or dialkyldisulfides in the presence of a base, such as NaH, in DMF to generate indoles of structure III-4. Atkinson et al, Synthesis, 480-481 (1988) is hereby incorporated by reference for such disclosure. In some embodiments, the reaction of electron deficient olefins with 3-H indoles of structure III-1 or structure III-3 in the presence of a Lewis acid (such as, for example, Yb(OTf)₃.3H₂O) allows the installation of alkyl substituents at the 3-position of the indole compounds to provide indoles of the general structure III-2 or III-4 (where VR¹¹ is a substituted alkyl group) Harrington and Kerr, Synlett, 1047-1048 (1996) is hereby incorporated by reference for such disclosure. In some embodiments, indoles of structure III-3 are reacted with benzyl derivatives (I-5) in warm DMF to yield indoles of structure III-4 where VR¹¹ is a substituted benzyl group. Jacobs et al, J. Med. Chem., v36, 394-409 (1993) is hereby incorporated by reference for such disclosure.

### Further Synthesis of Indole and Indole-Type Compounds

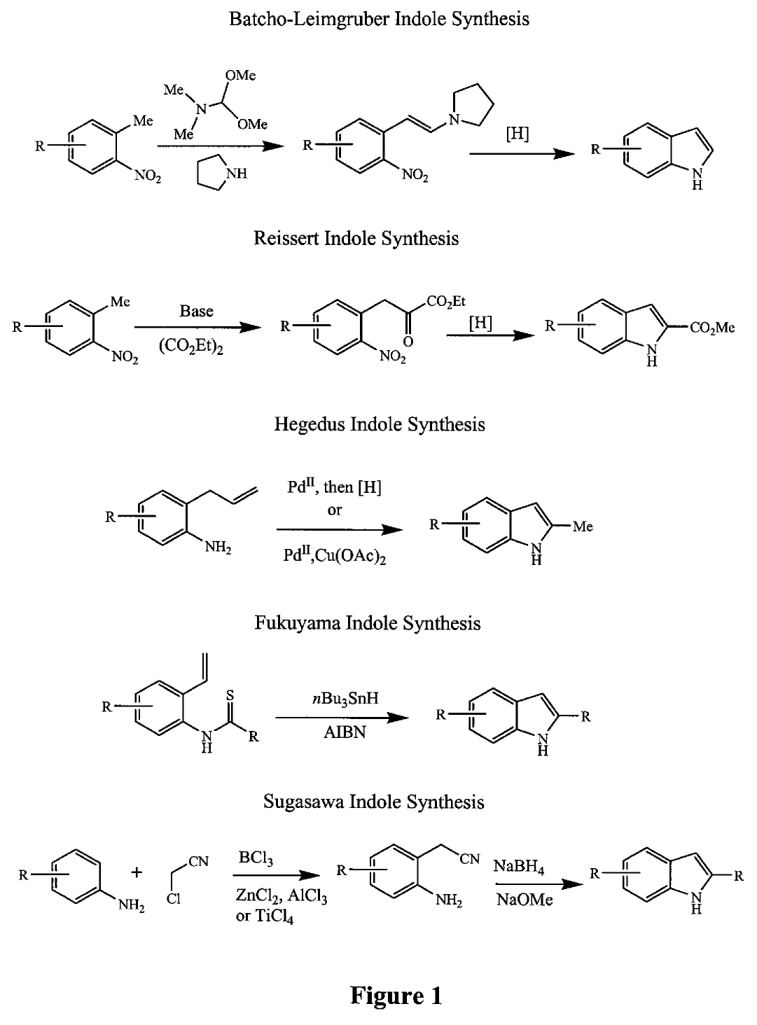

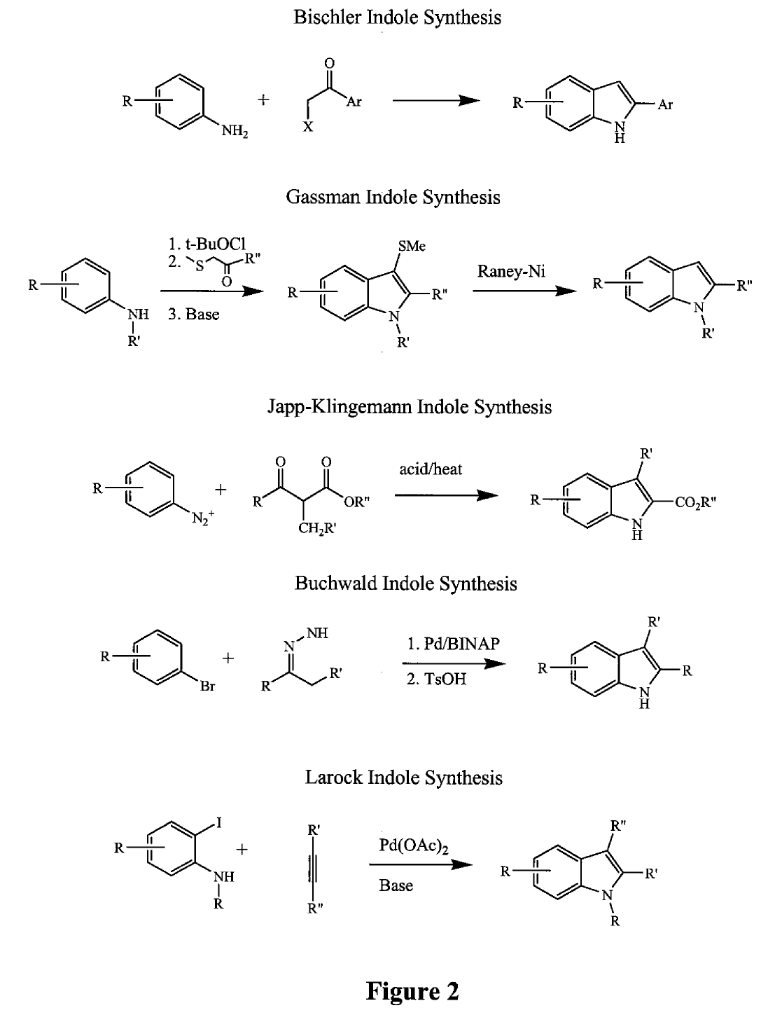

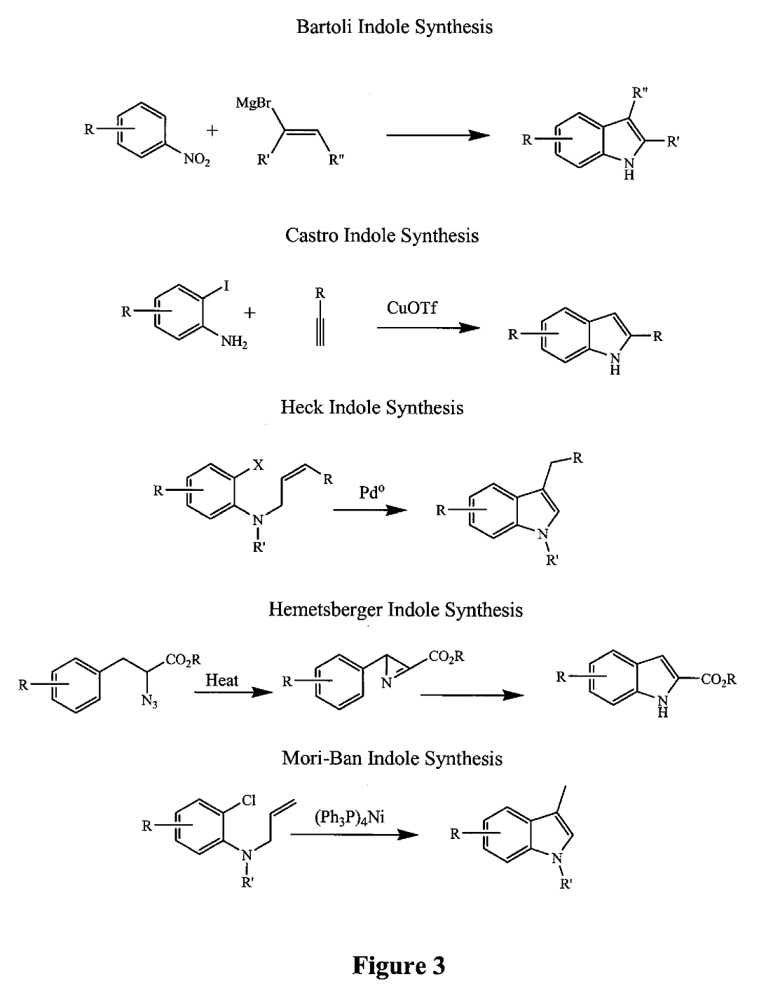

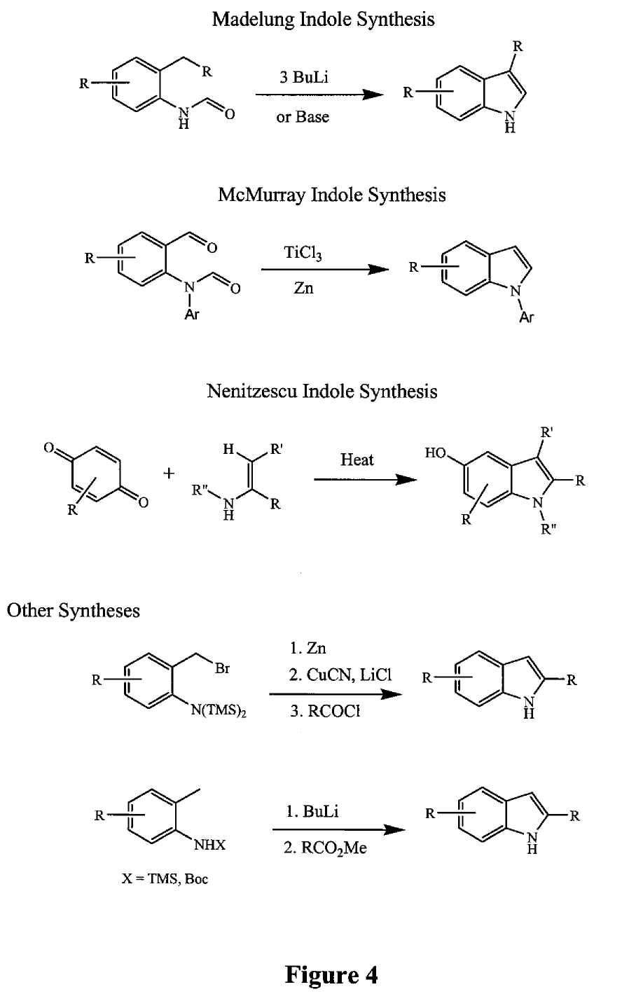

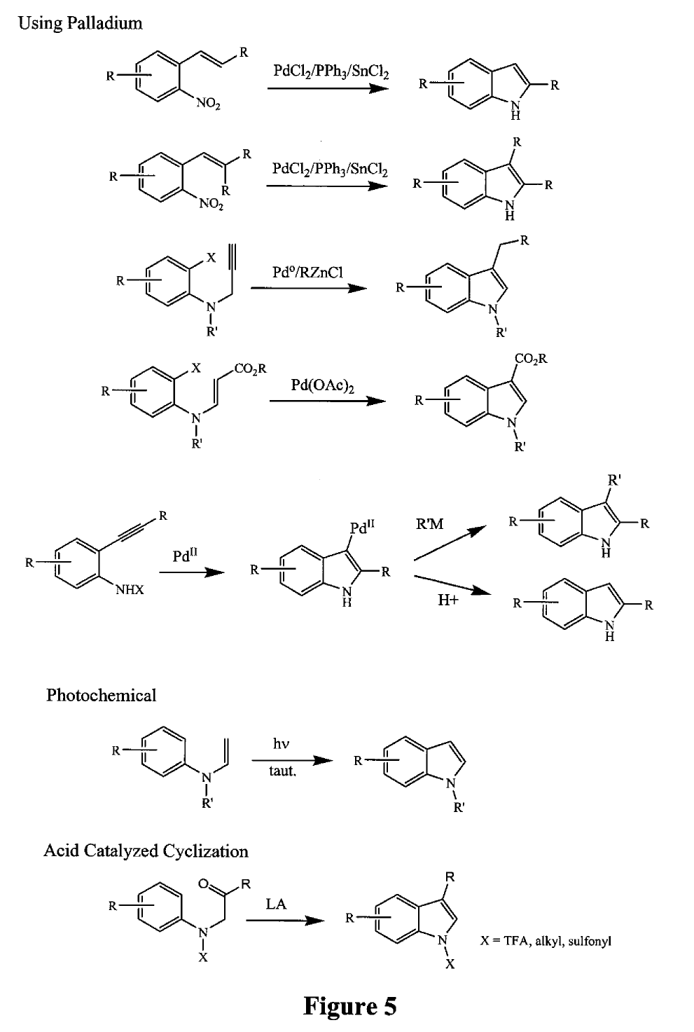

In some embodiments, non-limiting examples of synthetic strategies toward the synthesis of indole compounds of Formulas (A), (B) and (C), include modifications to various syntheses of indoles, including, but not limited to: Batcho-Leimgruber Indole Synthesis, Reissert Indole Synthesis, Hegedus Indole Synthesis, Fukuyama Indole Synthesis, Sugasawa Indole Synthesis, Bischler Indole Synthesis, Gassman Indole Synthesis, Fischer Indole Synthesis, Japp-Klingemann Indole Synthesis, Buchwald Indole Synthesis, Larock Indole Synthesis, Bartoli Indole Synthesis, Castro Indole Synthesis, Hemetsberger Indole Synthesis, Mori-Ban Indole Synthesis, Madelung Indole Synthesis, Nenitzescu Indole Synthesis, and other unnamed reactions. Non-limiting examples of such synthetic methods are shown in FIG. 1, FIG. 2, FIG. 3, FIG. 4, and FIG. 5.

Throughout the specification, groups and substituents thereof are chosen, in various embodiments, to provide stable moieties and compounds.

### Certain Terminology

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the interne can come and go, but equivalent information can be found by searching the interne. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Definition of standard chemistry terms may be found in reference works, including Carey and Sundberg “ADVANCED ORGANIC CHEMISTRY 4^{TH} ED.” Vols. A (2000) and B (2001), Plenum Press, New York. In certain embodiments, conventional methods of mass spectroscopy, NMR, HPLC, protein chemistry, biochemistry, recombinant DNA techniques and pharmacology are employed in the processes herein. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed using conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

As used herein, C₁-Cₓ includes C₁-C₂, C₁-C₃ . . . C₁-Cₓ.

An “alkyl” group refers to an aliphatic hydrocarbon group. Reference to an alkyl group includes “saturated alkyl” and/or “unsaturated alkyl”. An “alkene” group refers to a group consisting of at least two carbon atoms and at least one carbon-carbon double bond, and an “alkyne” group refers to a group consisting of at least two carbon atoms and at least one carbon-carbon triple bond. The alkyl group, whether saturated or unsaturated, includes branched, straight chain, or cyclic groups.

Generally, “alkyl” groups have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). Reference to an “alkyl” group also includes disclosure of a “lower alkyl” having 1 to 6 carbon atoms. The alkyl group of the compounds described herein may be designated as “C₁-C₆ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Disclosure of an alkyl groups includes the disclosure of substituted and/or unsubstituted groups. Depending on the structure, an alkyl group can be a monoradical or a diradical (i.e., an alkylene group).

An “alkoxy” group refers to a (alkyl)O— group, where alkyl is as defined herein.

The term “alkenyl” refers to a type of alkyl group in which the first two atoms of the alkyl group form a double bond that is not part of an aromatic group. That is, an alkenyl group begins with the atoms —C(R)═CR₂, wherein R refers to the remaining portions of the alkenyl group, which may be the same or different. Non-limiting examples of an alkenyl group include —CH═CH₂, —C(CH₃)═CH₂, —CH═CHCH₃ and —C(CH₃)═CHCH₃. The alkenyl groups include branched, straight chain, or cyclic (in which case, it would also be known as a “cycloalkenyl” group) groups. A “lower alkenyl” refers to an alkenyl having 2 to 6 carbons. Disclosure of an alkenyl groups includes the disclosure of a substituted and/or unsubstituted group. Depending on the structure, an alkenyl group can be a monoradical or a diradical (i.e., an alkenylene group).

The term “alkynyl” refers to a type of alkyl group in which the first two atoms of the alkyl group form a triple bond. That is, an alkynyl group begins with the atoms —C≡C—R, wherein R refers to the remaining portions of the alkynyl group. Non-limiting examples of an alkynyl group include —C≡CH, —C≡CCH₃, —C≡CCH₂CH₃ and —C≡CCH₂CH₂CH₃. The “R” portion of the alkynyl moiety may be branched, straight chain, or cyclic. A “lower alkynyl” refers to an alkynyl having 2 to 6 carbons. Disclosure of alkynyl groups includes the disclosure of substituted and/or unsubstituted groups. Depending on the structure, an alkynyl group can be a monoradical or a diradical (i.e., an alkynylene group).

The term “alkylamine” refers to the —N(allyl)ₓH_{y} group, wherein alkyl is as defined herein and x and y are selected from the group x=1, y=1 and x=2, y=0. When x=2, the alkyl groups, taken together with the nitrogen to which they are attached, optionally form a cyclic ring system.

An “amide” is a chemical moiety with formula —C(O)NHR or —NHC(O)R, where R is selected from alkyl, cycloalkyl, aryl, heteroaryl (bonded through a ring carbon) and heteroalicyclic (bonded through a ring carbon). In certain embodiments, an amide is an amino acid or a peptide molecule attached to a compound of Formula (A), (B) or (C), thereby forming a prodrug. Any amine, or carboxyl side chain on the compounds described herein can be amidified. The procedures and specific groups to make such amides include, e.g., those set forth in reference sources such as Greene and Wuts, Protective Groups in Organic Synthesis, 3^{rd} Ed., John Wiley & Sons, New York, N.Y., 1999, which is hereby incorporated by reference for such disclosure.

The term “ester” refers to a chemical moiety with formula —C(═O)OR, where R is selected from the group consisting of alkyl, cycloalkyl, aryl, heteroaryl (bonded through a ring carbon) and heteroalicyclic (bonded through a ring carbon). In various embodiments, any one or more hydroxy, or carboxyl side chain on the compounds described herein are esterified. The procedures and specific groups to make such esters include, e.g., those set forth in reference sources such as Greene and Wuts, Protective Groups in Organic Synthesis, 3^{rd} Ed., John Wiley & Sons, New York, N.Y., 1999, which is hereby incorporated herein by reference for such disclosure.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings disclosed herein are optionally substituted. As disclosed herein rings include monocyclic and/or polycyclic rings.

As used herein, the term “ring system” refers to one, or more than one ring.

The term “membered ring” embraces any cyclic structure. The term “membered” is meant to denote the number of skeletal atoms that constitute the ring. Thus, cyclohexyl, phenyl, pyridine, piperidine, morpholine, piperazine, pyridazine, pyrimidine, pyrazine, pyran and thiopyran are examples of 6-membered rings; and cyclopentyl, pyrrolidine, imidazole, oxazole, thiazole, pyrrole, furan, and thiophene are examples of 5-membered rings.

The term “fused” refers to structures in which two or more rings share one or more bonds.

The term “carbocyclic” or “carbocycle” refers to a ring wherein each of the atoms forming the ring is a carbon atom. Carbocycles includes aryl and cycloalkyl groups. The term thus distinguishes carbocycle from heterocycle (“heterocyclic”) in which the ring backbone contains at least one atom which is different from carbon (i.e a heteroatom). Heterocycle includes heteroaryl and heterocycloalkyl. Carbocycles and heterocycles disclosed herein are optionally substituted.

The term “aromatic” refers to a planar ring having a delocalized π-electron system containing 4n+2 π electrons, where n is an integer. In various embodiments, aromatic rings include rings having five, six, seven, eight, nine, or more than nine atoms. Aromatics disclosed herein are optionally substituted. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings disclosed herein include rings having five, six, seven, eight, nine, or more than nine carbon atoms. Aryl groups are optionally substituted. Examples of aryl groups include, but are not limited to phenyl, and naphthalenyl. Depending on the structure, an aryl group can be a monoradical or a diradical (i.e., an arylene group).

The term “cycloalkyl” refers to a monocyclic or polycyclic non-aromatic radical, wherein each of the atoms forming the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

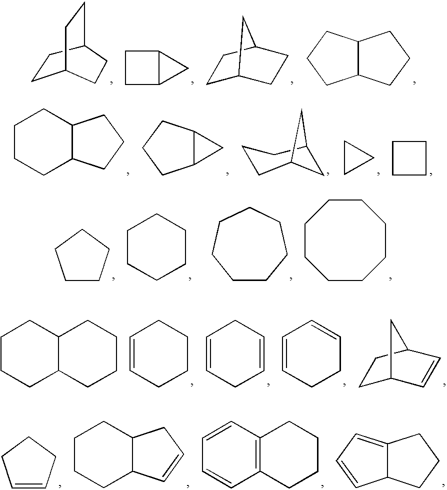

and the like. Monocyclic cycloalkyls include, but are not limited to, cyclopropyl, cyclobutyl, cyclopentyl, cyclohexyl, cycloheptyl, and cyclooctyl. A “lower cycloalkyl” has 3 to 6 ring atoms.

The term “heterocycle” refers to heteroaromatic and heteroalicyclic groups containing one to four ring heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxepanyl, thiepanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinayolinyl, quinoxalinyl, naphthyridinyl, and furopyridinyl. In various embodiments, attachment of the foregoing groups are at a carbon atom (i.e., C-attached) therein or a nitrogen atom therein (i.e., N-attached) where such is possible. For example, in various embodiments, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are monocyclic (not fused) or polycyclic (fused). Illustrative examples of heteroaryl groups include the following moieties:

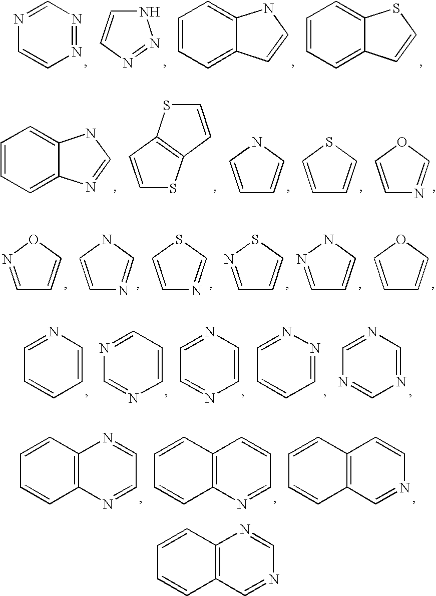

and the like.

A “heteroalicyclic” group or “heterocycloalkyl” group refers to a cycloalkyl group, wherein at least one skeletal ring atom is a heteroatom selected from nitrogen, oxygen and sulfur. In various embodiments, the radicals are with an aryl or heteroaryl. Illustrative examples of heterocycloalkyl groups, also referred to as non-aromatic heterocycles, include:

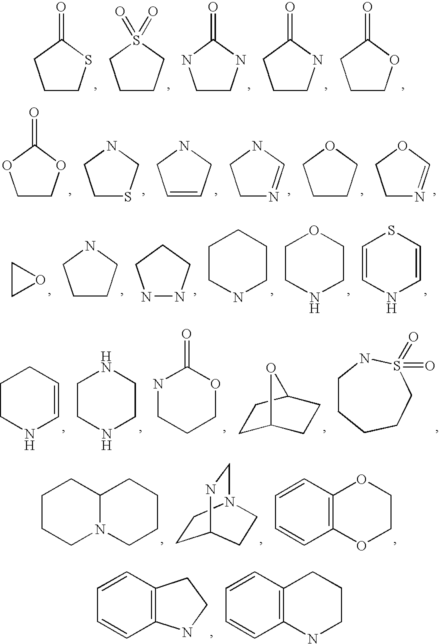

and the like. The term heteroalicyclic also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteratoms) that make up the heterocycloalkyl skeletal atoms of the heterocycloalkyl ring).

The term “halo” or, alternatively, “halogen” means fluoro, chloro, bromo and iodo.

The terms “haloalkyl,” “haloalkenyl,” “haloalkynyl” and “haloalkoxy” include alkyl, alkenyl, alkynyl and alkoxy structures that are substituted with one or more halogens. In embodiments, where more than one halogen is included in the group, the halogens are the same or they are different. The terms “fluoroalkyl” and “fluoroalkoxy” include haloalkyl and haloalkoxy groups, respectively, in which the halo is fluorine.

The terms “heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃. In certain embodiments, heteroalkyl groups have from 1 to 6 carbon atoms (excluding the number of heteroatoms); a “heteroalkenyl” has from 2 to 6 carbons atoms, and a “heteroalkynyl” has from 2 to 6 carbon atoms.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

A “cyano” group refers to a —CN group.

An “isocyanato” group refers to a —NCO group.

A “thiocyanato” group refers to a —CNS group.

An “isothiocyanato” group refers to a —NCS group.

“Mercaptyl” or “sulfanyl” refers to a —S— moiety.

“Acyloxy” refers to a RC(═O)O— group.

“Acyl” refers to a RC(═O)— group.

“Sulfinyl” refers to a —S(═O)— moiety.

“Sulfonyl” refers to a —S(═O)₂— moiety.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

The term “optionally substituted” or “substituted” means that the referenced group substituted with one or more additional group(s). In certain embodiments, the one or more additional group(s) are individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, acyloxy, isocyanato, thiocyanato, isothiocyanato, nitro, haloalkyl, fluoroalkyl, and amino, including mono- and di-substituted amino groups, and the protected derivatives thereof. In certain embodiments, optional substituents are also selected from L^{s}R^{s}, wherein each II is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, —(C₁-C₆alkyl)-, or —(C₂-C₆alkenyl)-; and each R⁸ is independently selected from H, (C₁-C₆alkyl), (C₃-C₈cycloalkyl), aryl, heteroaryl, heteocycloalkyl, or C₁-C₆heteroalkyl. As used herein, “optionally substituted” includes the disclosure of both the “substituted” and “unsubstituted” group.

In some embodiments, the compounds presented herein possess one or more stereocenters. The compounds disclosed herein that possess one or more stereocenter include those with both the R or S configuration at each stereocenter. The compounds presented herein include all diastereomeric, enantiomeric, and epimeric forms as well as the appropriate mixtures thereof.

The methods and formulations described herein include the use of N-oxides, crystalline forms (also known as polymorphs), or pharmaceutically acceptable salts of compounds having the structure of Formula (A), (B) or (C), as well as active metabolites of these compounds having the same type of activity. In some situations, compounds disclosed herein exist as tautomers. All tautomers are included within the scope of the compounds presented herein. In addition, the compounds described exist, in various embodiments, in unsolvated or solvated forms. Solvated forms included those solvated with pharmaceutically acceptable solvents such as water, ethanol, and the like. The solvated forms of the compounds presented herein are also considered to be disclosed herein.

The terms “kit” and “article of manufacture” are used interchangeably herein.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “target protein” refers to a protein or a portion of a protein capable of being bound by a selective binding compound. In certain embodiments, a target protein is FLAP.

As used herein, the term “selective binding compound” refers to a compound that selectively binds to any portion of one or more target proteins.

As used herein, the term “selectively binds” refers to the ability of a selective binding compound to bind to a target protein, such as, for example, FLAP, with greater affinity than it binds to a non-target protein. In certain embodiments, specific binding refers to binding to a target with an affinity that is at least 10, 50, 100, 250, 500, 1000 or more times greater than the affinity for a non-target.

As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a target. For example, a modulator can cause an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target. In certain embodiments, a modulator is an activator, which increases the magnitude of at least one activity of a target. In certain embodiments the presence of a modulator results in an activity that does not occur in the absence of the modulator.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the term “agonist” refers to a compound, the presence of which results in a biological activity of a protein that is the same as the biological activity resulting from the presence of a naturally occurring ligand for the protein, such as, for example, FLAP.

As used herein, the term “antagonist” refers to a compound, the presence of which results in a decrease in the magnitude of a biological activity of a protein. In certain embodiments, the presence of an antagonist results in complete inhibition of a biological activity of a protein, such as, for example, FLAP.

The terms “inhibits”, “inhibiting”, or “inhibitor” of FLAP, as used herein, refer to inhibition of 5-lipoxygenase activating protein activity.

The term “acceptable” with respect to a formulation, composition or ingredient, as used herein, means having no persistent detrimental effect on the general health of the subject being treated.

By “pharmaceutically acceptable,” as used herein, refers a material, such as a carrier or diluent, which does not abrogate the biological activity or properties of the compound, and is relatively nontoxic, i.e., the material is administered to an individual without causing undesirable or substantially undesirable biological effects or interacting or substantially interacting in a deleterious manner with any of the components of the composition in which it is contained.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (A), (B) or (C), and a co-agent, are both administered to a patient simultaneously in the form of a single entity or dosage. The term “non-fixed combination” means that the active ingredients, e.g. a compound of Formula (A), (B) or (C), and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound of Formula (A), (B) or (C) with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “co-administration” or the like, as used herein, are meant to encompass administration of the selected therapeutic agents to a single patient, and are intended to include treatment regimens in which the agents are administered by the same or different route of administration or at the same or different time.

The term “carrier,” as used herein, refers to relatively nontoxic chemical compounds or agents that facilitate the incorporation of a compound into cells or tissues.

The term “diluent” refers to chemical compounds that are used to dilute the compound of interest prior to delivery. In certain instances, diluents are also used to stabilize compounds because they can provide a more stable environment. In some embodiments, salts dissolved in buffered solutions (which also can provide pH control or maintenance) are utilized as diluents, including, but not limited to a phosphate buffered saline solution.

The term “asthma” as used herein refers to any disorder of the lungs characterized by variations in pulmonary gas flow associated with airway constriction of whatever cause (intrinsic, extrinsic, or both; allergic or non-allergic). The term asthma may be used with one or more adjectives to indicate cause.

The term “bone disease,’ as used herein, refers to a disease or condition of the bone, including, but not limited to, inappropriate bone remodeling, loss or gain, osteopenia, osteomalacia, osteofibrosis, and Paget's disease. Garcia, “Leukotriene B4 stimulates osteoclastic bone resorption both in intro and in vivo”, J Bone Miner Res. 11:1619-27 (1996) is hereby incorporated by reference for such disclosure.

The term “cardiovascular disease,” as used herein refers to diseases affecting the heart or blood vessels or both, including but not limited to: arrhythmia; atherosclerosis and its sequelae; angina; myocardial ischemia; myocardial infarction; cardiac or vascular aneurysm; vasculitis, stroke; peripheral obstructive arteriopathy of a limb, an organ, or a tissue; reperfusion injury following ischemia of the brain, heart or other organ or tissue; endotoxic, surgical, or traumaticshock; hypertension, valvular heart disease, heart failure, abnormal blood pressure; shock; vasoconstriction (including that associated with migraines); vascular abnormality, inflammation, insufficiency limited to a single organ or tissue. Lotzer K et al., “The 5-lipoxygenase pathway in arterial wall biology and atherosclerosis”, Biochim Biophys Acta 1736:30-7 (2005); Helgadottir A et al., “The gene encoding 5-lipoxygenase activating protein confers risk of myocardial infarction and stroke’, Nat. Genet. March; 36(3):233-9 (2004); and Heise C E, Evans J F et al., “Characterization of the human cysteinyl leukotriene 2 receptor”, J Biol. Chem. 275(39):30531-6 (2000) are hereby incorporated by reference for such disclosure.

The term “cancer,’ as used herein refers to an abnormal growth of cells which tend to proliferate in an uncontrolled way and, in some cases, to metastasize (spread). The types of cancer include, but is not limited to, solid tumors (such as those of the bladder, bowel, brain, breast, endometrium, heart, kidney, lung, lymphatic tissue (lymphoma), ovary, pancreas or other endocrine organ (thyroid), prostate, skin (melanoma) or hematological tumors (such as the leukemias). Ding X Z et al., “A novel anti-pancreatic cancer agent, LY293111”, Anticancer Drugs. 16(5):467-73 (2005); and Review; Chen X et al., “Overexpression of 5-lipoxygenase in rat and human esophageal adenocarcinoma and inhibitory effects of zileuton and celecoxib on carcinogenesis”, Clin Cancer Res. 10(19):6703-9 (2004) are hereby incorporated by reference for such disclosure.

The term “dermatological disorder,” as used herein refers to a skin disorder. Such dermatological disorders include, but are not limited to, proliferative or inflammatory disorders of the skin such as, atopic dermatitis, bullous disorders, collagenoses, contact dermatitis eczema, Kawasaki Disease, rosacea, Sjogren-Larsso Syndrome, urticaria. Wedi B et al., “Pathophysiological role of leukotrienes in dermatological diseases: potential therapeutic implications”, BioDrugs. 15(11):729-43 (2001) are hereby incorporated by reference for such disclosure.

The terms “fibrosis” or “fibrosing disorder,” as used herein, refers to conditions that follow acute or chronic inflammation and are associated with the abnormal accumulation of cells and/or collagen and include but are not limited to fibrosis of individual organs or tissues such as the heart, kidney, joints, lung, or skin, and includes such disorders as idiopathic pulmonary fibrosis and cryptogenic fibrosing alveolitis. Charbeneau R P et al., “Eicosanoids: mediators and therapeutic targets in fibrotic lung disease”, Clin Sci (Lond). 108(6):479-91 (2005) is hereby incorporated by reference for such disclosure.

The term “iatrogenic” means a leukotriene-dependent or leukotriene-mediated condition, disorder, or disease created or worsened by medical or surgical therapy.

The term “inflammatory disorders” refers to those diseases or conditions that are characterized by one or more of the signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and loss of function (functio laesa, which may be partial or complete, temporary or permanent). Inflammation takes many forms and includes, but is not limited to, inflammation that is one or more of the following: acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative. Inflammatory disorders further include, without being limited to those affecting the blood vessels (polyarteritis, temporarl arteritis); joints (arthritis: crystalline, osteo-, psoriatic, reactive, rheumatoid, Reiter's); gastrointestinal tract (Crohn's Disease, ulcerative colitis); skin (dermatitis); or multiple organs and tissues (systemic lupus erythematosus). Harrison's Principles of Internal Medicine, 16^{th} Edition, Kasper D L, et al, Editors; McGraw-Hill, publishers is hereby incorporated by reference for such disclosure.

The term “interstitial cystitis” refers to a disorder characterized by lower abdominal discomfort, frequent and sometimes painful urination that is not caused by anatomical abnormalites, infection, toxins, trauma or tumors. Bouchelouche K et al., “The cysteinyl leukotrine D4 receptor antagonist montelukast for the treatment of interstitial cystitis”, J Urol 166:1734 (2001) is hereby incorporated by reference for such disclosure.

The term “leukotriene-driven mediators,” as used herein, refers to molecules able to be produced in a patient that may result from excessive production of leukotriene stimulation of cells, such as, by way of example only, LTB₄, LTC₄, LTE₄, cysteinyl leuktorienes, monocyte inflammatory protein (MIP-1α), interleukin-8 (IL-8), interleukin-4 (IL-4), interleukin-13 (IL-13), monocyte chemoattractant protein (MCP-1), soluble intracellular adhesion molecule (sICAM; soluble ICAM), myeloperoxidase (MPO), eosinophil peroxidase (EPO), and general inflammation molecules such as interleukin-6 (Il-6), C-reactive protein (CRP), and serum amyloid A protein (SAA).

The term “leukotriene-related mediators,” as used herein, refers to molecules able to be produced in a patient that may result from excessive production of leukotriene stimulation of cells, such as, by way of example only, LTB₄, LTC₄, LTE₄, cysteinyl leuktorienes, monocyte inflammatory protein (MIP-1α), interleukin-8 (IL-8), interleukin-4 (IL-4), interleukin-13 (IL-13), monocyte chemoattractant protein (MCP-1), soluble intracellular adhesion molecule (sICAM; soluble ICAM), myeloperoxidase (MPO), eosinophil peroxidase (EPO), and general inflammation molecules such as interleukin-6 (Il-6), C-reactive protein (CRP), and serum amyloid A protein (SAA).

The term “leukotriene-dependent”, as used herein, refers to conditions or disorders that would not occur, or would not occur to the same extent, in the absence of one or more leukotrienes.

The term “leukotriene-mediated”, as used herein, refers to refers to conditions or disorders that might occur in the absence of leukotrienes but can occur in the presence of one or more leukotrienes.

The term “leukotriene-responsive patient,” as used herein, refers to a patient who has been identified by either genotyping of FLAP haplotypes, or genotyping of one or more other genes in the leukotriene pathway and/or, by phenotyping of patients either by previous positive clinical response to another leukotriene modulator, including, by way of example only, zileuton (Zyflo™), montelukast (Singulair™), pranlukast (Onon™) zafirlukast (Accolate™), and/or by their profile of leukotriene-driven mediators that indicate excessive leukotriene stimulation of inflammatory cells, as likely to respond favorably to leukotriene modulator therapy.

The terms “neurogenerative disease” or “nervous system disorder,” as used herein, refers to conditions that alter the structure or function of the brain, spinal cord or peripheral nervous system, including but not limited to Alzheimer's Disease, cerebral edema, cerebral ischemia, multiple sclerosis, neuropathies, Parkinson's Disease, those found after blunt or surgical trauma (including post-surgical cognitive dysfunction and spinal cord or brain stem injury), as well as the neurological aspects of disorders such as degenerative disk disease and sciatica. The acronym “CNS” refers to disorders of the central nervous system, i.e., brain and spinal cord. Sugaya K, et al., “New anti-inflammatory treatment strategy in Alzheimer's disease”, Jpn J. Pharmacol. 82(2):85-94 (2000); Yu G L, et al., “Montelukast, a cysteinyl leukotriene receptor-1 antagonist, dose- and time-dependently protects against focal cerebral ischemia in mice”, Pharmacology. 73(1):31-40 (2005); and Zhang W P, et al., “Neuroprotective effect of ONO-1078, a leukotriene receptor antagonist, on focal cerebral ischemia in rats’, Acta Pharmacol Sin. 23(10):871-7 (2002) are hereby incorporated by reference for such disclosure.

The terms “ocular disease” or “ophthalmic disease,” as used herein, refer to diseases which affect the eye or eyes and potentially the surrounding tissues as well. Ocular or ophthalmic diseases include, but are not limited to, conjunctivitis, retinitis, scleritis, uveitis, allergic conjuctivitis, vernal conjunctivitis, pappillary conjunctivitis. Toriyama S., “Effects of leukotriene B4 receptor antagonist on experimental autoimmune uveoretinitis in rats”, Nippon Ganka Gakkai Zasshi. 104(6):396-40 (2000); and Chen F, et al., “Treatment of S antigen uveoretinitis with lipoxygenase and cyclo-oxygenase inhibitors”, Ophthalmic Res. 23(2):84-91 (1991) are hereby incorporated by reference for such disclosure.

The term “respiratory disease,” as used herein, refers to diseases affecting the organs that are involved in breathing, such as the nose, throat, larynx, trachea, bronchi, and lungs. Respiratory diseases include, but are not limited to, asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia. Evans J F, “The Cysteinyl Leukotriene (CysLT) Pathway in Allergic Rhinitis”, Allergology International 54:187-90 (2005); Kemp J P., “Leukotriene receptor antagonists for the treatment of asthma”, IDrugs. 3(4):430-41 (2000); and Riccioni G, et al., “Effect of the two different leukotriene receptor antagonists, montelukast and zafirlukast, on quality of life: a 12-week randomized study”, Allergy Asthma Proc. 25(6):445-8 (2004) are hereby incorporated by reference for such disclosure.

The term “enzymatically cleavable linker,” as used herein refers to unstable or degradable linkages which may be degraded by one or more enzymes.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, in certain instances, enzymes produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. The Pharmacological Basis of Therapeutics, 9th Edition, McGraw-Hill (1996) is hereby incorporated by reference for such disclosure. In certain embodiments, metabolites of the compounds disclosed herein are identified, by way of non-limiting example, by administration of compounds to a host and analysis of tissue samples from the host, or by incubation of compounds with hepatic cells in vitro and analysis of the resulting compounds.

### Pharmaceutical Composition/Formulation

In certain embodiments, provided herein are pharmaceutical compositions comprising a compound of Formula (A), (B) or (C). In some embodiments, pharmaceutical compositions are formulated in a conventional manner using one or more physiologically acceptable carriers comprising excipients and auxiliaries which facilitate processing of the active compounds into preparations which can be used pharmaceutically. In some embodiments, proper formulation is dependent upon the route of administration chosen. In certain embodiments, any technique, carrier, and/or excipient are used. Remington: The Science and Practice of Pharmacy, Nineteenth Ed (Easton, Pa.: Mack Publishing Company (1995); Hoover, John E., Remington's Pharmaceutical Sciences, Mack Publishing Co., Easton, Pa. (1975); Liberman, H. A. and Lachman, L., Eds., Pharmaceutical Dosage Forms, Marcel Decker, New York, N.Y. (1980); and Pharmaceutical Dosage Forms and Drug Delivery Systems, Seventh Ed. (Lippincott Williams & Wilkins 1999), are hereby incorporated by reference for such disclosure.

Provided herein are pharmaceutical compositions that include a compound of Formula (A), and a pharmaceutically acceptable diluent(s), excipient(s), or carrier(s). In certain embodiments, the compounds described herein are administered as pharmaceutical compositions in which compounds of Formula (A), are mixed with other active ingredients, as in combination therapy. Provided herein are pharmaceutical compositions that include a compound of Formula (B), and a pharmaceutically acceptable diluent(s), excipient(s), or carrier(s). In certain embodiments, the compounds described herein are administered as pharmaceutical compositions in which compounds of Formula (B), are mixed with other active ingredients, as in combination therapy. Provided herein are pharmaceutical compositions that include a compound of Formula (C), and a pharmaceutically acceptable diluent(s), excipient(s), or carrier(s). In certain embodiments, the compounds described herein are administered as pharmaceutical compositions in which compounds of Formula (C), are mixed with other active ingredients, as in combination therapy.

A pharmaceutical composition, as used herein, refers to a mixture of a compound of Formula (A), (B) or (C) with other chemical components, such as carriers, stabilizers, diluents, dispersing agents, suspending agents, thickening agents, and/or excipients. In certain embodiments, the pharmaceutical composition facilitates administration of the compound to an organism. In practicing the methods of treatment or use provided herein, therapeutically effective amounts of a compound of Formula (A), (B) or (C) is administered in a pharmaceutical composition to a mammal having a disease or condition to be treated. In a specific embodiment, the mammal is a human. In various embodiments, the therapeutically effective amount varies depending on the severity of the disease, the age and relative health of the subject, the potency of the compound used and other factors. In some embodiments, the compounds are used singly or in combination with one or more therapeutic agents as components of mixtures.

Suitable routes of administration of the compounds and/or compositions described herein include, but are not limited to, intravenous, oral, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In some embodiments, compounds and/or compositions are administered in a local rather than systemic manner, for example, via injection of the compound directly into an organ, often in a depot preparation or sustained release formulation. In some embodiments, long acting formulations are administered by implantation (for example subcutaneously or intramuscularly) or by intramuscular injection. In certain embodiments, the drug is administered in a targeted drug delivery system, for example, in a liposome coated with organ-specific antibody. In some embodiments, the liposomes are targeted to and taken up selectively by the specific organ. In additional embodiments, the drug is provided in the form of a rapid release formulation, in the form of an extended release formulation, or in the form of an intermediate release formulation.

For intravenous injections, compounds of Formula (A) may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations may include aqueous or nonaqueous solutions, preferably with physiologically compatible buffers or excipients.

In some embodiments, pharmaceutical compositions comprising compounds of Formula (A), (B), or (C) are formulated for oral administration by combining the active compounds with pharmaceutically acceptable carriers or excipients. In various embodiments, the selected carrier(s) enable the compounds described herein to be formulated as tablets, powders, pills, dragees, capsules, liquids, gels, syrups, elixirs, slurries, suspensions and the like, for oral ingestion by a patient to be treated.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. In some embodiments, suitable excipients are, e.g., fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In some embodiments, the pharmaceutical composition comprises a disintegrating agent, such as the cross-linked croscarmellose sodium, polyvinylpyrrolidone, agar, or alginic acid or a salt thereof such as sodium alginate.

In some embodiments, dragee cores are provided with suitable coatings. In certain embodiments, concentrated sugar solutions are used to prepare a coating. In some embodiments, the coatings optionally contain gum arabic, talc, polyvinylpyrrolidone, carbopol gel, polyethylene glycol, and/or titanium dioxide, lacquer solutions, and suitable organic solvents or solvent mixtures. Furthermore, dyestuffs or pigments are optionally added to the tablets or dragee coatings for identification or to characterize different combinations of active compound doses.

In certain embodiments, oral pharmaceutical preparations include, by way of non-limiting example, push-fit capsules made of gelatin, as well as soft, sealed capsules made of gelatin and a plasticizer, such as glycerol or sorbitol. In some embodiments, the push-fit capsules contain the active ingredients in admixture with filler such as lactose, binders such as starches, and/or lubricants such as talc or magnesium stearate and, optionally, stabilizers. In certain embodiments, soft capsules contain the active compounds dissolved or suspended in suitable liquids, such as fatty oils, liquid paraffin, or liquid polyethylene glycols. Stabilizers are optionally added. In certain embodiments, formulations for oral administration are in dosages suitable for such administration.

For buccal or sublingual administration, the compositions may take the form of tablets, lozenges, or gels formulated in a conventional manner. Parental injections may involve bolus injection or continuous infusion. Formulations for injection may be presented in unit dosage form, e.g., in ampoules or in multi-dose containers, with an added preservative. In some embodiments, the pharmaceutical composition that includes a compound of Formula (A), (B) or (C) is in a form suitable for parenteral injection as a sterile suspensions, solutions or emulsions in oily or aqueous vehicles. In some embodiments, the parenteral formulation contains formulatory agents such as suspending, stabilizing and/or dispersing agents. In certain embodiments, pharmaceutical formulations for parenteral administration include aqueous solutions of the active compounds in water-soluble form. In some embodiments, suspensions of the active compounds are prepared as appropriate oily injection suspensions. Suitable lipophilic solvents or vehicles include, by way of non-limiting example, fatty oils such as sesame oil, or synthetic fatty acid esters, such as ethyl oleate or triglycerides, or liposomes. In some embodiments, aqueous injection suspensions contain substances which increase the viscosity of the suspension, such as sodium carboxymethyl cellulose, sorbitol, or dextran. Optionally, the suspension also contains suitable stabilizers or agents which increase the solubility of the compounds to allow for the preparation of highly concentrated solutions. In certain embodiments, the active ingredient is in powder form for constitution with a suitable vehicle, e.g., sterile pyrogen-free water, before use.

In some embodiments, the compounds of Formula (A), (B) or (C) are administered topically. In certain embodiments, topical formulations comprise a compound that is formulated into any suitable topically administrable composition including, by way of non-limiting example, solutions, suspensions, lotions, gels, pastes, medicated sticks, balms, creams or ointments. In some embodiments, topical formulations optionally comprise solubilizers, stabilizers, tonicity enhancing agents, buffers and/or preservatives.

In certain embodiments, formulations suitable for transdermal administration of the compounds of Formula (A), (B) or (C) employ, e.g., transdermal delivery devices and/or transdermal delivery patches. In some embodiments, formulations suitable for transdermal administration and/or transdermal delivery devices and/or transdermal delivery patches comprise lipophilic emulsions or buffered, aqueous solutions, dissolved and/or dispersed in a polymer or an adhesive. In some embodiments, patches are constructed for continuous, pulsatile, or on demand delivery of pharmaceutical agents. In some embodiments, transdermal delivery of the compounds of Formula (A), (B) and/or (C) is accomplished by the use of iontophoretic patches and the like. In some embodiments, transdermal patches provide controlled delivery of the compounds of Formula (A), (B) and/or (C). In certain embodiments, the rate of absorption is slowed by using rate-controlling membranes or by trapping the compound within a polymer matrix or gel. In alternate embodiments, absorption enhancers are used to increase absorption. In certain embodiments, absorption enhancers or carriers include absorbable pharmaceutically acceptable solvents to assist passage through the skin. In some embodiments, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In certain embodiments, the compounds described herein are formulated in a manner suitable for inhalation. In some embodiments, formulations suitable for administration by inhalation include, by way of non-limiting example, aerosols, mists and powders. In some embodiments, pharmaceutical compositions of the compounds described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. In some embodiments, capsules and cartridges of, such as, by way of example only, gelatin for use in an inhaler or insufflator are formulated containing a powder mix of the compound and a suitable powder base such as lactose or starch.

In some embodiments, the compounds described herein, including compounds of Formulas (A), (B) and (C), are formulated in a manner suitable for rectal administration. Formulations suitable for rectal administration include, by way of non-limiting example, enemas, rectal gels, rectal foams, rectal aerosols, suppositories, jelly suppositories, or retention enemas. In some embodiments, formulations suitable for rectal administration comprise conventional suppository bases such as cocoa butter or other glycerides, as well as synthetic polymers such as polyvinylpyrrolidone, PEG, and the like. In specific embodiments, suppositories include, by way of non-limiting example, a low-melting wax such as, but not limited to, a mixture of fatty acid glycerides, optionally in combination with cocoa butter is first melted.

In certain embodiments, pharmaceutical compositions are formulated in a conventional manner using one or more physiologically acceptable carriers including excipients and auxiliaries which facilitate processing of the active compounds into preparations which can be used pharmaceutically. In certain embodiments, proper formulation is dependent upon the route of administration chosen. In various embodiments, any technique, carrier, and/or excipients is used to prepare or in the formulations described herein. In various embodiments, pharmaceutical compositions or formulations described herein are manufactured in any manner, such as, by way of example only, by means of conventional mixing, dissolving, granulating, dragee-making, levigating, emulsifying, encapsulating, entrapping or compression processes.

In certain embodiments, the pharmaceutical compositions described herein comprise at least one compound of any of Formulas (A), (B) and/or (C) and at least one pharmaceutically acceptable carrier, diluent or excipient. In some embodiments, the pharmaceutical compositions comprise at least one compound of Formulas (A), (B) and/or (C) in free-acid or free-base form, or in a pharmaceutically acceptable salt form. In some embodiments, the methods and pharmaceutical compositions described herein include the use of N-oxides, crystalline forms (also known as polymorphs), as well as active metabolites of these compounds having the same type of activity. As discussed herein, in some embodiments, the compounds described herein exist as a tautomer of a compound described herein. It is to be understood that all tautomers are included within the scope of the compounds described herein. Furthermore, it is to be understood that the compounds described herein exist in unsolvated or solvated forms. In certain embodiments, solvated forms of the compounds herein are solvated with pharmaceutically acceptable solvents such as, by way of non-limiting example, water, ethanol, and the like. Accordingly, the solvated forms of the compounds presented herein are also considered to be disclosed herein. In certain embodiments, the pharmaceutical compositions described herein optionally comprise other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions optionally comprise other therapeutically valuable substances.

Methods for the preparation of compositions that include the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions that include a compound, or a solution containing liposomes, micelles, or nanoparticles that include a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

In certain embodiments, a composition described herein that includes those in the form of a liquid. In some embodiments, liquids described herein include compositions wherein the agents are present in solution, in suspension or both. In some embodiments, when a composition described herein is administered as a solution or suspension a first portion of the agent is present in solution and a second portion of the agent is present in particulate form, in suspension in a liquid matrix. In some embodiments, a liquid composition includes a gel formulation. In certain embodiments, the liquid composition is aqueous.

In some embodiments, aqueous suspension optionally comprise one or more polymers as suspending agents. In certain embodiments, such polymers include water-soluble polymers such as cellulosic polymers, e.g., hydroxypropyl methylcellulose, and water-insoluble polymers such as cross-linked carboxyl-containing polymers. In some embodiments, compositions described herein optionally comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, compositions described herein include solubilizing agents to aid in the solubility of a compound described herein. In some embodiments, the term “solubilizing agent” includes agents that result in formation of a micellar solution or a true solution of the agent. In certain embodiments, a composition described herein includes a solubilizing agent that is a nonionic surfactant such as, for example, polysorbate 80, and/or ophthalmically acceptable glycols, polyglycols, e.g., polyethylene glycol 400, and glycol ethers.

In certain embodiments, compositions described herein optionally include one or more pH adjusting agents or buffering agents. In some embodiments, pH adjusting agents include acids (e.g., acetic, boric, citric, lactic, phosphoric and hydrochloric acids) and bases (e.g., sodium hydroxide, sodium phosphate, sodium borate, sodium citrate, sodium acetate, sodium lactate and tris-hydroxymethylaminomethane). In certain embodiments, buffers include, by way of non-limiting example, citrate/dextrose, sodium bicarbonate and ammonium chloride. In certain embodiments, such acids, bases and buffers are included in an amount required to maintain pH of the composition in an acceptable range.

In certain embodiments, compositions described herein include one or more salts in an amount required to bring osmolality of the composition into an acceptable range. In some embodiments, salts include, by way of non-limiting example, those having sodium, potassium or ammonium cations and chloride, citrate, ascorbate, borate, phosphate, bicarbonate, sulfate, thiosulfate or bisulfate anions. In specific embodiments, suitable salts include, by way of non-limiting example, sodium chloride, potassium chloride, sodium thiosulfate, sodium bisulfite and ammonium sulfate.

In some embodiments, compositions described herein include one or more preservatives. In certain embodiments, the preservatives are suitable for inhibiting microbial activity. In some embodiments, preservatives include, by way of non-limiting example, mercury-containing substances such as merfen and thiomersal; stabilized chlorine dioxide; and quaternary ammonium compounds such as benzalkonium chloride, cetyltrimethylammonium bromide and cetylpyridinium chloride.

In certain embodiments, compositions described herein include one or more surfactants to enhance physical stability or for other purposes. In some embodiments, surfactants include non-ionic surfactants such as, by way of non-limiting example, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In some embodiments, compositions described herein include one or more antioxidants to enhance chemical stability where required. In certain embodiments, antioxidants include, by way of example only, ascorbic acid and sodium metabisulfite.

In certain embodiments, aqueous suspension compositions are packaged in single-dose non-re-closable containers. In alternative embodiments, multiple-dose re-closable containers are used. In specific embodiments, a preservative is included in a composition contained in a re-closable container.

In certain embodiments, other delivery systems for hydrophobic pharmaceutical compounds are employed. For example, liposomes and emulsions are utilized for the delivery vehicles or carriers for compounds (e.g., hydrophobic compounds) described herein. In certain organic solvents such as N-methylpyrrolidone are employed. In some embodiments, compounds described herein are delivered using a sustained-release system, such as semipermeable matrices of solid hydrophobic polymers containing the therapeutic agent. In some embodiments, sustained-release capsules release the compounds for a few weeks up to over 100 days. In certain embodiments, depending on the chemical nature and the biological stability of the therapeutic reagent, additional strategies for protein stabilization are employed.

In certain embodiments, the formulations described herein comprise antioxidants, metal chelating agents, thiol containing compounds, other general stabilizing agents and combinations thereof. Examples of stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (1) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Methods of Dosing and Treatment Regimens

In some embodiments, the compounds of Formula (A), (B) or (C) are used in the preparation of medicaments for the treatment of leukotriene-dependent or leukotriene mediated diseases or conditions. In some embodiments, a method for treating any of the diseases or conditions described herein in a subject in need of such treatment, involves administration of at least one compound of Formula (A), (B) or (C), or a pharmaceutically acceptable salt, pharmaceutically acceptable N-oxide, pharmaceutically active metabolite, pharmaceutically acceptable prodrug, or pharmaceutically acceptable solvate thereof. In certain embodiments, the at least one compound of Formula (A), (B) or (C) is administered in a therapeutically effective amount. In certain embodiments, the at least one compound of Formula (A), (B) or (C) is administered as a pharmaceutical composition comprising at least one compound of Formula (A), (B), or (C).

In certain embodiments, the compounds described herein or the compositions thereof are administered for prophylactic and/or therapeutic treatments. In certain therapeutic applications, the compounds described herein or the compositions thereof are administered to a patient already suffering from a disease or condition, in an amount sufficient to cure or at least partially arrest the symptoms of the disease or condition. In certain embodiments, amounts effective for theses uses will depend on the severity and course of the disease or condition, previous therapy, the patient's health status, weight, and response to the drugs, and the judgment of the treating physician. In some embodiments, therapeutically effective amounts are determined by any method, including, by way of non-limiting example, by a dose escalation clinical trial.

In certain prophylactic applications, compounds described herein and compositions thereof are administered to a patient susceptible to or otherwise at risk of a particular disease, disorder or condition. In certain embodiments, the amount of compound administered is defined to be a “prophylactically effective amount or dose.” In certain embodiments, the precise amounts also depend on the patient's state of health, weight, and the like. In certain embodiments, effective amounts for this use depend on the severity and course of the disease, disorder or condition, previous therapy, the patient's health status and response to the drugs, and the judgment of the treating physician. In some embodiments, the effective amounts are determined in any manner, including, e.g., a dose escalation clinical trial.

In some embodiments, upon the doctor's discretion the administration of the compounds is administered chronically, that is, for an extended period of time, including throughout the duration of the patient's life in order to ameliorate or otherwise control or limit the symptoms of the patient's disease or condition.

In some embodiments (e.g., wherein a patient's status does not substantially improve), upon the doctor's discretion the administration of the compounds are given continuously. In certain embodiments, the dose of drug being administered is temporarily reduced or temporarily suspended for a certain length of time (i.e., a “drug holiday”). In various embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. In some embodiments, the dose reduction during a drug holiday is from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In certain embodiments, once improvement of the patient's conditions has occurred, a maintenance dose is administered if necessary and/or desired. In certain embodiments, the dosage or the frequency of administration, or both, is reduced, as a function of the symptoms, to a level at which the improvements in the disease, disorder or condition is retained. In certain embodiments, patients are given intermittent treatment on a long-term basis upon any recurrence of symptoms.

In certain embodiments, the amount of a compound administered that corresponds to an effective amount varies depending upon factors such as the particular compound, disease condition and its severity, the identity (e.g., weight) of the subject or host in need of treatment. In some embodiments, however, doses employed for adult human treatment are in the range of 0.02-5000 mg per day, and in some cases, 1-1500 mg per day. In various embodiments, the desired dose is conveniently presented in a single dose or as divided doses administered simultaneously (or over a short period of time) or at appropriate intervals, for example as two, three, four or more sub-doses per day.

In certain embodiments, the pharmaceutical compositions described herein are in unit dosage forms suitable for single administration of precise dosages. In unit dosage form, the formulation is divided into unit doses containing appropriate quantities of one or more compound. In some embodiments, the unit dosage is in the form of a package containing discrete quantities of the formulation. Non-limiting examples are packaged tablets or capsules, and powders in vials or ampoules. In certain embodiments, aqueous suspension compositions are packaged in single-dose non-reclosable containers. In alternative embodiments, multiple-dose reclosable containers are used, in which case it the composition optionally comprises a preservative. In certain embodiments, formulations for parenteral injection are contained within units including, by way of non-limiting example, ampoules, or multi-dose containers. In such embodiments, the formulations/compositions comprise an optional preservative.

In certain embodiments, the daily dosages appropriate for the compounds described herein, such as compounds of Formula (A), (B) or (C), are from about 0.01 to 2.5 mg/kg per body weight. In certain embodiments, the daily dosage in the larger mammal, including, but not limited to, humans, is in the range from about 0.5 mg to about 100 mg, conveniently administered in divided doses, including, but not limited to, up to four times a day or in extended release form. In some embodiments, the unit dosage forms for oral administration include from about 1 to 50 mg active ingredient. It is to be understood, however, that in some embodiments, the dosage amounts vary for a number of reasons and considerable excursions from these recommended values are within the scope of the disclosed invention. In certain embodiments, dosages are altered depending on a number of variables such as, but in no way limited to, the activity of the compound used, the disease or condition to be treated, the mode of administration, the requirements of the individual subject, the severity of the disease or condition being treated, and the judgment of the practitioner.

In certain embodiments, toxicity and therapeutic efficacy of therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. In certain embodiments, compounds exhibiting high therapeutic indices are utilized in the methods and compositions described herein. In some embodiments, the data obtained from cell culture assays and animal studies are used in formulating a range of dosages for use in human. In some embodiments, the dosage of a compound described herein lies within a range of circulating concentrations that include the ED₅₀ with minimal toxicity. In certain embodiments, the dosage varies within this range depending upon the dosage form employed and the route of administration utilized.

Use of FLAP Modulators to Prevent and/or Treat Leukotriene-Dependent or Leukotriene Mediated Diseases or Conditions

In certain embodiments, provided herein are methods of treating a leukotriene or leukotriene dependent or leukotriene mediated disease or condition with a compound of Formula (A), (B) or (C) or a composition comprising a compound of Formula (A), (B), or (C). It is to be understood that such methods and/or compositions include active metabolites, pharmaceutically acceptable solvates, pharmaceutically acceptable salts, pharmaceutically acceptable N-oxides, or pharmaceutically acceptable prodrugs of compounds of Formula (A), (B) or (C). In certain embodiments, the therapy of leukotriene-dependent or leukotriene mediated diseases or conditions is designed to modulate the activity of FLAP. In certain embodiments, the modulation of FLAP includes, by way of example only, inhibiting or antagonizing FLAP activity. In specific embodiments, a FLAP inhibitor is administered in order to decrease synthesis of leukotrienes within the individual. In some embodiments, a FLAP inhibitor is administered in order to downregulate or decrease the expression or availability of the FLAP mRNA or specific splicing variants of the FLAP mRNA. In certain embodiments, downregulation or decreasing expression or availability of a native FLAP mRNA or of a particular splicing variant minimizes the expression or activity of a defective nucleic acid or the particular splicing variant and thereby minimizes the impact of the defective nucleic acid or the particular splicing variant.

In accordance with one aspect, compositions and methods described herein include compositions and methods for treating, preventing, reversing, halting or slowing the progression of leukotriene-dependent or leukotriene mediated diseases or conditions once it becomes clinically evident, or treating the symptoms associated with or related to leukotriene-dependent or leukotriene mediated diseases or conditions, by administering to the subject a compound of Formula (A), (B) or (C), or pharmaceutical composition or medicament which includes a compound of Formula (A), (B) or (C). In certain embodiments, the subject treated already has a leukotriene-dependent or leukotriene mediated disease or condition at the time of administration. In some embodiments, subjected treated is at risk of developing a leukotriene-dependent or leukotriene mediated disease or condition.

In certain embodiments, the activity of 5-lipoxygenase activating protein in a mammal is directly or indirectly modulated by the administration of (at least once) an effective amount of at least one compound of Formula (A), (B) or (C), or pharmaceutical composition or medicament which includes a compound of Formula (A), (B) or (C), to a mammal. In some embodiments, such modulation includes, but is not limited to, reducing and/or inhibiting the activity of 5-lipoxygenase activating protein. In certain embodiments, the activity of leukotrienes in a mammal is directly or indirectly modulated, including reducing and/or inhibiting, by the administration of (at least once) an effective amount of at least one compound of Formula (A), (B) or (C), or pharmaceutical composition or medicament which includes a compound of Formula (A) (B) or (C), to a mammal. In certain embodiments, such modulation includes, but is not limited to, reducing and/or inhibiting the activity of 5-lipoxygenase activating protein.

In certain embodiments, prevention and/or treatment leukotriene-dependent or leukotriene mediated diseases or conditions includes administering to a mammal at least once an effective amount of at least one compound of Formula (A), (B) or (C), or pharmaceutical composition or medicament which includes a compound of Formula (A), (B) or (C). In some embodiments, the prevention and/or treatment of inflammation diseases or conditions includes administering to a mammal at least once an effective amount of at least one compound of Formula (A), (B) or (C), or pharmaceutical composition or medicament which includes a compound of Formula (A), (B) or (C). In some embodiments, leukotriene-dependent or leukotriene mediated diseases or conditions that are treated by a method that includes administering to a mammal at least once an effective amount of at least one compound of Formula (A), (B) or (C), or pharmaceutical composition or medicament which includes a compound of Formula (A), (B) or (C), include, but are not limited to, bone diseases and disorder, cardiovascular diseases and disorders, inflammatory diseases and disorders, dermatological diseases and disorders, ocular diseases and disorders, cancer and other proliferative diseases and disorders, respiratory diseases and disorder, and non-cancerous disorders.

By way of example only, included in the prevention/treatment methods described herein are methods for treating respiratory diseases that include administering to the mammal at least once an effective amount of at least one compound of Formula (A), (B) or (C), or pharmaceutical composition or medicament which includes a compound of Formula (A), (B) or (C). In some embodiments, the respiratory disease is asthma. Riccioni et al, Ann. Clin. Lab. Sci., v34, 379-387 (2004) is hereby incorporated by reference for such disclosure. In certain embodiments, the respiratory disease is selected from, by way of non-limiting example, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, allergic rhinitis, vascular responses, endotoxin shock, fibrogenesis, pulmonary fibrosis, allergic diseases, chronic inflammation, and adult respiratory distress syndrome.

By way of example only, included in such treatment methods are methods for preventing chronic obstructive pulmonary disease that include administering to the mammal at least once an effective amount of at least one compound of Formula (A), (B) or (C), or pharmaceutical composition or medicament which includes a compound of Formula (A), (B) or (C). In addition, chronic obstructive pulmonary disease includes, but is not limited to, chronic bronchitis or emphysema, pulmonary hypertension, interstitial lung fibrosis and/or airway inflammation and cystic fibrosis.

By way of example only, included in such treatment methods are methods for preventing increased mucosal secretion and/or edema in a disease or condition that include administering to the mammal at least once an effective amount of at least one compound of Formula (A), (B) or (C), or pharmaceutical composition or medicament which includes a compound of any of Formula (A), (B) or (C).

By way of example only, included in the prevention/treatment methods described herein are methods for preventing or treating vasoconstriction, atherosclerosis and its sequelae myocardial ischemia, myocardial infarction, aortic aneurysm, vasculitis and stroke that include administering at least once to the mammal an effective amount of at least one compound of Formula (A), (B) or (C), or pharmaceutical composition or medicament which includes a compound of Formula (A), (B) or (C). Bala et al, Trends in Immunol., v25, 315-322, 2004; and Mehrabian et al, Curr. Opin. Lipidol., v14, 447-457 (2003) is hereby incorporated by reference for such disclosure.

By way of example only, included in the prevention/treatment methods described herein are methods for reducing cardiac reperfusion injury following myocardial ischemia and/or endotoxic shock that include administering at least once to the mammal an effective amount of at least one compound of Formula (A), (B) or (C), or pharmaceutical composition or medicament which includes a compound of Formula (A), (B) or (C).

By way of example only, included in the prevention/treatment methods described herein are methods for reducing the constriction of blood vessels in a mammal that include administering at least once to the mammal an effective amount of at least one compound of Formula (A), (B) or (C), or pharmaceutical composition or medicament which includes a compound of Formula (A), (B) or (C).

By way of example only, included in the prevention/treatment methods described herein are methods for lowering or preventing an increase in blood pressure of a mammal that include administering at least once to the mammal an effective amount of at least one compound of Formula (A), (B) or (C), or pharmaceutical composition or medicament which includes a compound of Formula (A), (B) or (C).

By way of example only, included in the prevention/treatment methods described herein are methods for preventing eosinophil and/or basophil and/or dendritic cell and/or neutrophil and/or monocyte recruitment that include administering at least once to the mammal an effective amount of at least one compound of Formula (A), (B) or (C), or pharmaceutical composition or medicament which includes a compound of Formula (A), (B) or (C).

By way of example only, included in the prevention/treatment methods described herein are methods for the prevention or treatment of abnormal bone remodeling, loss or gain, including diseases or conditions as, by way of example, osteopenia, osteoporosis, Paget's disease, cancer and other diseases that include administering at least once to the mammal an effective amount of at least one compound of Formula (A), (B) or (C), or pharmaceutical composition or medicament which includes a compound of Formula (A), (B) or (C).

By way of example only, included in the prevention/treatment methods described herein are methods for preventing ocular inflammation and allergic conjunctivitis, vernal keratoconjunctivitis, and papillary conjunctivitis that include administering at least once to the mammal an effective amount of at least one compound of Formula (A), (B) or (C), or pharmaceutical composition or medicament which includes a compound of Formula (A), (B) or (C). Lambiase et al, Arch. Opthalmol., v121, 615-620 (2003) is hereby incorporated by reference for such disclosure.

By way of example only, included in the prevention/treatment methods described herein are methods for preventing CNS disorders that include administering at least once to the mammal an effective amount of at least one compound of Formula (A), (B) or (C), or pharmaceutical composition or medicament which includes a compound of Formula (A), (B) or (C). CNS disorders include, but are not limited to, multiple sclerosis, Parkinson's disease, Alzheimer's disease, stroke, cerebral ischemia, retinal ischemia, post-surgical cognitive dysfunction, migraine, peripheral neuropathy/neuropathic pain, spinal cord injury, cerebral edema and head injury.

By way of example only, included in the prevention/treatment methods described herein are methods for the treatment of cancer that include administering at least once to the mammal an effective amount of at least one compound of Formula (A), (B) or (C), or pharmaceutical composition or medicament which includes a compound of Formula (A), (B) or (C). In certain embodiments, the cancer is selected from, by way of non-limiting example, pancreatic cancer and other solid or hematological tumors. Poff and Balazy, Curr. Drug Targets Inflamm. Allergy, v3, 19-33 (2004); and Steele et al, Cancer Epidemiology & Prevention, v8, 467-483 (1999) are hereby incorporated by reference for such disclosure.

By way of example only, included in the prevention/treatment methods described herein are methods for preventing endotoxic shock and septic shock that include administering at least once to the mammal an effective amount of at least one compound of Formula (A), (B) or (C), or pharmaceutical composition or medicament which includes a compound of Formula (A), (B) or (C).

By way of example only, included in the prevention/treatment methods described herein methods for preventing rheumatoid arthritis and osteoarthritis that include administering at least once to the mammal an effective amount of at least one compound of Formula (A), (B) or (C), or pharmaceutical composition or medicament which includes a compound of Formula (A), (B) or (C).

By way of example only, included in the prevention/treatment methods described herein are methods for preventing increased GI diseases that include administering at least once to the mammal an effective amount of at least one compound of Formula (A), (B) or (C), or pharmaceutical composition or medicament which includes a compound of Formula (A), (B) or (C). Such GI diseases include, by way of example only, inflammatory bowel disease (IBD), such as, by way of example only, colitis and Crohn's disease.

By way of example only, included in the prevention/treatment methods described herein are methods for the reduction of inflammation while also preventing transplant rejection or preventing or treating tumors or accelerating the healing of wounds that include administering at least once to the mammal an effective amount of at least one compound of Formula (A), (B) or (C), or pharmaceutical composition or medicament which includes a compound of Formula (A), (B) or (C).

By way of example only, included in the prevention/treatment methods described herein are methods for the prevention or treatment of rejection or dysfunction in a transplanted organ or tissue that include administering at least once to the mammal an effective amount of at least one compound of Formula (A), (B) or (C), or pharmaceutical composition or medicament which includes a compound of Formula (A), (B) or (C).

By way of example only, included in the prevention/treatment methods described herein are methods for treating type II diabetes that include administering to at least once to the mammal an effective amount of at least one compound of Formula (A), (B) or (C), or pharmaceutical composition or medicament which includes a compound of Formula (A), (B) or (C).

By way of example only, included in the prevention/treatment methods described herein are methods for treating inflammatory responses of the skin that include administering at least once to the mammal an effective amount of at least one compound of Formula (A), (B) or (C), or pharmaceutical composition or medicament which includes a compound of Formula (A), (B) or (C). Such inflammatory responses of the skin include, by way of example, psoriasis, dermatitis, contact dermatitis, eczema, urticaria, rosacea, wound healing and scarring. In another aspect are methods for reducing psoriatic lesions in the skin, joints, or other tissues or organs, which include administering at least once to the mammal an effective amount of at least one compound of Formula (A), (B) or (C), or pharmaceutical composition or medicament which includes a compound of Formula (A), (B) or (C).

By way of example only, included in the prevention/treatment methods described herein are methods for the treatment of cystitis, including, by way of example only, interstitial cystitis, that include administering at least once to the mammal an effective amount of at least one compound of Formula (A), (B) or (C), or pharmaceutical composition or medicament which includes a compound of Formula (A), (B) or (C).

By way of example only, included in the prevention/treatment methods described herein are methods for the treatment of metabolic syndromes such as Familial Mediterranean Fever comprising administering at least once to the mammal an effective amount of at least one compound of Formula (A), (B) or (C), or pharmaceutical composition or medicament which includes a compound of Formula (A), (B) or (C).

### Combination Treatments

In certain embodiments, at least one compound of Formula (A), (B) or (C) is administered in combination with another therapeutic agent. If one of the side effects experienced by a patient upon receiving one of the compounds herein is inflammation, then it is, in some embodiments, appropriate to administer an anti-inflammatory agent in combination with the initial therapeutic agent. In certain embodiments, a compound described herein is administered or combined in a composition together with an adjuvant, e.g., an agent that enhances the therapeutic effectiveness of one of the compounds described herein. In some embodiments, the adjuvant has a minimal therapeutic benefit, but in combination with a compound described herein, the overall therapeutic benefit to the patient is enhanced. In certain embodiments, a compound described herein is administered or combined in a composition together with another therapeutic agent that also has therapeutic benefit. It is to be understood that as used herein, agents that are administered together includes administering the agents in the same composition, at the same time or during the course of a single treatment regimen (e.g., sequentially). In some embodiments, in a treatment for asthma involving administration of one of the compounds described herein, increased therapeutic benefit results from providing the patient with other therapeutic agents or therapies for the treatment of asthma. Combination treatments include those that provide an overall benefit to the patient that is, e.g., additive or synergistic.

In certain embodiments, the therapeutically effective dosages of agents used in treatment disclosed herein vary when such agents are administered in combination. Any suitable method for experimentally determining therapeutically effective dosages of drugs and other agents for use in combination treatment regimens is utilized. In certain embodiments, metronomic dosing, i.e., providing more frequent, lower doses in order to minimize toxic side effects, is utilized in a method described herein. In some embodiments, a combination treatment regimen encompasses treatment regimens in which administration of a FLAP inhibitor described herein is initiated prior to, during, or after treatment with a second agent described above, and continues until any time during treatment with the second agent or after termination of treatment with the second agent. Some embodiments also include treatments in which a FLAP inhibitor described herein and a second agent being used in combination are administered simultaneously or at different times and/or at decreasing or increasing intervals during the treatment period. In some embodiments, combination treatments further include periodic treatments that start and stop at various times to assist with the clinical management of the patient. For example, in some embodiments, a FLAP inhibitor described herein in the combination treatment is administered weekly at the onset of treatment, decreasing to biweekly, and decreasing further as appropriate.

In certain embodiments, compositions and methods for combination therapy are provided herein. In accordance with one aspect, the pharmaceutical compositions disclosed herein are used to treat leukotriene-dependent or leukotriene mediated conditions. In accordance with another aspect, the pharmaceutical compositions disclosed herein are used to treat respiratory diseases, where treatment with a FLAP inhibitor is indicated, such as asthma, and to induce bronchodilation in a subject. In one embodiment, pharmaceutical compositions disclosed herein are used to treat a subject suffering from a vascular inflammation-driven disorder. In one embodiment, the pharmaceutical compositions disclosed herein are used to treat a subject susceptible to myocardial infarction (MI).

In certain embodiments, combination therapies described herein are used as part of a specific treatment regimen intended to provide a beneficial effect from the co-action of a FLAP inhibitors described herein and a concurrent treatment. It is understood that in certain embodiments, the dosage regimen to treat, prevent, or ameliorate the condition(s) for which relief is sought, is modified in accordance with a variety of factors. These factors include the type of respiratory disorder and the type of bronchodilation from which the subject suffers, as well as the age, weight, sex, diet, and medical condition of the subject. Thus, in some embodiments, the dosage regimen actually employed varies and, therefore, in certain embodiments, deviates from the dosage regimens set forth herein.

In certain embodiments, in combination therapies described herein, dosages of the co-administered compounds varies depending on the type of co-drug employed, on the specific drug employed, on the disease or condition being treated and so forth. In addition, in some embodiments, when co-administered with one or more biologically active agents, the compound provided herein, such as a compound of Formula (A), (B) or (C), are administered either simultaneously with the biologically active agent(s), or sequentially. In certain embodiments, when and if administered sequentially, an attending physician will decide on the appropriate sequence of administering the compound of Formula (A), (B) or (C) in combination with the second biologically active agent(s).

In certain embodiments, multiple therapeutic agents (one of which is one of the compounds described herein) are administered in any order or even simultaneously. In some embodiments simultaneous administration includes administration of a single, unified form, or of multiple forms (by way of example only, either as a single pill or as two separate pills). In certain embodiments, one of the therapeutic agents is given in multiple doses, or both are given as multiple doses. In some embodiments, wherein administration is not simultaneous, the timing between the administration of the multiple doses varies, e.g., from more than zero weeks to less than four weeks. In certain embodiments, the combination methods, compositions and formulations described herein are not to be limited to the use of only two agents; the use of multiple therapeutic combinations is also envisioned.

In certain embodiments, the compounds of Formula (A), (B) or (C) are used in combination with procedures that provide additional or synergistic benefit to a patient. By way of example only, patients are expected to find therapeutic and/or prophylactic benefit in the methods described herein, wherein pharmaceutical composition of Formula (A), (B) or (C), and/or combinations with other therapeutics are combined with genetic testing to determine whether that individual is a carrier of a mutant gene that is known to be correlated with certain diseases or conditions.

In certain embodiments, the compounds of Formula (A), (B) or (C), and combination therapies, is administered before, during or after the occurrence of a disease or condition, and the timing of administering the composition containing a compound varies. Thus, in some embodiments, the compounds are used as a prophylactic and are administered continuously to subjects with a propensity to develop conditions or diseases in order to prevent the occurrence of the disease or condition. In certain embodiments, the compounds and compositions are administered to a subject during or as soon as possible after the onset of the symptoms. In some embodiments, the administration of the compounds is initiated within the first 48 hours of the onset of the symptoms. In more specific embodiments, the administration of the compound is initiated within the first 6 hours of the onset of the symptoms; or within 3 hours of the onset of the symptoms. In certain embodiments, the initial administration is via any route practical, such as, for example, an intravenous injection, a bolus injection, infusion over 5 minutes to about 5 hours, a pill, a capsule, transdermal patch, buccal delivery, and the like, or combination thereof. In some embodiments, a compound described herein is administered as soon as is practicable after the onset of a disease or condition is detected or suspected, and for a length of time necessary for the treatment of the disease, such as, for example, from about 1 month to about 3 months. In certain embodiments, the length of treatment varies for each subject. For example, in some embodiments, a compound described herein or a formulation containing a compound described herein is administered for at least 2 weeks, for about 1 month to about 5 years, or for about 1 month to about 3 years.

In certain embodiments, therapies which combine compounds of Formula (A), (B) or (C), with inhibitors of leukotriene synthesis or leukotriene receptor antagonists, either acting at the same or other points in the leukotriene synthesis pathway, are used for treating leukotriene-dependent or leukotriene mediated diseases or conditions. In some embodiments, therapies which combine compounds of Formula (A), (B) or (C) with inhibitors of inflammation are used for treating leukotriene-dependent or leukotriene mediated diseases or conditions.

Agents to Treat Respiratory Diseases or Conditions

In some embodiments, methods for treatment of leukotriene-dependent or leukotriene mediated conditions or diseases include administering to a patient compounds, pharmaceutical compositions, or medicaments described herein in combination with other therapuetic agents that are used in the treatment of respiratory conditions or disorders, such as, but not limited to asthma. Therapuetic agents used in the treatment of respiratory conditions and disorders, such as, but not limited to asthma, include: glucocorticoids, such as, ciclesonide, beclomethasone, budesonide, flunisolide, fluticasone, mometasone, and triamcinolone; leukotriene modifiers, such as, montelukast, zafirlukast, pranlukast, and zileuton; mast cell stabilizers, such as, cromoglicate (cromolyn), and nedocromil; antimuscarinics/anticholinergics, such as, ipratropium, oxitropium, and tiotropium; methylxanthines, such as, theophylline and aminophylline; antihistamine, such as, mepyramine (pyrilamine), antazoline, diphenhydramine, carbinoxamine, doxylamine, clemastine, dimenhydrinate, pheniramine, chlorphenamine (chlorpheniramine), dexchlorphenamine, brompheniramine, triprolidine, cyclizine, chlorcyclizine, hydroxyzine, meclizine, promethazine, alimemazine (trimeprazine), cyproheptadine, azatadine, ketotifen, acrivastine, astemizole, cetirizine, loratadine, mizolastine, terfenadine, fexofenadine, levocetirizine, desloratadine, fexofenadine; omalizumab, an IgE blocker; beta2-adrenergic receptor agonists, such as: short acting beta2-adrenergic receptor agonists, such as, salbutamol (albuterol), levalbuterol, terbutaline, pirbuterol, procaterol, metaproterenol, fenoterol, bitolterol mesylate; and long-acting beta2-adrenergic receptor agonists, such as, salmeterol, formoterol, bambuterol.

Anti-Inflammatory Agents

In certain embodiments, methods for treatment of leukotriene-dependent or leukotriene mediated conditions or diseases include administering to a patient compounds, pharmaceutical compositions, or medicaments described herein in combination with an anti-inflammatory agent including, but not limited to, non-steroidal anti-inflammatory drugs (NSAIDs) and corticosteroids (glucocorticoids).

NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, COX-2 specific inhibitors (such as, but not limited to, celecoxib, rofecoxib, valdecoxib, parecoxib, etoricoxib, CS-502, JTE-522, L-745,337 and NS398).

Corticosteroids, include, but are not limited to: betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

Corticosteroids do not directly inhibit leukotriene production, therefore, in certain embodiments, co-dosing with steroids provides additional anti-inflammatory benefit.

Some commercially available anti-inflammatories include, but are not limited to: Arthrotec® (diclofenac and misoprostol), Asacol®, Salofalk® (5-aminosalicyclic acid), Auralgan® (antipyrine and benzocaine), Azulfidine® (sulfasalazine), Daypro® (oxaprozin), Lodine® (etodolac), Ponstan® (mefenamic acid), Solumedrol® (methylprednisolone), Bayer®, Bufferin® (aspirin), Indocin® (indomethacin), Vioxx® (rofecoxib), Celebrex® (celecoxib), Bextra® (valdecoxib), Arcoxia® (etoricoxib), Prexige® (lumiracoxib), Advil®, Motrin® (ibuprofen), Voltaren® (diclofenac), Orudis® (ketoprofen), Mobic® (meloxicam), Relafen® (nabumetone), Aleve®, Naprosyn® (naproxen), Feldene® (piroxicam).

In certain embodiments, the methods for treatment of respiratory diseases include administering to a patient compounds, pharmaceutical compositions, or medicaments described herein in combination with an anti-inflammatory agent.

Leukotriene Receptor Antagonists

In some embodiments, methods for treatment of leukotriene-dependent or leukotriene mediated conditions or diseases include administering to a patient compounds, pharmaceutical compositions, or medicaments described herein in combination with leukotriene receptor antagonists including, but are not limited to, CysLT₁/CysLT₂ dual receptor antagonists and CysLT₁ receptor anatagonists. In certain embodiments described herein, methods for treatment of leukotriene-dependent or leukotriene mediated conditions or diseases include administering to a patient compounds, pharmaceutical compositions, or medicaments described herein in combination with a CysLT₁/CysLT₂ dual receptor antagonist. CysLT₁/CysLT₂ dual receptor antagonists include, but are not limited to, BAY u9773, Cuthbert et al EP 00791576 (published 27 Aug. 1997), DUO-LT (Galczenski et al, D38, Poster F4 presented at American Thoracic Society, May 2002) and Tsuji et al, Org. Biomol. Chem., 1, 3139-3141, 2003. In certain embodiments, the identity of the leukotriene receptor antagonist and the amount of the leukotriene receptor antagonist and/or the amount of the compound described herein used in a method or composition described herein depends on the type of leukotriene-dependent or leukotriene mediated disorder, the time period in which the FLAP inhibitor acts to treat the disorder and the time period in which the CysLT₁/CysLT₂ dual receptor antagonist acts to inhibit CysLT receptor activity. In some embodiments, the combination treatments described herein are used for treating a patient suffering from a respiratory disorders.

In some embodiments described herein, methods for treatment of leukotriene-dependent or leukotriene mediated conditions or diseases includes administering to a patient compounds, pharmaceutical compositions, or medicaments described herein in combination with a CysLT₁ receptor antagonist. CysLT₁ receptor antagonists include, but are not limited to, zafirlukast (Accolate®), montelukast (Singulair®), prankulast (“Onon™”), and derivatives or analogs thereof. In some embodiments, such combinations are used to treat leukotriene-dependent or leukotriene mediated disorder, including respiratory disorders.

In some embodiments, the co-administration of a FLAP inhibitor described herein with a CysLT₁ receptor antagonist or a dual CysLT₁/CysLT₂ receptor antagonist has a therapeutic benefit over and above the benefit derived from the administration of a either a FLAP inhibitor or a CysLT₁R antagonist alone. In some embodiments wherein substantial inhibition of leukotriene production has undesired effects, partial inhibition of this pathway through the amelioration of the effects of the proinflammatory LTB₄ and cysteinyl leukotrienes combined with the block of the CysLT₁ receptor and/or dual CysLT₁/CysLT₂ receptor block affords substantial therapeutic benefits, particularly for respiratory diseases.

Other Combination Therapies

In some embodiments described herein, methods for treatment of leukotriene-dependent or leukotriene mediated conditions or diseases, such as proliferative disorders, including cancer, include administration to a patient compounds, pharmaceutical compositions, or medicaments described herein in combination with at least one additional agent selected from among alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, Paclitaxel™, taxol, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, dronabinol.

In certain embodiments described herein, methods for the treatment of leukotriene-dependent or leukotriene mediated conditions or diseases, such as the therapy of transplanted organs or tissues or cells, includes administration to a patient compounds, pharmaceutical compositions, or medicaments described herein in combination with at least one additional agent selected from among azathioprine, a corticosteroid, cyclophosphamide, cyclosporin, dacluzimab, mycophenolate mofetil, OKT3, rapamycin, tacrolimus, thymoglobulin.

In some embodiments described herein, methods for the treatment of leukotriene-dependent or leukotriene mediated conditions or diseases, such as atherosclerosis, include administration to a patient compounds, pharmaceutical compositions, or medicaments described herein in combination with at least one additional agent selected from among HMG-CoA reductase inhibitors (e.g., statins in their lactonized or dihydroxy open acid forms and pharmaceutically acceptable salts and esters thereof, including but not limited to lovastatin; simvastatin; dihydroxy open-acid simvastatin, particularly the ammonium or calcium salts thereof; pravastatin, particularly the sodium salt thereof; fluvastatin, particularly the sodium salt thereof; atorvastatin, particularly the calcium salt thereof; nisvastatin, also referred to as NK-104; rosuvastatin); agents that have both lipid-altering effects and other pharmaceutical activities; HMG-CoA synthase inhibitors; cholesterol absorption inhibitors such as ezetimibe; cholesterol ester transfer protein (CETP) inhibitors, for example JTT-705 and CP529, 414; squalene epoxidase inhibitors; squalene synthetase inhibitors (also known as squalene synthase inhibitors); acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; probucol; niacin; bile acid sequestrants; LDL (low density lipoprotein) receptor inducers; platelet aggregation inhibitors, such as, for example, aspirin; adenosine diphosphate (ADP) receptor inhibitors, such as, clopidogrel (Plavix®), ticlopidine (Ticlid®); phosphodiesterase inhibitors, such as, cilostazol (Pletal®); glycoprotein IIB/IIIA inhibitors, such as, abciximab (ReoPro®), eptifibatide (Integrilin®), tirofiban (Aggrastat®); adenosine reuptake inhibitors, such as, dipyridamole (Persantine®); human peroxisome proliferator activated receptor gamma (PPARγ) agonists, including the compounds commonly referred to as glitazones, for example troglitazone, pioglitazone and rosiglitazone and including those compounds included within the structural class known as thiazolidinediones as well as those PPAR-γ agonists outside the thiazolidinedione structural class; PPARα agonists such as clofibrate, fenofibrate including micronized fenofibrate, and gemfibrozil; PPAR dual α/γ agonists such as 5-[(2,4-dioxo-5-thiazolidinyl)methyl]-2-methoxy-N-[[4-(trifluoromethyl)phenyl]methyl]-benzamide, known as KRP-297; vitamin B6 (also known as pyridoxine) and the pharmaceutically acceptable salts thereof such as the HCl salt; vitamin B12 (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABC1 gene expression; FXR and LXR ligands including both inhibitors and agonists; bisphosphonate compounds such as alendronate sodium; and cyclooxygenase-2 inhibitors such as rofecoxib, celecoxib, etoricoxib, and lumiracoxib.

In certain embodiments described herein, methods for the treatment of leukotriene-dependent or leukotriene mediated conditions or diseases, such as the therapy of stroke, include administration to a patient compounds, pharmaceutical compositions, or medicaments described herein in combination with at least one additional agent selected from COX-2 inhibitors; nitric oxide synthase inhibitors, such as N-(3-(aminomethyl)benzyl)acetamidine; Rho kinase inhibitors, such as fasudil; angiotension II type-1 receptor antagonists, including candesartan, losartan, irbesartan, eprosartan, telmisartan and valsartan; glycogen synthase kinase 3 inhibitors; sodium or calcium channel blockers, including crobenetine; p38 MAP kinase inhibitors, including SKB 239063; thromboxane AX-synthetase inhibitors, including isbogrel, ozagrel, ridogrel and dazoxiben; statins (HMG CoA reductase inhibitors), including lovastatin, simvastatin, dihydroxy open-acid simvastatin, pravastatin, fluvastatin, atorvastatin, nisvastatin, and rosuvastatin; neuroprotectants, including free radical scavengers, calcium channel blockers, excitatory amino acid antagonists, growth factors, antioxidants, such as edaravone, vitamin C, TROLOX™ (6-hydroxy-2,5,7,8-tetramethylchroman-2-carboxylic acid), citicoline and minicycline, and reactive astrocyte inhibitors, such as (2R)-2-propyloctanoic acid; beta andrenergic blockers, such as propranolol, nadolol, timolol, pindolol, labetalol, metoprolol, atenolol, esmolol and acebutolol; NMDA receptor antagonists, including memantine; NR2B antagonists, such as traxoprodil; 5-HT1A agonists, such as, buspirone; adenosine diphosphate (ADP) receptor inhibitors, such as, clopidogrel (Plavix®), ticlopidine (Ticlid®); phosphodiesterase inhibitors, such as, cilostazol (Pletal®); Glycoprotein IIB/IIIA inhibitors, such as, abciximab (ReoPro®), eptifibatide (Integrilin®), tirofiban (Aggrastat®); adenosine reuptake inhibitors, such as, dipyridamole (Persantine®); receptor platelet fibrinogen receptor antagonists, including tirofiban and lamifiban; thrombin inhibitors; antithrombotics, such as argatroban; antihypertensive agents, such as: beta blockers such as, for example, atenolol, metoprolol, nadolol, oxprenolol, pindolol, propranolol, timolol; alpha blockers, doxazosin, phentolamine, indoramin, phenoxybenzamine, prazosin, terazosin, tolazoline; mixed alpha+beta blockers such as, bucindolol, carvedilol, labetalol; calcium channel blockers such as, amlodipine, felodipine, isradipine, nifedipine, nimodipine, nitrendipine, diltiazem, verapamil; ACE inhibitors such as, captopril, enalapril, fosinopril, lisinopril, perindopril, quinapril, ramipril, trandopril, benzapril; angiotensin II receptor antagonists such as, candesartan, irbesartan, losartan, telmisartan, valsartan; vasodilators, such as cyclandelate, sodium nitroprusside; nociceptin antagonists; DPIV antagonists; GABA 5 inverse agonists; and selective androgen receptor modulators.

In some embodiments described herein, methods for the treatment of leukotriene-dependent or leukotriene mediated conditions or diseases, such as the therapy of pulmonary fibrosis, include administration to a patient compounds, pharmaceutical compositions, or medicaments described herein in combination with at least one additional agent selected from anti-inflammatory agents, such as corticosteroids, azathioprine or cyclophosphamide.

In some embodiments described herein, methods for the treatment of leukotriene-dependent or leukotriene mediated conditions or diseases, such as the therapy of interstitial cystitis, include administration to a patient compounds, pharmaceutical compositions, or medicaments described herein in combination with at least one additional agent selected from dimethylsulfoxide, ornalizumab, and pentosan polysulfate.

In certain embodiments described herein, methods for the treatment of leukotriene-dependent or leukotriene mediated conditions or diseases, such as the therapy of disorders of bone, include administration to a patient compounds, pharmaceutical compositions, or medicaments described herein in combination with at least one additional agent selected from the group consisting of minerals, vitamins, bisphosphonates, anabolic steroids, parathyroid hormone or analogs, and cathepsin K inhibitors.

Treatment of Leukotriene Based Conditions or Diseases Using CysLT₁/CysLT₂ Receptor Antagonists

In accordance with certain aspects, the compositions and methods described herein are designed to deliver a CysLT₁/CysLT₂ dual receptor antagonist to block the CysLT receptor activity. The term “CysLT antagonist” or “CysLT receptor antagonist” or “leukotriene receptor antagonist” refers to a therapy that decreases the signaling of CysLTs through CysLT receptors. In some embodiments, CysLT refers to LTC₄, LTD₄ or LTE₄. Cysteinyl leukotrienes are potent smooth muscle constricting agents, particularly in respiratory and circulatory systems. In some embodiments, these are mediated via at least two cell receptors, CysLT₁ and CysLT₂. The CysLT₁ receptor and CysLT₂ receptors are G-protein-coupled receptors with seven putative transmembrane regions and an intracellular domain that interacts with G-proteins. Examples of CysLT₁/CysLT₂ dual receptor antagonists are BAY u9773, Cuthbert et al EP 00791576 (published 27 Aug. 1997), DUO-LT (Galczenski et al, D38, Poster F4 presented at American Thoracic Society, May 2002) and Tsuji et al, Org. Biomol. Chem., 1, 3139-3141, 2003.

In certain embodiments, methods for treatment of leukotriene-dependent or leukotriene mediated diseases or conditions include administering to patients compounds, pharmaceutical compositions, or medicaments that include a CysLT₁/CysLT₂ receptor antagonist. In some embodiments, such compounds, pharmaceutical compositions, or medicaments are used as treatment and/or prevention for respiratory diseases including, but not limited to, chronic stable asthma.

Diagnostic Methods for Patient Identification

In some embodiments, the screening of “leukotriene-responsive patients” which are selected for treatment with compounds of Formula (A), (B) or (C), or pharmaceutical compositions or medicaments described herein which include at least one compound of Formula (A), (B) or (C), or other FLAP modulators, are accomplished using any technique and method. In some embodiments, such techniques and methods include, by way of example, evaluation of gene haplotypes (genotype analysis), monitoring/measurement of biomarkers (phenotype analysis), monitoring/measurement of functional markers (phenotype analysis), which indicate patient response to modulators of the leukotriene pathway, or any combination thereof.

Genotype Analysis: FLAP Polymorphisms

Human FLAP has been purified and cloned and is an 18 kilodalton membrane-bound protein which is most highly expressed in human neutrophils. The FLAP gene is located at 13q12 and the gene has been linked to increased risk for both myocardial infarction and stroke in several populations. A number of polymorphisms and haplotypes in the gene encoding FLAP have been identified in individuals (U.S. Patent Application 2005113408; Sayers, Clin. Exp. Allergy, 33(8):1103-10, 2003; Kedda, et al., Clin. Exp. Allergy, 35(3):332-8, 2005). Particular FLAP haplotypes have been linked to myocardial infarction and stroke in several populations (Helgadottir A et al. Nature Genet. 36:233-239 (2004); Helgadottir A et al. Am J Hum Genet. 76:505-509 (2004); Lohmussaar E et al. Stroke 36: 731-736 (2005); Kajimoto K et al. Circ J 69:1029-1034 (2005). Previously, polymorphisms in certain genes have been demonstrated to correlate with responsiveness to given therapies, for example, the responsiveness of cancers to particular chemotherapeutic agents (Erichsen, et al., Br. J. Cancer, 90(4):747-51, 2004; Sullivan, et al., Oncogene, 23(19):3328-37, 2004). Therefore, in some embodiments, patients who are under consideration for treatment with the FLAP inhibitors described herein, or drug combinations that include such FLAP inhibitors, are screened for potential responsiveness to treatment based on their FLAP polymorphisms, or haplotypes.

Additionally, in some embodiments, polymorphisms in any of the synthetic or signaling genes dedicated to the leukotriene pathway results in a patient who is more responsive or less responsive to leukotriene modulator therapy (either FLAP or 5-LO inhibitor or leukotriene receptor antagonists). The genes dedicated to the leukotriene pathway are, e.g., 5-lipoxygenase, 5-lipoxygenase-activating protein, LTA₄ hydrolase, LTC₄ synthase, LTB₄ receptor 1 (BLT₁), LTB₄ receptor 2 (BLT₂), cysteinyl leukotriene receptor 1 (CysLT₁R), cysteinyl leukotriene receptor 2 (CysLT₂R). For example, the 5-LO gene is linked to aspirin intolerant asthma and airway hyperresponsiveness. Genetic variants in the promoter region of 5-LO predict clinical responses to a 5-LO inhibitor in asthmatics. The LTC₄ synthase gene is linked to atopy and asthma. The CysLT₂ receptor is linked to asthma and atopy. Thus, in some embodiments, polymorphisms in a leukotriene pathway gene or combination of polymorphisms or haplotypes results in altered sensitivity of the patient to therapy aimed at reducing the pathological effects of leukotrienes. In some embodiments, selection of patients who best respond to the leukotriene modulator therapies described herein include knowledge of polymorphisms in the leukotriene pathway genes and also knowledge of the expression of leukotriene-driven mediators. In some embodiments, patient selection is made on the basis of leukotriene pathway genotype alone, phenotype alone (biomarkers or functional markers) or any combination of genotype and phenotype. Choi J H et al. Hum Genet. 114:337-344 (2004); Kim, S H et al. Allergy 60:760-765 (2005); Drazen et al, Nature Genetics, 22, p 168-170, (1999); Moissidis I et al. Genet Med 7:406-410 (2005); Thompson M D et al. Pharmacogenetics 13:641-649 (2003); Pillai S G et al. Pharmacogenetics 14:627-633 (2004); Park J S et al. Pharmacogenet Genomics 15:483-492 (2005); and Fukai H et al. Pharmacogenetics 14:683-690 (2004) are hereby incorporated by reference for such disclosure.

A “haplotype,” as described herein, refers to a combination of genetic markers (“alleles”). In some embodiments, a haplotype includes one or more alleles (e.g., a haplotype containing a single SNP), two or more alleles, three or more alleles, four or more alleles, or five or more alleles. The genetic markers are particular “alleles” at “polymorphic sites” associated with FLAP. A nucleotide position at which more than one sequence is possible in a population is referred to herein as a “polymorphic site.” Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence. Alleles that differ from the reference are referred to as “variant” alleles. The term “variant FLAP” as used herein, refers to a sequence that differs from a reference FLAP sequence, but is otherwise substantially similar. The genetic markers that make up the haplotypes described herein are FLAP variants. In certain embodiments the FLAP variants are at least about 90% similar to a reference sequence. In some embodiments the FLAP variants are at least about 91% similar to a reference sequence. In some embodiments the FLAP variants are at least about 92% similar to a reference sequence. In certain embodiments the FLAP variants are at least about 93% similar to a reference sequence. In certain embodiments the FLAP variants are at least about 94% similar to a reference sequence. In certain embodiments the FLAP variants are at least about 95% similar to a reference sequence. In certain embodiments the FLAP variants are at least about 96% similar to a reference sequence. In other embodiments the FLAP variants are at least about 97% similar to a reference sequence. In certain embodiments the FLAP variants are at least about 98% similar to a reference sequence. In certain embodiments the FLAP variants are at least about 99% similar to a reference sequence.

Additionally, in certain embodiments the FLAP variants differ from the reference sequence by at least one base, while in other embodiments the FLAP variants differ from the reference sequence by at least two bases. In some embodiments the FLAP variants differ from the reference sequence by at least three bases, and in some embodiments the FLAP variants differ from the reference sequence by at least four bases.

In some embodiments, additional variants include changes that affect a polypeptide, e.g., the FLAP polypeptide. The polypeptide encoded by a reference nucleotide sequence is the “reference” polypeptide with a particular reference amino acid sequence, and polypeptides encoded by variant alleles are referred to as “variant” polypeptides with variant amino acid sequences. The FLAP nucleic acid sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence; duplication of all or a part of a sequence; transposition; or a rearrangement of a nucleotide sequence, as described in detail above. Such sequence changes alter the polypeptide encoded by a FLAP nucleic acid. For example, if the change in the nucleic acid sequence causes a frame shift, the frame shift can result in a change in the encoded amino acids, and/or can result in the generation of a premature stop codon, causing generation of a truncated polypeptide.

By way of example, a polymorphism associated with a susceptibility to myocardial infarction (MI), acute coronary syndrome (ACS), stroke or peripheral arterial occlusive disease (PAOD) can be a synonymous change in one or more nucleotides (i.e., a change that does not result in a change in the amino acid sequence). In some instances, polymorphism has a variety of effects including, for example, alter splice sites, decrease or increase expression levels, affect the stability or transport of mRNA, or otherwise affect the transcription or translation of the polypeptide. The haplotypes described below are found more frequently in individuals with MI, ACS, stroke or PAOD than in individuals without MI, ACS, stroke or PAOD. Therefore, in some embodiments, these haplotypes have predictive value for detecting a susceptibility to MI, ACS, stroke or PAOD in an individual.

Several variants of the FLAP gene have been reported to correlate with the incidence of myocardial infarction in patients (Hakonarson, JAMA, 293(18):2245-56, 2005), plus FLAP gene markers reportedly associated with the risk for developing asthma have been described in U.S. Pat. No. 6,531,279. Methods for identifying FLAP sequence variants are described, e.g., in U.S. Publication No. 2005/0113408, and in U.S. Pat. No. 6,531,279, incorporated herein by reference herein for such disclosure.

In certain embodiments, a haplotype associated with a susceptibility to myocardial infarction or stroke comprises markers SG13S99, SG13S25, SG13S377, SG13S106, SG13S32 and SG13S35 at the 13q12-13 locus. In some embodiments, the presence of the alleles T, G, G, G, A and G at SG13S99, SG13S25, SG13S377, SG13S106, SG13S32 and SG13S35, respectively (the B6 haplotype), is diagnostic of susceptibility to myocardial infarction or stroke. In some embodiments, a haplotype associated with a susceptibility to myocardial infarction or stroke comprises markers SG13S99, SG13S25, SG13S106, SG13S30 and SG13S42 at the 13q12-13 locus. In certain embodiments, the presence of the alleles T, G, G, G and A at SG13S99, SG13S25, SG13S106, SG13S30 and SG13S42, respectively (the B5 haplotype), is diagnostic of susceptibility to myocardial infarction or stroke. In some embodiments, a haplotype associated with a susceptibility to myocardial infarction or stroke comprises markers SG13S25, SG13S106, SG13S30 and SG13S42 at the 13q12-13 locus. In certain embodiments, the presence of the alleles G, G, G and A at SG13S25, SG13S106, SG13S30 and SG13S42, respectively (the 84 haplotype), is diagnostic of susceptibility to myocardial infarction or stroke. In some embodiments, a haplotype associated with a susceptibility to myocardial infarction or stroke comprises markers SG13S25, SG13S106, SG13S30 and SG13S32 at the 13q12-13 locus. In certain embodiments, the presence of the alleles G, G, G and A at SG13S25, SG13S106, SG13S30 and SG13S32, respectively (the Bs4 haplotype), is diagnostic of susceptibility to myocardial infarction or stroke. In some embodiments, patients who are under consideration for treatment with compounds of Formula (A), (B) or (C), or drug combinations described herein that include compounds of Formula (A), are screened for potential responsiveness to treatment with compounds of Formula (A), (B) or (C), based on such haplotypes.

In some embodiments, a haplotype associated with a susceptibility to myocardial infarction or stroke comprises markers SG13S99, SG13S25, SG13S114, SG13S89 and SG13S32 at the 13q12-13 locus. IN certain embodiments, the presence of the alleles T, G, T, G and A at SG13S99, SG13S25, SG13S114, SG13S89 and SG13S32, respectively (the A5 haplotype), is diagnostic of susceptibility to myocardial infarction or stroke. In some embodiments, a haplotype associated with a susceptibility to myocardial infarction or stroke comprises markers SG13S25, SG13S114, SG13S89 and SG13S32 at the 13q12-13 locus. In certain embodiments, the presence of the alleles G, T, G and A at SG13S25, SG13S114, SG13S89 and SG13S32, respectively (the A4 haplotype), is diagnostic of susceptibility to myocardial infarction or stroke. In some embodiments, patients who are under consideration for treatment with compounds of Formula (A), (B) or (C), or drug combinations described herein that include compounds of Formula (A), (B) or (C), are screened for potential responsiveness to treatment with compounds of Formula (A), (B) or (C), based on such haplotypes.

In certain embodiments, detecting haplotypes is accomplished by any suitable method for detecting sequences at polymorphic sites. In some embodiments, patients are selected using genotype selection of FLAP, 5-LO or other leukotriene pathway gene polymorphisms. In certain embodiments, the presence or absence of a leukotriene pathway gene polymorphism or haplotype is determined by any method, including, for example, using enzymatic amplification, restriction fragment length polymorphism analysis, nucleic acid sequencing, electrophoretic analysis of nucleic acid from the individual, or any combination thereof. In certain embodiments, determination of a SNP or haplotype identifies patients who will respond to, or gain benefit from, treatment with compounds of Formula (A), (B) or (C). By way of example, methods of diagnosing a susceptibility to myocardial infarction or stroke in an individual, comprises determining the presence or absence of certain single nucleotide polymorphisms (SNPs) or of certain haplotypes, wherein the presence of the SNP or the haplotype is diagnostic of susceptibility to myocardial infarction or stroke.

Phenotype Analysis: Biomarkers

In certain embodiments, patients who are under consideration for treatment with compounds of Formula (A), or drug combinations described herein that include compounds of Formula (A), (B) or (C), are screened for potential responsiveness to treatment based on leukotriene-driven inflammatory biomarker phenotypes.

In some embodiments, patient screening based on leukotriene-driven inflammatory biomarker phenotypes are used as an alternative to, or complimentary with, patient screening by leukotriene pathway gene haplotype detection. The term “biomarker” as used herein refers to a characteristic which can be measured and evaluated as an indicator of normal biological processes, pathological processes, or pharmacological responses to therapeutic intervention. Thus, in various embodiments, a biomarker is any substance, structure or process which can be measured in the body, or its products, and which may influence or predict the incidence of outcome or disease. In certain embodiments, biomarkers are classified into markers of exposure, effect, and susceptibility. In some embodiments, biomarkers are physiologic endpoints, by way of example blood pressure. In other embodiments, biomarkers are analytical endpoints, by way of example, blood glucose, or cholesterol concentrations. Techniques, used to monitor and/or measure biomarkers include, but are not limited to, NMR, LC-MS, LC-MS/MS, GC-MS, GC-MS/MS, HPLC-MS, HPLC-MS/MS, FT-MS, FT-MS/MS, ICP-MS, ICP-MS/MS, peptide/protein sequencing, nucleic acid sequencing, electrophoresis techniques, immuno-assays, immuno-blotting, in-situ hybridization, fluorescence in-situ hybridization, PCR, radio-immuno assays, and enzyme-immuno assays. In certain embodiments, single nucleotide polymorphisms (SNPs) are useful for the identification of biomarkers for propensity to certain diseases and also susceptibility or responsiveness to drugs such as chemotherapeutic agents and antiviral agents. In various embodiments, these techniques, or any combination thereof, are used to screen patients for leukotriene-dependent or leukotriene mediated diseases or conditions, wherein such patients may be beneficially treated with compounds of Formula (A), (B) or (C), or drug combinations described herein that include compounds of Formula (A), (B) or (C).

In certain embodiments, patients are selected for treatment with compounds of Formula (A), (B) or (C), or drug combinations described herein that include compounds of Formula (A), (B) or (C), by screening for enhanced inflammatory blood biomarkers such as, but not limited to, stimulated LTB₄, LTC₄, LTE₄, myeloperoxidase (MPO), eosinophil peroxidase (EPO), C-reactive protein (CRP), soluble intracellular adhesion molecule (sICAM), monocyte chemoattractant protein (MCP-1), monocyte inflammatory protein (MIP-1α), interleukin-6 (IL-6), the TH2 T cell activators interleukin 4 (IL-4), and 13 (IL-13) and other inflammatory cytokines. In certain embodiments, patients with inflammatory respiratory diseases, including but not limited to, asthma and COPD, or with cardiovascular diseases, are selected as those most likely to be responsive to leukotriene synthesis inhibition using compounds of Formula (A), (B) or (C), by using a panel of leukotriene driven inflammatory biomarkers.

Phenotype Analysis: Functional Markers

In certain embodiments, patients who are under consideration for treatment with compounds of Formula (A), (B) or (C), or drug combinations described herein that include compounds of Formula (A), (B) or (C), are screened for response to known modulators of the leukotriene pathway. In certain embodiments, patient screening by evaluation of functional markers as indicators of a patient's response to modulators of the leukotriene pathway areused as an alternative to, or complimentary with, patient screening by leukotriene pathway gene haplotype detection (genotype analysis) and/or monitoring/measurement of leukotriene-driven inflammatory biomarker phenotypes. In certain embodiments, functional markers include, but are not limited to, any physical characteristics associated with a leukotriene dependent condition or disease, or knowledge of current or past drug treatment regimens.

In some embodiments, the evaluation of lung volume and/or function is used as a functional marker for leukotriene-dependent or leukotriene mediated diseases or conditions, such as respiratory diseases. In some embodiments, lung function tests are used to screen patients, with such Leukotriene-dependent or leukotriene mediated diseases or conditions, for treatment using compounds of Formula (A), (B) or (C), or pharmaceutical compositions or medicaments which include compounds of Formula (A), (B) or (C). Such tests include, but are not limited to, evaluation of lung volumes and capacities, such as tidal volume, inspiratory reserve volume, expiratory reserve volume, residual volume, inspiratory capacity, functional residual capacity, vital capacity, total lung capacity, respiratory minute volume, alveolar ventilation, timed vital capacity, and ventilatory capacity. Methods of measuring lung volumes and capacities include, but are not limited to, maximum expiratory flow volume curve, forced expiratory volume in 1 second (FEV1), peak expiratory flow rate. In some embodiments, lung function tests used as functional markers for patient evaluation described herein include, but are not limited to, respiratory muscle power, maximum inspiratory pressure, maximum expiratory pressure, transdiaphragmatic pressure, distribution of ventilation, single breath nitrogen test, pulmonary nitrogen washout, and gas transfer.

In additional embodiments, the knowledge of a patient's past or current treatment regimen is used as a functional marker to assist in screening patients for treatment of leukotriene dependent conditions or diseases using compounds of Formula (A), (B) or (C), or pharmaceutical compositions or medicaments which include compounds of Formula (A), (B) or (C). By way of example only, such treatment regimens include past or current treatment using zileuton (Zyflo®), montelukast (Singulair®), pranlukast (Onon™), and/or zafirlukast (Accolate®).

In some embodiments, patients who are under consideration for treatment with compounds of Formula (A), (B) or (C), or drug combinations described herein that include compounds of Formula (A), (B) or (C), are screened for functional markers which include, but are not limited to, reduced eosinophil and/or basophil, and/or neutrophil, and/or monocyte and/or dendritic cell and/or lymphocyte recruitment, decreased mucosal secretion, decreased mucosal edema, and/or increased bronchodilation.

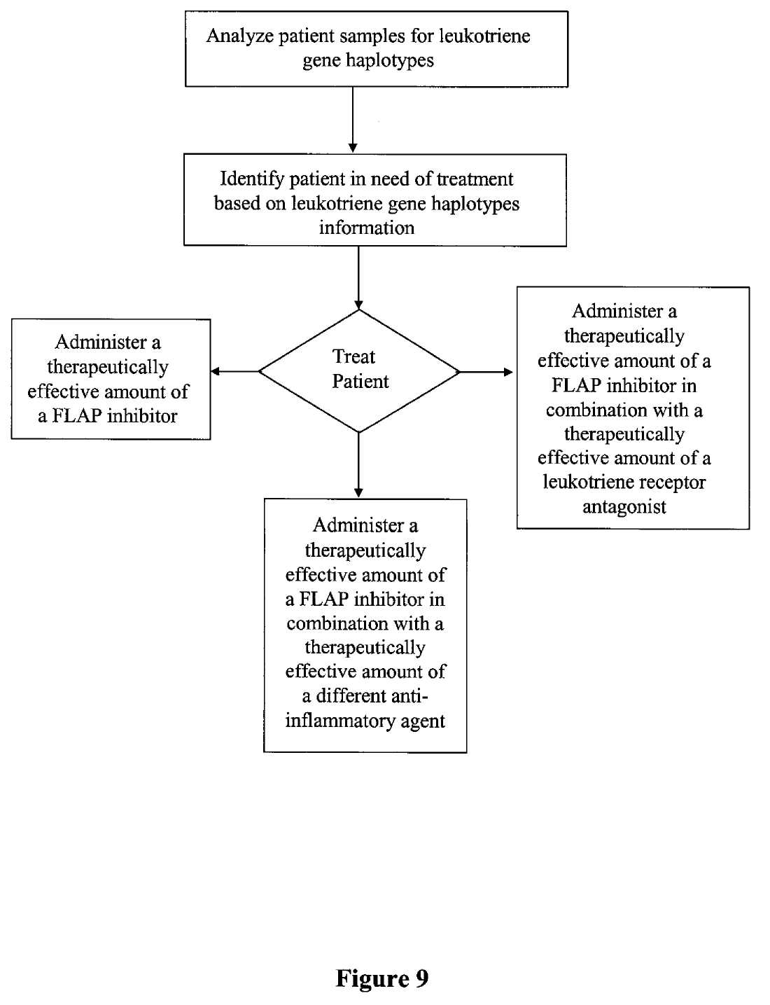

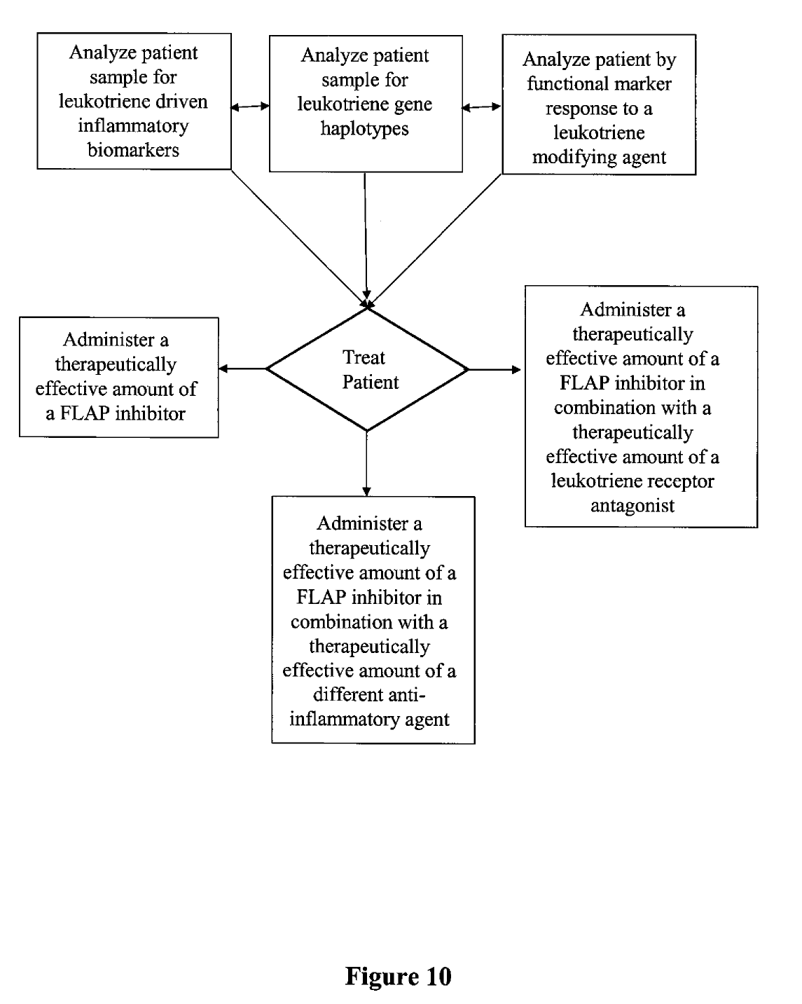

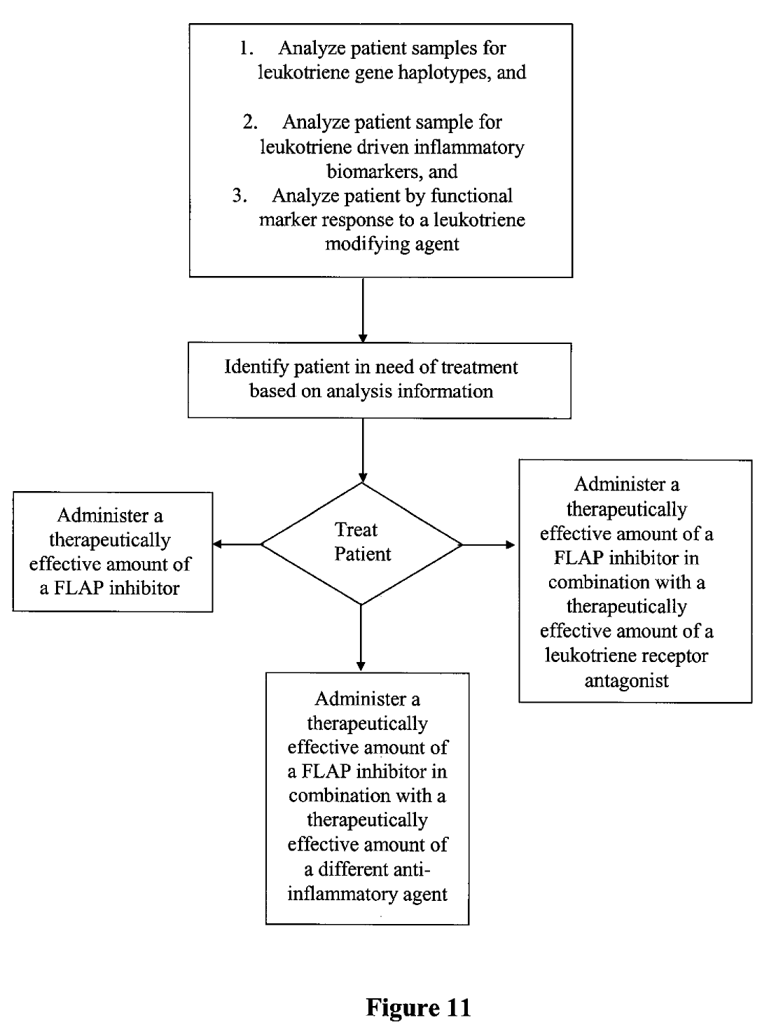

In some embodiments, methods for the identification of a patient in need of treatment for leukotriene-dependent or leukotriene mediated conditions or diseases, and exemplary, non-limiting treatment methods are shown in FIG. 9, FIG. 10 and FIG. 11, wherein a patient sample is analyzed and the information obtained is used to identify possible treatment methods. In some embodiments, in this information in conjunction with other patient information, including, but not limited to age, weight, sex, diet, and medical condition, are utilized to choose a treatment method. In some embodiments, each, any or all pieces of information will be given a particular weight in the decision process. In certain embodiments, the information obtained from the diagnostic methods described above and any other patient information, including, but not limited to age, weight, sex, diet, and medical condition, are incorporated into an algorithm used to elucidate a treatment method, wherein each piece of information will be given a particular weight in the decision process.

In certain embodiments a patient sample is analyzed for leukotriene gene haplotypes, by way of example only, FLAP haplotypes, and the information obtained identifies a patient in need of treatment using various treatment methods. Such treatment methods include, but are not limited to, administering a therapeutic effective amount of a compound of Formula (A), (B) or (C), or pharmaceutical composition or medicament which includes a compound of Formula (A), (B) or (C), administering a therapeutic effective amount of a compound of Formula (A), (B) or (C), or pharmaceutical composition or medicament which includes a compound of Formula (A), (B) or (C), in combination with a therapeutic effective amount of a leukotriene receptor antagonist (by way of example, CysLT₁/CysLT₂ antagonist or CysLT₁ antagonist), or administering a therapeutic effective amount of a compound of Formula (A), (B) or (C), or pharmaceutical composition or medicament which includes a compound of Formula (A), (B) or (C), in combination with a therapeutic effective amount of another anti-inflammatory agent. In some embodiments a patient sample is analyzed for leukotriene gene haplotypes, by way of example only, FLAP haplotypes, and/or phenotype biomarkers, and/or phenotype functional marker responses to leukotriene modifying agents. The patient may then be treated using various treatment methods. Such treatment methods include, but are not limited to, administering a therapeutic effective amount of a compound of Formula (A), (B) or (C), or pharmaceutical composition or medicament which includes a compound of Formula (A), (B) or (C), administering a therapeutic effective amount of a compound of Formula (A), (B) or (C), or pharmaceutical composition or medicament which includes a compound of Formula (A), (B) or (C) in combination with a therapeutic effective amount of a leukotriene receptor antagonist (by way of example, CysLT₁/CysLT₂ antagonist or CysLT₁ antagonist), or administering a therapeutic effective amount of a compound of Formula (A), (B) or (C), or pharmaceutical composition or medicament which includes a compound of Formula (A), (B) or (C) in combination with a therapeutic effective amount of another anti-inflammatory agent. In still other embodiments a patient sample is analyzed for leukotriene gene haplotypes, by way of example only, FLAP haplotypes, and phenotype biomarkers, and phenotype functional marker responses to leukotriene modifying agents. In some embodiments, the patient is then treated using treatment method as described herein. Such treatment methods include, but are not limited to, administering a therapeutic effective amount of a FLAP inhibitor, or pharmaceutical composition or medicament which includes a FLAP inhibitor, administering a therapeutic effective amount of a FLAP inhibitor, or pharmaceutical composition or medicament which includes a FLAP inhibitor, in combination with a therapeutic effective amount of a leukotriene receptor antagonist (by way of example, CysLT₁/CysLT₂ antagonist or CysLT, antagonist), or administering a therapeutic effective amount of a FLAP inhibitor, or pharmaceutical composition or medicament which includes a FLAP inhibitor, in combination with a therapeutic effective amount of another anti-inflammatory agent.

### Kits/Articles of Manufacture

For use in the therapeutic applications described herein, kits and articles of manufacture are also described herein. In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In some embodiments, the containers are formed from a variety of materials such as glass or plastic.

In some embodiments, the articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease, disorder, or condition that would benefit by inhibition of FLAP, or in which FLAP is a mediator or contributor to the symptoms or cause.

For example, in some embodiments, the container(s) include one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

In some embodiments, a kit includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. In some embodiments, a set of instructions is included.

In certain embodiments, a label is placed on or associated with the container. In some embodiments, a label is on a container when letters, numbers or other characters forming the label are attached, molded or etched into the container itself; and in certain embodiments, a label is associated with a container when it is present within a receptacle or carrier that also holds the container, e.g., as a package insert. In some embodiments, a label is used to indicate that the contents are to be used for a specific therapeutic application. In certain embodiments, the label indicates directions for use of the contents, such as in the methods described herein.

In certain embodiments, the pharmaceutical compositions described herein are presented in a pack or dispenser device which contains one or more unit dosage forms containing a compound provided herein. In some embodiments, the pack, for example, contains metal or plastic foil, such as a blister pack. In some embodiments, the pack or dispenser device is accompanied by instructions for administration. In certain embodiments, the pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. In some embodiments, a notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

### EXAMPLES

These examples are provided for illustrative purposes only and not to limit the scope of the claims provided herein.

### Example 1

### FLAP Binding Assays

A non-limiting example of such a FLAP binding assay is as follows: Packed human polymorphonuclear cell pellets (1.8×109 cells) (Biological Speciality Corporation) were resuspended, lysed and 100,000 g membranes prepared as described (Charleson et al. Mol. Pharmacol, 41, 873-879, 1992). 100,000×g pelleted membranes were resuspended in Tris-Tween assay buffer (100 mM Tris HCl pH 7.4, 140 mM NaCl, 2 mM EDTA, 0.5 mM DTT, 5% glycerol, 0.05% Tween 20) to yield a protein concentration of 50-100 ug/mL. 10 μL membrane suspension was added to 96 well Millipore plate, 78 μL Tris-Tween buffer, 10 μL ³H MK886 or ³H 3-[5-(pyrid-2-ylmethoxy)-3-tert-butylthio-1-benzyl-indol-2-yl]-2,2-dimethylpropionic acid (or ¹²⁵I MK591 derivative Eggler et al, J. Labelled Compounds and Radiopharmaceuticals, 1994, vXXX1V, 1147)) to ˜30,000 cpm, 2 μL inhibitor and incubated for 30 minutes at room temperature. 100 μL ice-cold washed buffer was added to the incubation mixture. Plates were then filtered and washed 3× with 200 μL ice cold Tris-Tween buffer, scintillation bottoms sealed, 100 μL scintillant added, shaken for 15 minutes then counted in a TopCount. Specific binding was determined as defined as total radioactive binding minus non-specific binding in the presence of 10 μM MK886. IC₅₀s were determined using Graphpad prism analysis of drug titration curves.

### Example 2

### Human Blood LTB₄ Inhibition Assay

A non-limiting example of such a human blood LTB₄ inhibition assay is as follows: Blood was drawn from consenting human volunteers into heparinized tubes and 125 μL aliquots added to wells containing 2.5 μL 50% DMSO (vehicle) or 2.5 μL of a test compound in 50% DMSO. Samples were incubated for 15 minutes at 37° C. 2 μL calcium ionophore A23817 (from a 50 mM DMSO stock diluted just prior to the assay in Hanks balanced salt solution (Invitrogen) to 1.25 mM) was added, solutions mixed and incubated for 30 minutes at 37° C. Samples were centrifuged at 1,000 rpm (˜200×g) for 10 minutes at 4° C., plasma removed and a 1:100 dilution assayed for LTB₄ concentration using ELISA (Assay Designs). Test compound concentrations to achieve 50% inhibition (IC₅₀'s) of vehicle LTB₄ were determined by nonlinear regression (Graphpad Prism) of % inhibition versus log test compound concentration.

### Example 3

### Rat peritoneal Inflammation and Edema Assay

A non-limiting example of such a rat peritoneal inflammation and edema assay is as follows: The in vivo efficacy of leukotriene biosynthesis inhibitors was assessed using a rat model of peritoneal inflammation. Male Sprague-Dawley rats (weighing 200-300 grams) received a single intraperitoneal (i.p.) injection of 3 mL saline containing zymosan (5 mg/mL) followed immediately by an intravenous (i.v.) injection of Evans blue dye (2 mL of 1.5% solution). Compounds were administered orally (3 mL/kg in 0.5% methylcellulose vehicle) 2 to 4 hours prior to zymosan injection. One to two hours after zymosan injection, rats were euthanized, and the peritoneal cavity was flushed with 10 mL phosphate buffered saline solution (PBS). The resulting fluid was centrifuged at 1,200 rpm for 10 minutes. Vascular edema was assesses by quantifying the amount of Evans blue dye in the supernatant using a spectrophotometer (Absorbance 610 nm). LTB₄ and cysteinyl leukotriene concentrations in the supernatant were determined by ELISA. Test compound concentrations to achieve 50% inhibition of plasma leakage (Evans blue dye) and inhibition of peritoneal LTB₄ and cysteinyl leukotrienes could be calculated by nonlinear regression (Graphpad Prism) of % inhibition versus log test compound concentration.

### Example 4

### Human Leukocyte Inhibition Assay

A non-limiting example of a human leukocyte inhibition assay is as follows: Blood was drawn from consenting human volunteers into heparanized tubes and 3% dextran, 0.9% saline equal volume added. After sedimentation of red blood cells a hypotonic lysis of remaining red blood cells was performed and leukocytes sedimented at 1000 rpm. The pellet was resuspended at 1.25×10⁵ cells/mL and aliquoted into wells containing 2.5 μL 20% DMSO (vehicle) or 2.5 μL of a test compound in 20% DMSO. Samples were incubated for 5 minutes at 37° C. and 2 μL calcium ionophore A23817 (from a 50 mM DMSO stock diluted just prior to the assay in Hanks balanced salt solution (Invitrogen)) to 1.25 mM) was added, solutions mixed and incubated for 30 minutes at 37° C. Samples were centrifuged at 1,000 rpm (˜200×g) for 10 minutes at 4° C., plasma removed and a 1:4 dilution assayed for LTB₄ concentration using ELISA (Assay Designs). Test compound concentrations to achieve 50% inhibition (IC₅₀'s) of vehicle LTB₄ were determined by nonlinear regression (Graphpad Prism) of % inhibition versus log test compound concentration.

### Example 5

### Pharmaceutical Compositions

### Example 5a

### Parenteral Composition

To prepare a parenteral pharmaceutical composition suitable for administration by injection, 100 mg of a water-soluble salt of a compound of Formula (A) is dissolved in DMSO and then mixed with 10 mL of 0.9% sterile saline. The mixture is incorporated into a dosage unit form suitable for administration by injection.

### Example 5b

### Oral Composition

To prepare a pharmaceutical composition for oral delivery, 100 mg of a compound of Formula (A) is mixed with 750 mg of starch. The mixture is incorporated into an oral dosage unit for, such as a hard gelatin capsule, which is suitable for oral administration.

### Example 5c

### Sublingual (Hard Lozenge) Composition

To prepare a pharmaceutical composition for buccal delivery, such as a hard lozenge, mix 100 mg of a compound of Formula (A) with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 5d

### Inhalation Composition

To prepare a pharmaceutical composition for inhalation delivery, 20 mg of a compound of Formula (A) is mixed with 50 mg of anhydrous citric acid and 100 mL of 0.9% sodium chloride solution. The mixture is incorporated into an inhalation delivery unit, such as a nebulizer, which is suitable for inhalation administration.

### Example 5e

### Rectal Gel Composition

To prepare a pharmaceutical composition for rectal delivery, 100 mg of a compound of Formula (A) is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 5f

### Topical Gel Composition

To prepare a pharmaceutical topical gel composition, 100 mg of a compound of Formula (A) is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topical administration.

### Example 5g

### Ophthalmic Solution Composition

To prepare a pharmaceutical ophthalmic solution composition, 100 mg of a compound of Formula (A) is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

The examples and embodiments described herein are for illustrative purposes only and various modifications or changes suggested to persons skilled in the art are to be included within the spirit and purview of this application and scope of the appended claims.

What is claimed is:

1-70. (canceled)

71. A compound having the structure of Formula (A): 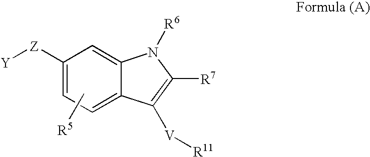
wherein:
Z is selected from among —[C(R¹)₂]ₘ—[C(R²)₂]ₙ—, —[C(R²)₂]ₙ—[C(R¹)₂]ₘ—O—, and —O—[C(R¹)₂]ₘ—[C(R²)₂]ₙ—;
each R¹ is independently H, —CF₃, or an optionally substituted C₁-C₄alkyl; or
two R¹ on the same carbon taken together with the carbon to which they are attached form a carbonyl (C═O);
each R² is independently H, —OH, —OMe, —CF₃, or an optionally substituted C₁-C₄alkyl; or
each R² can be (L^{s}R^{s}); or
two R² on the same carbon taken together with the carbon to which they are attached form a carbonyl (C═O);
m is 0, 1 or 2;
each n is independently 0, 1, 2, or 3;
Y is H, -(substituted or unsubstituted C₁-C₆alkyl), -(substituted or unsubstituted aryl), -(substituted or unsubstituted heteroaryl), or -(substituted or unsubstituted heterocycloalkyl);
where if Y or Z are substituted, then each substitutent on Y or Z is independently (L^{s}R^{s}),
each L^{s} is independently selected from among a bond, —O—, —C(═O)—, —S—, —S(═O)—, —S(═O)₂—, —NHC(═O)—, —C(═O)NH—, S(═O)₂NH—, —NHS(═O)₂, —OC(═O)NH—, —NHC(═O)O—, —OC(═O)O—, —NHC(═O)NH—, —C(═O)O—, —OC(═O)—, (substituted or unsubstituted C₁-C₆alkyl), (C₂-C₆alkenyl), (C₁-C₆fluoroalkyl), (substituted or unsubstituted heteroaryl), (substituted or unsubstituted aryl), and (substituted or unsubstituted heterocycloalkyl);
each R^{s} is independently selected from among H, halogen, —N(R⁹)₂, —CN, —NO₂, —N₃, —S(═O)₂NH₂, (substituted or unsubstituted C₁-C₆alkyl), (substituted or unsubstituted C₃-C₈cycloalkyl), (C₁-C₆fluoroalkyl), (substituted or unsubstituted aryl), (substituted or unsubstituted heteroaryl), and (substituted or unsubstituted C₁-C₆heteroalkyl);
R⁵ is H;
R⁶ is H, -L²-(substituted or unsubstituted C₁-C₆alkyl), -L²-(substituted or unsubstituted C₃-C₈cycloalkyl), -L²-(substituted or unsubstituted C₂-C₆alkenyl), -L²-(substituted or unsubstituted C₆-C₈cycloalkenyl), -L²-(substituted or unsubstituted heterocycloalkyl), -L²-(substituted or unsubstituted heteroaryl), or -L²-(substituted or unsubstituted aryl); and
L² is a bond, —S(═O)₂—, —C(═O)—, or -(substituted or unsubstituted C₁-C₆alkyl)-;
R⁷ is L³-X-L⁴-G¹, wherein,
L³ is a substituted or unsubstituted C₁-C₆alkyl;
X is a bond;
L⁴ is a bond, or a substituted or unsubstituted C₁-C₆alkyl;
G¹ is H, tetrazolyl, —OR⁹, —C(═O)NHS(═O)₂R⁸, —S(═O)₂NHC(═O)R⁸, —CN, —N(R⁹)₂, —N(R⁹)C(═O)R⁸, —NR⁹C(═CR¹⁰)N(R⁹)₂, —CO₂R⁹, —C(═O)R⁹, —CON(R⁹)₂, —SR⁸, —S(═O)R⁸, —S(═O)₂R⁸, -L⁵-(substituted or unsubstituted C₁-C₆alkyl), -L⁵-(substituted or unsubstituted heteroaryl), or -L⁵-(substituted or unsubstituted aryl);
L⁵ is —NHC(═O)O, —NHC(═O), —C(═O)NH, —C(═O)O—, or —OC(═O)—;
each R⁸ is independently selected from among (substituted or unsubstituted C₁-C₄alkyl), (substituted or unsubstituted C₃-C₈cycloalkyl), (substituted or unsubstituted phenyl), (substituted or unsubstituted heteroaryl), and (substituted or unsubstituted benzyl);
each R⁹ is independently selected from among H, (substituted or unsubstituted C₁-C₄alkyl), (substituted or unsubstituted C₃-C₈cycloalkyl), (substituted or unsubstituted phenyl), (substituted or unsubstituted heteroaryl), and (substituted or unsubstituted benzyl); or
two R⁹ groups can together form a 5-, 6-, 7-, or 8-membered heterocyclic ring; or
R⁸ and R⁹ can together form a 5-, 6-, 7-, or 8-membered heterocyclic ring; and
each R¹⁰ is independently selected from H, —S(═O)₂R⁸, —S(═O)₂NH₂, —C(O)R⁸, —CN, —NO₂, heteroaryl, or heteroalkyl;
R¹¹ is L⁷-L¹⁰-G³, wherein
L⁷ is a bond;
L¹⁰ is (substituted or unsubstituted aryl);
G³ is W⁴-G⁴, wherein
W⁴ is (substituted or unsubstituted heteroaryl); and
G⁴ is H, halogen, —CN, —NO₂, —N₃, —CF₃, —OCF₃, C₁-C₆alkyl, C₃-C₈cycloalkyl, C₁-C₆fluoroalkyl, tetrazolyl, —NHS(═O)₂R⁸, —S(═O)₂N(R⁹)₂, —OH, —OR⁸, —C(═O)CF₃, —C(O)NHS(═O)₂R⁸, —S(═O)₂NHC(O)R⁸, —CN, —N(R⁹)₂, —N(R⁹)C(O)R⁸, —C(═NR¹⁰)N(R⁹)₂, —NR⁹C(═NR¹⁰)N(R⁹)₂, —NR⁹C(═CR¹⁰)N(R⁹)₂, —C(O)NR⁹C(═NR¹⁰N(R⁹)₂, —C(O)NR⁹C(═CR¹⁰)N(R⁹)₂, —CO₂R⁹, —C(═O)R⁹, —CON(R⁹)₂, —SR⁸, —S(═O)R⁸, —S(═O)₂R⁸, -L⁹-(substituted or unsubstituted C₁-C₆alkyl), -L⁹-(substituted or unsubstituted C₂-C₆alkenyl), -L⁹-(substituted or unsubstituted C₁-C₆heteroalkyl), -L⁹-(substituted or unsubstituted heteroaryl), -L⁹-(substituted or unsubstituted heterocycloalkyl), or -L⁹-(substituted or unsubstituted aryl);
L⁹ is a bond, —O—, C(═O), —S—, —S(═O)—, —S(═O)₂—, —NH—, —NHC(═O)O—, —NHC(═O)NH—, —OC(═O)O—, —OC(═O)NH—, —NHC(═O)—, —C(═O)NH—, —C(═O)O—, or —OC(═O)—;
provided that R¹¹ comprises at least one (unsubstituted or substituted aromatic moiety) and at least one (unsubstituted or substituted heterocyclic moiety), wherein the (unsubstituted or substituted heterocyclic moiety) is a (unsubstituted or substituted heterocycloalkyl moiety) or a (unsubstituted or substituted heteroaryl moiety), and R¹¹ is not a thienyl-phenyl group;
V is a bond, —C(═O)—, —C(OH)R¹³—, —(CR¹²R¹³)—, —NH—, —C(═O)NH—, —NHC(═O)—, —S—, —S(═O)—, or —S(═O)₂—;
R¹² is H, halogen, (substituted or unsubstituted C₁-C₆alkyl), (substituted or unsubstituted C₃-C₈cycloalkyl);
R¹³ is H, (substituted or unsubstituted C₁-C₆alkyl); or
R¹² and R¹³ taken together with the carbon to which they are attached may join to form a C₃-C₈cycloalkyl; or
active metabolites, pharmaceutically acceptable solvates, pharmaceutically acceptable salts, pharmaceutically acceptable N-oxides, or pharmaceutically acceptable prodrugs thereof.

72. The compound of claim 71, wherein:
Z is selected from among —CH₂—, —CH(Me)—, —CH₂—O—, —CH(Me)—O—, —O—CH₂—, and —O—CH(Me)—.

73. The compound of claim 71, wherein:
Y is a -(substituted or unsubstituted heteroaryl), or -(substituted or unsubstituted heterocycloalkyl).

74. The compound of claim 71, wherein:
R⁶ is H, -L²-(substituted or unsubstituted C₁-C₆alkyl), -L²-(substituted or unsubstituted C₃-C₈cycloalkyl), or -L²-(substituted or unsubstituted aryl).

75. The compound of claim 71, wherein:
V is —C(═O)—, —C(OH)H—, —CR¹²H—, —S—, —S(═O)—, or —S(═O)₂—; and
G¹ is H, tetrazolyl, —OR⁹, —CO₂R⁹, —CON(R⁹)₂, -L⁵-(substituted or unsubstituted C₁-C₆alkyl), -L⁵-(substituted or unsubstituted heteroaryl).

76. A compound of claim 71, wherein the compound of Formula (A) has the structure of Formula (C):

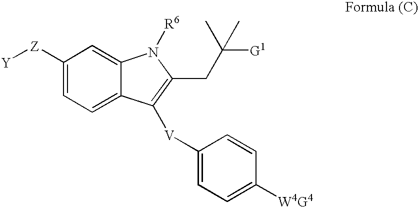

77. A compound of claim 71 having the structure of Formula (C): 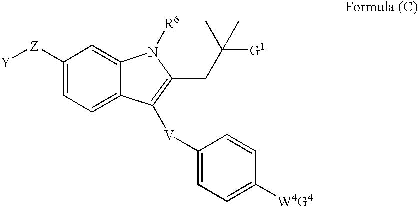
wherein:
Z is selected from —O—[C(R¹)₂]ₘ—[C(R²)₂]ₙ— and —[C(R²)₂]ₙ—[C(R¹)₂]ₘ—O—;
each R¹ is independently H, —CF₃, or an optionally substituted C₁-C₄alkyl; or
two R¹ on the same carbon taken together with the carbon to which they are attached form a carbonyl (C═O);
each R² is independently H, —OH, —OMe, —CF₃, or an optionally substituted C₁-C₄alkyl; or
two R² on the same carbon taken together with the carbon to which they are attached form a carbonyl (C═O);
m is 0, 1 or 2;
n is 0, 1, 2, or 3;
Y is -(substituted or unsubstituted aryl), -(substituted or unsubstituted heteroaryl); or (substituted or unsubstituted heterocycloalkyl);
wherein if Y is substituted, then each substitutent of Y is independently (L^{s}R^{s}),
each L^{s} is independently selected from a bond and —C(═O)—;
each R^{s} is independently selected from halogen and C₁-C₆alkyl;
R⁶ is selected from C₁-C₆alkyl, C₃-C₈cycloalkyl, C₁-C₆alkyl-C₃-C₈cycloalkyl, aryl, and C₁-C₆alkyl-aryl; and
G¹ is selected from tetrazolyl and, CO₂R⁹;
each R⁹ is independently selected from H and C₁-C₄alkyl;
W⁴ is a -(substituted or unsubstituted heteroaryl); and
G⁴ is H, halogen, C₁-C₆alkyl, C₁-C₈alkoxy, or —CF₃;
V is a bond, —(CR¹²R¹³)—, —S—, —S(═O)—, or —S(═O)₂—;
R¹² is H, halogen, or C₁-C₆alkyl;
R¹³ is H, or C₁-C₆alkyl; or
R¹² and R¹³ taken together with the carbon to which they are attached may join to form a C₃-C₈cycloalkyl; or
active metabolites, pharmaceutically acceptable solvates, pharmaceutically acceptable salts, pharmaceutically acceptable N-oxides, or pharmaceutically acceptable prodrugs thereof.

78. The compound of claim 77, wherein Z is selected from —OCH₂— and —CH₂O—; and
V is selected from —CH₂—, —S—, —S(═O)— and —S(═O)₂—.

79. A pharmaceutical composition comprising a compound, pharmaceutically acceptable salt, pharmaceutically acceptable N-oxide, pharmaceutically active metabolite, pharmaceutically acceptable prodrug, or pharmaceutically acceptable solvate of claim 71.

80. The composition of claim 79, further comprising a pharmaceutically acceptable diluent, excipient or binder.

81. A method for treating inflammation in a mammal comprising administering a therapeutically effective amount of a compound of claim 71 to the mammal in need.

82. A method for treating respiratory disease in a mammal comprising administering a therapeutically effective amount of a compound of claim 71 to the mammal in need.

83. The method of claim 82, wherein the respiratory disease is asthma.

84. A method for treating cardiovascular disease in a mammal comprising administering a therapeutically effective amount of a compound of claim 71 to the mammal in need.

85. A method of treating a leukotriene dependent or leukotriene-mediated disease or condition in a patient, comprising administering to the patient a therapeutically effective amount of the compound, pharmaceutically acceptable salt, pharmaceutically acceptable N-oxide, pharmaceutically active metabolite, pharmaceutically acceptable prodrug, or pharmaceutically acceptable solvate of claim 71.
